(19) United States
(12) Patent Application Publication
(10) Pub. No.: US 2009/0258848 A1
(43) Pub. Date: Oct. 15, 2009

(54) BIOMARKERS FOR INFLAMMATORY BOWEL DISEASE

(75) Inventors: Shukti Chakravarti, Lutherville, MD (US); Feng Wu, Lutherville, MD (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, Baltimore, MD (US)

(21) Appl. No.: 11/750,114

(22) Filed: May 17, 2007

Related U.S. Application Data
(60) Provisional application No. 60/801,663, filed on May 19, 2006.

(30) Foreign Application Priority Data
Dec. 6, 2005 (US) PCT/US05/44423

(51) Int. Cl. A61K 31/573; C07H 21/02; C12Q 1/68; A61P 1/00

(52) U.S. Cl. 514/177; 536/23.1; 435/6

(57) ABSTRACT

The present invention relates to methods of determining inflammatory bowel disease status in a subject. The invention further relates to kits for determining inflammatory bowel disease status in a subject. The invention further related to methods of identifying biomarker for determining inflammatory bowel disease status in a subject.

### RELATED APPLICATIONS

This application claims priority from U.S. Provisional Application No. 60/633,662, filed Dec. 6, 2004; PCT Application No.: PCT/US2005/44423 filed Dec. 6, 2005; and U.S. Provisional Application No. 60/801,663, filed May 19, 2006, which are incorporated herein by reference in their entirety.

### BACKGROUND OF THE INVENTION

Crohn's disease (CD) and ulcerative colitis (UC), are complex, heterogeneous, multifactorial diseases involving genetic, environmental and microbial factors. These inflammatory bowel disease (IBD) subtypes have distinctive etiopathologies yet share clinical and demographic features.¹⁻⁴ As many as 4 million people worldwide suffer from a form IBD.

Crohn's disease and ulcerative colitis have similar symptoms, but are very different in the manner in which they affect the digestive tract. Moreover, in 10% of patients with colonic disease, a distinction between UC and CD cannot be made (“indeterminate colitis”).⁵ Diagnosis and classification of these diseases are primarily based on patient histories and serologic, radiological, endoscopic and histopathology findings.⁶ Early, precise differentiation and diagnosis would directly influence the clinical treatment, patient management and the outcome of such diseases.

Thus, accurate and early diagnosis of inflammatory bowel disease is important for curative treatment interventions. Tools and methodologies for early detection and diagnosis of inflammatory bowel disease directly impacts treatment options and prognosis.

In present clinical practice, for example, screening for inflammatory bowel disease is based on clinical examination and on sigmoidoscopy or colonoscopy. Current methods for detection, diagnosis, prognosis, and treatment of IBD fails to satisfactorily reduce the morbidity associated with the disease. There is thus a need in the art for further reduction of mortality rates, and early IBD detection in minimally invasive, cost efficient formats.

### BRIEF SUMMARY OF THE INVENTION

The present invention provides, for the first time, novel biomarkers that are differentially present in the samples of inflammatory bowel disease (IBD) subjects and in the samples of control subjects. The present invention also provides sensitive and quick methods and kits that are useful for determining the inflammatory bowel disease status by measuring these novel markers. The measurement of these markers alone or in combination, in patient samples provides information that a diagnostician can correlate with a probable diagnosis of inflammatory bowel disease or a negative diagnosis (e.g., normal or disease-free). The markers are characterized by their known protein identities or by their m/z value or molecular weight and/or by characteristics discussed herein. The markers can be resolved in a sample by using a variety of techniques, e.g., microarrays, PCT techniques (e.g., real time, reverse transcriptase, PCR), and fractionation techniques (e.g., chromatographic separation coupled with mass spectrometry, protein capture using immobilized antibodies or by traditional immunoassays).

The present invention provides a method of qualifying inflammatory bowel disease status in a subject comprising measuring at least one biomarker in a sample from the subject.

In one embodiment, the method of resolution involves Surface-Enhanced Laser Desorption/Ionization (“SELDI”) mass spectrometry, in which the surface of the mass spectrometry probe comprises adsorbents that bind the markers.

In one aspect, the invention provides biomarkers for inflammatory bowel disease status comprising one or more of the following Markers 1-97, 99-211, 213-264, 266-401 and combinations thereof. These Markers 1-97, 99-211, 213-264, 266-401 are set forth in Table 1-9, which follows and are sometimes referred to herein as biomarkers of Table I or similar designations.

In one aspect, the invention provides biomarkers for inflammatory bowel disease status comprising one or more of Markers 1-97, 99-211, 213-264, 266-401 and combinations thereof.

In one embodiment, Markers 1-31, 76-97, 125-136, 187-211, 213-230, 231-264, 266-306 are Markers of Crohn's disease

In one embodiment, Markers 187-230 and 266-306 are Markers of Crohn's disease and ulcerative colitis.

In one embodiment, markers 187-211 and 266-290 are upregulated.

In one embodiment, Markers 213-230 and 291-306 are down-regulated.

In one embodiment, one or more of Markers 49-75, 99-124, 137-230, 266-306, and 307-401 are markers of ulcerative colitis.

In one embodiment, one or more of Markers 49-60, 99-124 187-211, 266-290, and 307-332 are up-regulated in ulcerative colitis.

In one embodiment, Markers 61-75, 137-186 and 333-401 are down-regulated in ulcerative colitis.

In one embodiment, one or more of Markers 76-97, 187-211, 231-245, and 266-290 are up-regulated in Crohn's Disease.

In one embodiment, Markers 125-136, 213-230, 253-261, and 291-306 are down-regulated in Crohn's Disease.

In one embodiment, Markers 1-31 are markers of Crohn's Disease.

In one embodiment, Markers 32-48 are markers of IBD.

In one embodiment, the biomarker for inflammatory bowel disease status of the invention comprises Markers 1-97, 99-211, 213-264, 266-401. In one embodiment, markers 1-48 are Markers of Crohn's disease (CD). In another embodiment, markers 49-75 are markers of ulcerative colitis. In another embodiment, markers 49-60 are up-regulated in ulcerative colitis (UC). In yet another embodiment, markers 61-75 are down-regulated in ulcerative colitis.

In other embodiments, markers 1, 2, 4 and 5 are correlate with CD; markers 6 and 10 correlate with CD; markers 17, 18, and 21 correlate with CD; markers 55 and 57 correlate with UC; markers 55 and 57 are up-regulated in UC; markers 69, 74 and 75 and are down-regulated in UC.

In one aspect, markers may discriminate between IBD disease state, for example, markers 1, 6, 17, 55 and 69 discriminate between UC and CD; markers 2, 10, 18, 57, and 74 also discriminate between UC and CD; as do markers 4, 6, 21, 55, and 69; and markers 1, 6, and 17; and markers 55 and 69.

In certain embodiments, the biomarkers may be used in combination, for example, markers 1, 2, 4 and 5; markers 6 and 10; markers 17, 18, and 21; markers 55 and 57; markers 69, 74 and 75; markers 1, 6, 17, 55 and 69; markers 2, 10, 18, 57, and 74; 4, 6, 21, 55, and 69; markers 1, 6, and 17; and markers 55 and 69.

The invention provides, in one aspect, methods for qualifying IBD status in a subject comprising measuring at least one biomarker in a sample from the subject, wherein the biomarker is selected from one or more of the biomarkers of Tables 1-9, and correlating the measurement with inflammatory bowel disease status.

In one embodiment, the inflammatory bowel disease is ulcerative colitis (UC) and/or Crohn's disease (CD).

In one embodiment, the method further comprises managing subject treatment based on the status.

In a related embodiment the managing subject treatment is selected from ordering further diagnostic tests (e.g., colonoscopy and imaging techniques), administering at least one therapeutic agent, surgery, surgery followed or preceded by at least one therapeutic agent, biotherapy, and taking no further action.

In another related embodiment, the therapeutic agent is selected from one or more of an antibiotic, an antispasmotic, and/or an antidepressant. Examples of antibiotics include, for example, rifaximin. Other therapeutic agents include, for example, sulfa drugs, corticosteriods (prednisone), 5-aminosalicylates (Asacol, Pentasa, Rowasa, or 5-ASA), immunosuppressives (azathioprine, Imuran, Cyclosporine, 6-MP, Purinethol and Methotrexate), anti-TNF (Remicade), anticholinergics, dicyclomine (Bentyl), belladonna/phenobarbital (Donnatal, Antispas, bBarbidonna, donnapine, hyosophen, Spasmolin), hyoscyamine (Levsin, Anaspaz), chlordiazepoxide/clidinium (Librax), anti-diarrheals, diphenoxylate/atropine (Lomotil), alosetron hydrochloride (Lotronex), tegaserod (Zelnorm, Zelmac), rifaximin (Xifaxin), sulfasalazine (Azulfadine), mesalamine (Asacol, Pentasa, Rowasa), osalazine (Dipentum), (Colazal), corticosteroids (prednisone), balsalazide disodium (Colazal®), cyclosporine, methotrexate, infliximab (Remicade), rifaximin, and budesonide (Entocort EC).

In one embodiment, the method for qualifying inflammatory bowel disease status in a subject may further comprise measuring the at least one biomarker after subject management.

In another embodiment, the inflammatory bowel disease status is selected from one or more of the subject's risk of IBD, the presence or absence of IBD, the type of IBD disease, the stage of IBD and effectiveness of treatment.

In another embodiment, the inflammatory bowel disease status is selected from one or more of the presence or absence of alternating diarrhea and constipation, abdominal pain, bloating, spasms, nausea, bloody diarrhea, fever, dehydration, eye inflammation, joint pain, skin rashes or lesions, mouth ulcers, chronic diarrhea, weight loss, lack of appetite, nutritional deficiencies, and/or inflamed colon.

Methods, according to one embodiment, may further comprise assessing the status of the inflammatory bowel disease, for example, by barium enema, upper GI series, stool culture, blood tests (to determine a white blood cell count or if anemia is present), fecal occult blood test, sigmoidoscopy, and/or colonoscopy.

The invention provides, in another aspect, methods for differentiating between a diagnosis of UC and CD comprising detecting in a subject sample an amount of at least one biomarker wherein the biomarker is selected from one or more of the biomarkers of Tables 1-99, and correlating the amount with a diagnosis of inflammatory bowel disease or non-inflammatory bowel disease.

TABLE 1
CD Markers

| Marker | Gene | Symbol |
|---|---|---|
| Marker 1 | Adrenomedullin** | ADM |
| Marker 2 | Serine protease inhibitor, Kazal type 1 | SPINK1 |
| Marker 3 | Serine/cysteine proteinase inhibitor, | SERPINA1 |
| | clade A, 1 | |
| Marker 4 | Signal transducer and activator of | STAT1 |
| | transcription 1 | |
| Marker 5 | Signal transducer and activator of | STAT3 |
| | transcription 3** | |
| Marker 6 | Proteasome activator subunit 2** | PSME2 |
| Marker 7 | Proteasome subunit, beta type, 8** | PSMB8 |
| Marker 8 | Ubiquitin D | UBD |
| Marker 9 | Ubiquitin-conjugating enzyme E2L 6 | UBE2L6 |
| Marker 10 | Transporter 1, ATP-binding cassette, sub B | TAP1 |
| Marker 11 | Caspase 1 | CASP1 |
| Marker 12 | Caspase 10 | CASP10 |
| Marker 13 | Acetylserotonin O-methyltransferase | ASMT |
| Marker 14 | Mucin 1, transmembrane | MUC1 |
| Marker 15 | Myosin, light polypeptide 3 | MYL3 |
| Marker 16 | Chymotrypsin-like | CTRL |
| Marker 17 | Interferon induced transmembrane | IFITM1 |
| | protein 1 | |
| Marker 18 | Interferon induced transmembrane | IFITM3 |
| | protein 3 | |
| Marker 19 | Interferon stimulated gene 20 kDa | ISG20 |
| Marker 20 | Interferon-induced protein 35** | IFI35 |
| Marker 21 | Interleukin 1, beta | IL1B |
| Marker 22 | Leukocyte Ig-like receptor, subfamily B, 1 | LILRB1 |
| Marker 23 | MHC, class II, DM alpha | HLA-DMA |
| Marker 24 | SP110 nuclear body protein | SP110 |
| Marker 25 | Chemokine (C—X—C motif) ligand 1** | CXCL1 |
| Marker 26 | Chemokine (C—X—C motif) ligand 3 | CXCL3 |
| Marker 27 | Interleukin 8 | IL8 |
| Marker 28 | Regenerating islet-derived 1 beta | REG1B |
| Marker 29 | S100 calcium binding protein A8 | S100A8 |
| Marker 30 | Lipase, gastric | LIPF |
| Marker 31 | Ig lambda variable (IV)/OR22-2 | IGLVIVOR22 |
| | | -2 |

TABLE 2
IBD Markers

| Marker | Gene | Symbol |
|---|---|---|
| Marker 32 | Ig heavy constant gamma 4 (G4m marker) | IGHG4 |
| Marker 33 | Defensin, alpha 6, Paneth cell-specific | DEFA6 |
| Marker 34 | Complement component 4 binding protein, β | C4BPB |
| Marker 35 | Decay accelerating factor for complement | DAF |
| Marker 36 | Membrane-associated protein 17 | MAP17 |
| Marker 37 | Chemokine (C—X—C motif) ligand 2 | CXCL2 |
| Marker 38 | Deleted in malignant brain tumors 1** | DMBT1 |
| Marker 39 | Interferon, alpha-inducible protein | G1P3 |
| Marker 40 | Lipocalin 2 | LCN2 |
| Marker 41 | Nitric oxide synthase 2A | NOS2A |
| Marker 42 | Pancreatitis-associated protein | PAP |
| Marker 43 | Regenerating islet-derived 1 alpha | REG1A |
| Marker 44 | S100 calcium binding protein A9 | S100A9 |
| Marker 45 | Protein kinase C, eta | PRKCH |
| Marker 46 | Regulator of G-protein signalling 3 | RGS3 |
| Marker 47 | DNA-damage-inducible transcript 4 | DDIT4 |
| Marker 48 | Hypothetical protein FLJ12443 | FLJ12443 |

TABLE 3
UC Gene Expression Signature

| Marker | Gene | Symbol |
|---|---|---|
| Up- | | |
| Regulated | | |
| Marker 49 | Defensin, alpha 5, Paneth cell-specific | DEFA5 |
| Marker 50 | Ataxia telangiectasia mutated | ATM |
| Marker 51 | Chemokine (C—X—C motif) ligand 13 | CXCL13 |
| Marker 52 | B-factor, properdin | BF |
| Marker 53 | Complement component 4A | C4A |
| Marker 54 | Actin, beta | ACTB |
| Marker 55 | Nicotinamide N-methyltransferase | NNMT |
| Marker 56 | Melanoma inhibitory activity | MIA |
| Marker 57 | Sorting nexin 26 | SNX26 |
| Marker 58 | A disintegrin and metalloproteinase domain 5 | ADAM5 |
| Marker 59 | RNA binding motif protein 8A | RBM8A |
| Marker 60 | Tribbles homolog 2 (Drosophila) | TRIB2 |
| Down- | | |
| Regulated | | |
| Marker 61 | Cyclin G1 | CCNG1 |
| Marker 62 | Myeloid/lymphoid or mixed-lineage leukemia; | MLLT3 |
| | translocated to, 3 | |
| Marker 63 | Protein phosphatase 2 (formerly 2A), regulatory | PPP2R3A |
| | subunit B″, alpha | |
| Marker 64 | Pantothenate kinase 3 | PANK3 |
| Marker 65 | Dynein, axonemal, heavy polypeptide 9 | DNAH9 |
| Marker 66 | Guanine nucleotide binding protein, gamma | GNGT1 |
| | transducing activity polypeptide 1 | |
| Marker 67 | Coagulation factor II (thrombin) receptor-like 1 | F2RL1 |
| Marker 68 | Surfactant, pulmonary-associated protein D | SFTPD |
| Marker 69 | Solute carrier family 4, sodium bicarbonate | SLC4A4 |
| | cotransporter, member 4 | |
| Marker 70 | Gamma-aminobutyric acid (GABA) A receptor, | GABRG3 |
| | gamma 3 | |
| Marker 71 | Hydroxyprostaglandin dehydrogenase 15-(NAD) | HPGD |
| Marker 72 | TAF5-like RNA polymerase II, p300/CBP- | TAF5L |
| | associated factor (PCAF)-associated factor, | |
| | 65 kDa | |
| Marker 73 | Protein kinase, cAMP-dependent, catalytic, beta | PRKACB |
| Marker 74 | | DPM1 |
| Marker 75 | | SERP1 |

TABLE 4
Genes over-expressed in CD or UC affected tissues as compared
with healthy controls

| | | Symbol |
|---|---|---|
| | CD | |
| Marker 76 | Adrenomedullin | ADM |
| Marker 77 | Serum amyloid A1 | SAA1 |
| Marker 78 | Serine/cysteine proteinase inhibitor, | SERPINA1 |
| | clade A, 1 | |
| Marker 79 | Signal transducer and activator of | STAT1 |
| | transcription 1 | |
| Marker 80 | Signal transducer and activator of | STAT3 |
| | transcription 3 | |
| Marker 81 | Leukocyte Ig-like receptor, subfamily B, | LILRB1 |
| | member 1 | |
| Marker 82 | MHC, class II, DR beta 5 | HLA-DRB5 |
| Marker 83 | Transporter 1, ATP-binding cassette, | TAP1 |
| | sub-family B | |
| Marker 84 | Proteasome activator subunit 2 (PA28 beta) | PSME2 |
| Marker 85 | Proteasome subunit, beta type, 8 | PSMB8 |
| Marker 86 | Proteasome subunit, beta type, 9 | PSMB9 |
| Marker 87 | Proteasome subunit, beta type, 10 | PSMB10 |
| Marker 88 | Interferon, alpha-inducible protein (clone | G1P3 |
| | IFI-6-16) | |
| Marker 89 | Interferon induced transmembrane protein 1 | IFITM1 |
| | (9-27) | |
| Marker 90 | Interferon induced transmembrane protein 3 | IFITM3 |
| | (1-8 U) | |
| Marker 91 | Interferon stimulated gene 20 kDa | ISG20 |
| Marker 92 | Caspase 10 | CASP10 |
| Marker 93 | Mucin 4, tracheobronchial | MUC4 |
| Marker 94 | Regenerating islet-derived 1 beta | REG1B |
| Marker 95 | Mucin 1, transmembrane | MUC1 |
| Marker 96 | Serine protease inhibitor, Kazal type 4 | SPINK4 |
| Marker 97 | Lipin 1 | LPIN1 |
| | UC | |
| Marker 99 | Coronin, actin binding protein, 1A | CORO1A |
| Marker 100 | Matrix metalloproteinase 12 | MMP12 |
| Marker 101 | Platelet/endothelial cell adhesion molecule | PECAM1 |
| | (CD31) | |
| Marker 102 | Talin 1 | TLN1 |
| Marker 103 | Tissue inhibitor of metalloproteinase 1 | TIMP1 |
| Marker 104 | Interferon, gamma-inducible protein 30 | IFI30 |
| Marker 105 | POU domain, class 2, associating factor 1 | POU2AF1 |
| Marker 106 | Clusterin (complement lysis inhibitor, | CLU |
| | SP-40, 40) | |
| Marker 107 | TNF receptor superfamily, member 7 | TNFRSF7 |
| Marker 108 | Prostaglandin D2 synthase | PTGDS |
| Marker 109 | CD79A antigen (Ig-associated alpha) | CD79A |
| Marker 110 | Defensin, alpha 5, Paneth cell-specific | DEFA5 |
| Marker 111 | Ubiquitin D | UBD |
| Marker 112 | Chemokine (C-C motif) ligand 11 | CCL11 |
| Marker 113 | Insulin-like growth factor binding protein 5 | IGFBP5 |
| Marker 114 | Endothelial cell growth factor 1 (platelet- | ECGF1 |
| | derived) | |
| Marker 115 | Fascin homolog 1, actin-bundling protein | FSCN1 |
| Marker 116 | Ataxia telangiectasia mutated | ATM |
| Marker 117 | Notch homolog 3 (Drosophila) | NOTCH3 |
| Marker 118 | Protease inhibitor 3, skin-derived (SKALP) | PI3 |
| Marker 119 | Nucleoporin 210 | NIP210 |
| Marker 120 | AT rich interactive domain 5A (MRF1-like) | ARID5A |
| Marker 121 | Pyruvate dehydrogenase kinase, isoenzyme 3 | PDK3 |
| Marker 122 | Cathepsin H | CTSH |
| Marker 123 | Lymphocyte cytosolic protein 1 (L-plastin) | LCP1 |
| Marker 124 | Stomatin | STOM |

TABLE 5
Genes down-regulated in CD or UC as compared with healthy controls

| | | Symbol |
|---|---|---|
| | CD | |
| Marker 125 | Down syndrome critical region gene 1-like 1 | DSCR1L1 |
| Marker 126 | Spondin 1, extracellular matrix protein | SPON1 |
| Marker 127 | Thrombospondin 1 | THBS1 |
| Marker 128 | Chemokine (C—X—C motif) ligand 12 | CXCL12 |
| Marker 129 | Stathmin-like 2 | STMN2 |
| Marker 130 | Serine/cysteine proteinase inhibitor, clade B, 7 | SERPINB7 |
| Marker 131 | WEE1 homolog (S. pombe) | WEE1 |
| Marker 132 | Myosin, heavy polypeptide 11, smooth muscle | MYH11 |
| Marker 133 | Chromosome 14 ORF116 (checkpoint suppressor 1) | CHES1 |
| Marker 134 | Pre-B-cell leukemia transcription factor 3 | PBX3 |
| Marker 135 | Autism susceptibility candidate 2 | AUTS2 |
| Marker 136 | Poliovirus receptor-related 3 | PVRL3 |
| | UC | |
| Marker 137 | Semaphorin 6A-1 | SEMA6A |
| Marker 138 | KIAA0931 protein (PH domain and leucine rich Repeat protein | PHLPPL |
| | phosphatase-like) | |
| Marker 139 | Mitochondrial ribosomal protein S6 | MRPS6 |
| Marker 140 | Sterol-C5-desaturase (ERG3 delta-5-desaturase Homolog, fungal)- | SC5DL |
| | like | |
| Marker 141 | Related RAS viral (r-ras) oncogene homolog 2 | SCP2 |
| Marker 142 | UDP-glucose dehydrogenase | UGDH |
| Marker 143 | Calpastatin | CAST |
| Marker 144 | ADAM-like, decysin 1 | ADAMDEC1 |
| Marker 145 | Dynein, axonemal, heavy polypeptide 9 | DNAH9 |
| Marker 146 | Ephrin-A1 | EENA1 |
| Marker 147 | Fibroblast growth factor receptor 3 | FGFR3 |
| Marker 148 | Methylmalonyl Coenzyme A mutase | MUT |
| Marker 149 | Phosphoenolpyruvate carboxykinase 1 (soluble) | PCK1 |
| Marker 137 | Gamma-glutamyl hydrolase | GGH |
| Marker 138 | N-acylsphingosine amidohydrolase-like | ASAHL |
| Marker 139 | Acyl-Coenzyme A dehydrogenase, C-4 to C-12 straight chain | ACADM |
| Marker 140 | UDP glycosyltransferase 2 family, B28 | UGT2B28 |
| Marker 141 | Ectonucleoside triphosphate diphosphohydrolase 5 | ENTPD5 |
| Marker 142 | Ectonucleotide pyrophosphatase/phosphodiesterase 4 | ENPP4 |
| Marker 143 | Cisplatin resistance associated | MTMR11 |
| Marker 144 | aAcyl-Coenzyme A oxidase 1, palmitoyl | ACOX1 |
| Marker 145 | Neural precursor cell expressed, developmentally down-regulated 4- | NEDD4L |
| | like | |
| Marker 146 | Tetraspanin 7 (transmembrane 4 superfamily, 2) | TSPAN7 |
| Marker 147 | Protein tyrosine phosphatase, receptor type, R | PTPRR |
| Marker 148 | Vacuolar protein sorting 13A (yeast) | VPS13A |
| Marker 149 | Procollagen-lysine, 2-oxoglutarate 5-dioxygenase 2 | PLOD2 |
| Marker 150 | Dual-specificity tyrosine-(Y)-phosphorylation regulated kinase 2 | DYRK2 |
| Marker 151 | Guanylate cyclase activator 2A (guanylin) | GUCA2A |
| Marker 152 | Guanylate cyclase activator 2B (uroguanylin) | GUCA2B |
| Marker 153 | Sorcin | SRI |
| Marker 154 | Endothelin 3 | EDN3 |
| Marker 155 | Peroxiredoxin 6 | PRDX6 |
| Marker 156 | Selenium binding protein 1 | SELENBP1 |
| Marker 157 | A kinase (PRKA) anchor protein (yotiao) 9 | AKAP9 |
| Marker 158 | Phosphoinositide-3-kinase, regulatory subunit, polypeptide 1 (p85 | PIK3R1 |
| | alpha) | |
| Marker 159 | Coagulation factor II (thrombin) receptor-like 1 | F2RL1 |
| Marker 160 | Lectin, galactoside-binding, soluble, 2 (galectin 2) | LGALS2 |
| Marker 161 | | |
| Marker 162 | Chromodomain helicase DNA binding protein 1 | CHD1 |
| Marker 163 | Hepatocyte nuclear factor 4, gamma | HNF4G |
| Marker 164 | Myeloid/lymphoid or mixed-lineage leukemia (trithorax homolog, | MLLT2 |
| | Drosophila); translocated to, 2 | |
| Marker 165 | v-myb myeloblastosis viral oncogene homolog (avian) | MYB |
| Marker 166 | Nuclear receptor subfamily 3, group C, member 2 | NR3C2 |
| Marker 167 | SATB family member 2 | SATB2 |
| Marker 168 | Zinc finger protein 217 | ZNF217 |
| Marker 169 | Cyclin T2 | CCNT2 |
| Marker 170 | Kruppel-like factor 5 (intestinal) | KLF5 |
| Marker 171 | ATPase, Ca++ transporting, plasma membrane 1 | ATP2B1 |
| Marker 172 | Exophilin 5 | EXPH5 |
| Marker 173 | Solute carrier family 16, member 1 | SLC16A1 |
| Marker 174 | Secretory carrier membrane protein 1 | SCAMP1 |
| Marker 175 | Transportin 1 | TNPO1 |
| Marker 176 | Solute carrier family 26, member 2 | SLC26A2 |
| Marker 177 | Aquaporin 8 | AQP8 |
| Marker 178 | Peptidyl arginine deiminase, type II | — |
| Marker 179 | Cordon-bleu homolog (mouse) | COBL |
| Marker 180 | Family with sequence similarity 8, member A1 | FAM8A1 |
| Marker 181 | Hypothetical protein FLJ13910 | FLJ13910 |
| Marker 182 | GRP1-binding protein GRSP1(FERM domain containing 4B) | FRMD4B |
| Marker 183 | Histone 1, H4c | HIST1H4C |
| Marker 184 | Hepatocellular carcinoma antigen gene 520 | LOC63928 |
| Marker 185 | Hypothetical protein LOC92482 | LOC92482 |
| Marker 186 | FLJ11220 (round spermatid basic protein 1) | RSBN1 |

TABLE 6
Gene expression changes in CD and UC as compared
with healthy controls

| | | Symbol |
|---|---|---|
| | Up-regulated | |
| Marker 187 | Ig heavy constant gamma 4 (G4m marker) | IGHG4 |
| Marker 188 | MHC, class II, DM alpha | HLA-DMA |
| Marker 189 | MHC, class II, DR beta 1 | HLA-DRB1 |
| Marker 190 | Defensin, alpha 6, Paneth cell-specific | DEFA6 |
| Marker 191 | Chemokine (C—X—C motif) ligand 1 | CXCL1 |
| Marker 192 | Chemokine (C—X—C motif) ligand 2 | CXCL2 |
| Marker 193 | Chemokine (C—X—C motif) ligand 3 | CXCL3 |
| Marker 194 | Interleukin 8 | IL8 |
| Marker 195 | B-factor, properdin | BF |
| Marker 196 | Decay accelerating factor for complement | DAF |
| Marker 197 | Deleted in malignant brain tumors 1 | DMBT1 |
| Marker 198 | Lipocalin 2 (oncogene 24p3) | LCN2 |
| Marker 199 | Nitric oxide synthase 2A (inducible, | NOS2A |
| | hepatocytes) | |
| Marker 200 | Regenerating islet-derived 3 alpha | REG3A |
| Marker 201 | S100 calcium binding protein A9 (MRP14) | S100A9 |
| Marker 202 | Caspase 1 | CASP1 |
| Marker 203 | Peptidylprolyl isomerase D | PPID |
| Marker 204 | Pim-2 oncogene | PIM2 |
| Marker 205 | Regenerating islet-derived 1 alpha | REG1A |
| Marker 206 | Tryptophanyl-tRNA synthetase | WARS |
| Marker 207 | Regulator of G-protein signalling 3 | RGS3 |
| Marker 208 | Hypothetical protein FLJ12443 | FLJ12443 |
| Marker 209 | Protein serine kinase H1 | PSKH1 |
| Marker 210 | Ubiquitin-conjugating enzyme E2L 6 | UBE2L6 |
| Marker 211 | PDZK1 interacting protein 1 | PDZK1IP1 |
| | Down-regulated | |
| Marker 213 | Adducin 3 (gamma) | ADD3 |
| Marker 214 | Claudin 8 | CLDN8 |
| Marker 215 | Protein kinase C, iota | PRKCI |
| Marker 216 | UDP glycosyltransferase 8 | UGT8 |
| Marker 217 | BTB (POZ) domain containing 3 | BTBD3 |
| Marker 218 | Protein kinase C-like 2 | PKN2 |
| Marker 219 | Protein kinase, cAMP-dependent, | PRKACB |
| | catalytic, beta | |
| Marker 220 | ATP-binding cassette, sub-family B | ABCB1 |
| | (MDR/TAP), 1 | |
| Marker 221 | Solute carrier family 4, member 4 | SLC4A4 |
| Marker 222 | MAX interactor 1 | MXI1 |
| Marker 223 | Sp3 transcription factor | SP3 |
| Marker 224 | Frizzled-related protein | FRZB |
| Marker 225 | Fk506-Binding Protein, Alt. Splice 2 | — |
| Marker 226 | mRNA; cDNA DKFZp586B211 | — |
| Marker 227 | Chromosome 14 open reading frame 11 | C14orf11 |
| Marker 228 | Creatine kinase, brain | CKB |
| Marker 229 | Transcribed sequences | KIAA1651 |
| Marker 230 | Putative MAPK activating protein | TIPRL |

TABLE 7
Differential gene expression in affected CD compared to
healthy control

| | | Symbol |
|---|---|---|
| | Up-regulated Gene | |
| Marker 231 | Adrenomedullin | ADM |
| Marker 232 | Serum amyloid A1 | SAA1 |
| Marker 233 | Serine/cysteine proteinase inhibitor, | SERPINA1 |
| | clade A, 1 | |
| Marker 234 | Signal transducer and activator | STAT1 |
| | of transcription 1 | |
| Marker 235 | Signal transducer and activator | STAT3 |
| | of transcription 3 | |
| Marker 236 | MHC, class II, DR beta 5 | HLA-DRB5 |
| Marker 237 | Transporter 1, ATP-binding cassette, | TAP1 |
| | sub-family B | |
| Marker 238 | Proteasome activator subunit 2 (PA28 beta) | PSME2 |
| Marker 239 | Proteasome subunit, beta type, 8 | PSMB8 |
| Marker 240 | Proteasome subunit, beta type, 9 | PSMB9 |
| Marker 241 | Proteasome subunit, beta type, 10 | PSMB10 |
| Marker 242 | Interferon, alpha-inducible protein | G1P3 |
| | (clone IFI-6-16) | |
| Marker 243 | Leukocyte Ig-like receptor, subfamily B, | LILRB1 |
| | member 1 | |
| Marker 244 | Interferon induced transmembrane | IFITM1 |
| | protein 1 (9-27) | |
| Marker 245 | Interferon induced transmembrane | IFITM3 |
| | protein 3 (1-8U) | |
| Marker 246 | Interferon stimulated gene 20 kDa | ISG20 |
| Marker 247 | Caspase 10 | CASP10 |
| Marker 248 | Mucin 4, tracheobronchial | MUC4 |
| Marker 249 | Regenerating islet-derived 1 beta | REG1B |
| Marker 250 | Mucin 1, transmembrane | MUC1 |
| Marker 251 | Serine protease inhibitor, Kazal type 4 | SPINK4 |
| Marker 252 | Lipin 1 | LPIN1 |
| | Down-regulated | |
| Marker 253 | Down syndrome critical region gene 1-like 1 | DSCR1L1 |
| Marker 254 | Spondin 1, extracellular matrix protein | SPON1 |
| Marker 255 | Thrombospondin 1 | THBS1 |
| Marker 256 | Chemokine (C—X—C motif) ligand 12 | CXCL12 |
| Marker 257 | Stathmin-like 2 | STMN2 |
| Marker 258 | Serine/cysteine proteinase inhibitor, | SERPINB7 |
| | clade B, 7 | |
| Marker 259 | WEE1 homolog (S. pombe) | WEE1 |
| Marker 260 | Myosin, heavy polypeptide 11, | MYH11 |
| | smooth muscle | |
| Marker 261 | Chromosome 14 ORF116 | CHES1 |
| | (checkpoint suppressor 1) | |
| Marker 262 | Pre-B-cell leukemia transcription factor 3 | PBX3 |
| Marker 263 | Autism susceptibility candidate 2 | AUTS2 |
| Marker 264 | Poliovirus receptor-related 3 | PVRL3 |
| Marker 265 | | |

TABLE 8
Gene expression overlaps in CD and UC compared to healthy control

| | | Symbol |
|---|---|---|
| | Up-regulated | |
| Marker 266 | Ig heavy constant gamma 4 (G4m marker) | IGHG4 |
| Marker 267 | MHC, class II, DM alpha | HLA-DMA |
| Marker 268 | MHC, class II, DR beta 1 | HLA-DRB1 |
| Marker 269 | Defensin, alpha 6, Paneth cell-specific | DEFA6 |
| Marker 270 | Chemokine (C—X—C motif) ligand 1 | CXCL1 |
| Marker 271 | Chemokine (C—X—C motif) ligand 2 | CXCL2 |
| Marker 272 | Chemokine (C—X—C motif) ligand 3 | CXCL3 |
| Marker 273 | Interleukin 8 | IL8 |
| Marker 274 | B-factor, properdin | BF |
| Marker 275 | Decay accelerating factor for complement | DAF |
| Marker 276 | Deleted in malignant brain tumors 1 | DMBT1 |
| Marker 277 | Lipocalin 2 (oncogene 24p3) | LCN2 |
| Marker 278 | Nitric oxide synthase 2A (inducible, hepatocytes) | NOS2A |
| Marker 279 | Regenerating islet-derived 3 alpha | REG3A |
| Marker 280 | S100 calcium binding protein A9 (MRP14) | S100A9 |
| Marker 281 | Caspase 1 | CASP1 |
| Marker 282 | Peptidylprolyl isomerase D (Cyclophilin D) | PPID |
| Marker 283 | Pim-2 oncogene | PIM2 |
| Marker 284 | Regenerating islet-derived 1 alpha | REG1A |
| Marker 285 | Tryptophanyl-tRNA synthetase | WARS |
| Marker 286 | Regulator of G-protein signalling 3 | RGS3 |
| Marker 287 | Hypothetical protein FLJ12443 | FLJ12443 |
| Marker 288 | Protein serine kinase H1 | PSKH1 |
| Marker 289 | Ubiquitin-conjugating enzyme E2L 6 | UBE2L6 |
| Marker 290 | PDZK1 interacting protein 1 For Peer Review | PDZK1IP1 |
| | Down-regulated | |
| Marker 291 | Adducin 3 (gamma) | ADD3 |
| Marker 292 | Claudin 8 Protein kinase C, iota | CLDN8 PRKCI |
| Marker 293 | UDP glycosyltransferase 8 | UGT8 |
| Marker 294 | BIB (POZ) domain containing 3 | BTBD3 |
| Marker 295 | Protein kinase C-like 2 | PKN2 |
| Marker 296 | Protein kinase, cAMP-dependent, catalytic, beta | PRKACB |
| Marker 297 | ATP-binding cassette, sub-family B (MDR/TAP), 1 Solute | ABCB1 SLC4A4 |
| | carrier family 4, member 4 | |
| Marker 298 | MAX interactor 1 | MXI1 |
| Marker 299 | Sp3 transcription factor | SP3 |
| Marker 300 | Frizzled-related protein | FRZB |
| Marker 301 | Fk506-Binding Protein, Alt. Splice 2 | — |
| Marker 302 | mRNA; cDNA DKFZp586B211 | — |
| Marker 303 | Chromosome 14 open reading frame 11 | C14orf11 |
| Marker 304 | Creatine kinase, brain | CKB |
| Marker 305 | Transcribed sequences | KIAA1651 |
| Marker 306 | Putative MAPK activating protein | TIPRL |

TABLE 9
Differential gene expression in affected UC tissues compared to healthy control

| | | Symbol |
|---|---|---|
| | Up-regulated Gene | |
| Marker 307 | Coronin, actin binding protein, 1A | CORO1A |
| Marker 308 | Matrix metalloproteinase 12 | MMP12 |
| Marker 309 | Platelet/endothelial cell adhesion molecule (CD31) | PECAM1 |
| Marker 310 | Talin 1 | TLN1 |
| Marker 311 | Tissue inhibitor of metalloproteinase 1 | TIMP1 |
| Marker 312 | Interferon, gamma-inducible protein 30 | IFI30 |
| Marker 313 | POU domain, class 2, associating factor 1 | POU2AF1 |
| Marker 314 | Clusterin (complement lysis inhibitor, SP-40,40) | CLU |
| Marker 315 | TNF receptor superfamily, member 7 | TNFRSF7 |
| Marker 316 | Prostaglandin D2 synthase | PTGDS |
| Marker 317 | CD79A antigen (Ig-associated alpha) For Peer Review | CD79A |
| Marker 318 | Defensin, alpha 5, Paneth cell-specific | DEFA5 |
| Marker 319 | Ubiquitin D | UBD |
| Marker 320 | Chemokine (C-C motif) ligand 11 | CCL11 |
| Marker 321 | Insulin-like growth factor binding protein 5 | IGFBP5 |
| Marker 322 | Endothelial cell growth factor 1 (platelet-derived) | ECGF1 |
| Marker 323 | Fascin homolog 1, actin-bundling protein | FSCN1 |
| Marker 324 | Ataxia telangiectasia mutated | ATM |
| Marker 325 | Notch homolog 3 (Drosophila) | NOTCH3 |
| Marker 326 | Protease inhibitor 3, skin-derived (SKALP) | PI3 |
| Marker 327 | Nucleoporin 210 | NIP210 |
| Marker 328 | AT rich interactive domain 5A (MRF1-like) | ARID5A |
| Marker 329 | Pyruvate dehydrogenase kinase, isoenzyme 3 | PDK3 |
| Marker 330 | Cathepsin H | CTSH |
| Marker 331 | Lymphocyte cytosolic protein 1 (L-plastin) | LCP1 |
| Marker 332 | Stomatin | STOM |
| | Down-regulated | |
| Marker 333 | Semaphorin 6A-1 | SEMA6A |
| Marker 334 | KIAA0931 protein (PH domain and leucine rich | PHLPPL |
| Marker 335 | Repeat protein phosphatase-like) | |
| Marker 336 | Mitochondrial ribosomal protein S6 | MRPS6 |
| Marker 337 | Sterol-C5-desaturase (ERG3 delta-5-desaturase | SC5DL |
| Marker 338 | Homolog, fungal)-like | |
| Marker 339 | Related RAS viral (r-ras) oncogene homolog 2 | SCP2 |
| Marker 340 | UDP-glucose dehydrogenase | UGDH |
| Marker 341 | Calpastatin | CAST |
| Marker 342 | ADAM-like, decysin 1 | ADAMDEC1 |
| Marker 343 | Dynein, axonemal, heavy polypeptide 9 | DNAH9 |
| Marker 344 | Ephrin-A1 | EFNA1 |
| Marker 345 | Fibroblast growth factor receptor 3 | FGFR3 |
| Marker 346 | Methylmalonyl Coenzyme A mutase | MUT |
| Marker 347 | Phosphoenolpyruvate carboxykinase 1 (soluble) | PCK1 |
| Marker 348 | Gamma-glutamyl hydrolase | GGH |
| Marker 349 | N-acylsphingosine amidohydrolase-like | ASAHL |
| Marker 350 | Acyl-Coenzyme A dehydrogenase, | ACADM |
| Marker 351 | UDP glycosyltransferase 2 family, B28 | UGT2B28 |
| Marker 352 | Ectonucleoside triphosphate diphosphohydrolase 5 | ENTPD5 |
| Marker 353 | Ectonucleotide pyrophosphatase/phosphodiesterase 4 | ENPP4 |
| Marker 354 | Cisplatin resistance associated | MTMR11 |
| Marker 355 | aAcyl-Coenzyme A oxidase 1, palmitoyl | ACOX1 |
| Marker 356 | Neural precursor cell expressed, developmentally | NEDD4L |
| Marker 357 | down-regulated 4-like | |
| Marker 358 | Tetraspanin 7 (transmembrane 4 superfamily, 2) | TSPAN7 |
| Marker 359 | Protein tyrosine phosphatase, receptor type, R | PTPRR |
| Marker 360 | Vacuolar protein sorting 13A (yeast) | VPS13A |
| Marker 361 | Procollagen-lysine, 2-oxoglutarate 5-dioxygenase 2 | PLOD2 |
| Marker 362 | Dual-specificity tyrosine-(Y)-phosphorylation | DYRK2 |
| Marker 363 | regulated kinase 2 | |
| Marker 364 | Guanylate cyclase activator 2A (guanylin) | GUCA2A |
| Marker 365 | Guanylate cyclase activator 2B (uroguanylin) | GUCA2B |
| Marker 366 | Sorcin | SRI |
| Marker 367 | Endothelin 3 | EDN3 |
| Marker 368 | Peroxiredoxin 6 | PRDX6 |
| Marker 369 | Selenium binding protein 1 | SELENBP1 |
| Marker 370 | A kinase (PRKA) anchor protein (yotiao) 9 | AKAP9 |
| Marker 371 | Phosphoinositide-3-kinase, regulatory subunit, | PIK3R1 |
| Marker 372 | polypeptide 1 (p85 alpha) For Peer Review | |
| Marker 373 | Coagulation factor II (thrombin) receptor-like 1 | F2RL1 |
| Marker 374 | Lectin, galactoside-binding, soluble, 2 (galectin 2) | LGALS2 |
| Marker 375 | Chromodomain helicase DNA binding protein 1 | CHD1 |
| Marker 376 | Hepatocyte nuclear factor 4, gamma | HNF4G |
| Marker 377 | Myeloid/lymphoid or mixed-lineage leukemia | MLLT2 |
| Marker 378 | (trithorax homolog, Drosophila); translocated to, 2 | |
| Marker 379 | v-myb myeloblastosis viral oncogene homolog (avian) | MYB |
| Marker 380 | Nuclear receptor subfamily 3, group C, member 2 | NR3C2 |
| Marker 381 | SATB family member 2 | SATB2 |
| Marker 382 | Zinc finger protein 217 | ZNF217 |
| Marker 383 | Cyclin T2 | CCNT2 |
| Marker 384 | Kruppel-like factor 5 (intestinal) | KLF5 |
| Marker 385 | ATPase, Ca++ transporting, plasma membrane 1 | ATP2B1 |
| Marker 386 | Exophilin 5 | EXPH5 |
| Marker 387 | Solute carrier family 16, member 1 | SLC16A1 |
| Marker 388 | Secretory carrier membrane protein 1 | SCAMP1 |
| Marker 389 | Transportin 1 | TNPO1 |
| Marker 390 | Solute carrier family 26, member 2 | SLC26A2 |
| Marker 391 | Aquaporin 8 | AQP8 |
| Marker 392 | Peptidyl arginine deiminase, type II | — |
| Marker 393 | Cordon-bleu homolog (mouse) | COBL |
| Marker 394 | Family with sequence similarity 8, member A1 | FAM8A1 |
| Marker 395 | Hypothetical protein FLJ13910 | FLJ13910 |
| Marker 396 | GRP1-binding protein GRSP1(FERM domain | FRMD4B |
| Marker 397 | containing 4B) | |
| Marker 398 | Histone 1, H4c | HIST1H4C |
| Marker 399 | Hepatocellular carcinoma antigen gene 520 | LOC63928 |
| Marker 400 | Hypothetical protein LOC92482 | LOC92482 |
| Marker 401 | FLJ11220 (round spermatid basic protein 1) | RSBN1 |

Markers of the invention may be detected, for example, by mass spectrometry according to one embodiment. In a related embodiment, the markers are detected by SELDI. In another related embodiment, the marker or markers are detected by capturing the marker on a biochip having a hydrophobic surface and detecting the captured marker by SELDI. Suitable biochips include the IMAC3 ProteinChip® Array and the WCX2 ProteinChip® Array. In another related embodiment, markers are detected by nucleic acid arrays, e.g., DNA arrays or by PCR methods.

In one embodiment, the methods for qualifying inflammatory bowel disease status in a subject further comprise generating data on immobilized subject samples on a biochip, by subjecting the biochip to laser ionization and detecting intensity of signal for mass/charge ratio; and transforming the data into computer readable form; executing an algorithm that classifies the data according to user input parameters, for detecting signals that represent biomarkers present in inflammatory bowel disease subjects and are lacking in non-inflammatory bowel disease subject controls.

In one embodiment, one or more of the biomarkers are detected using laser desorption/ionization mass spectrometry, comprising providing a probe adapted for use with a mass spectrometer comprising an adsorbent attached thereto; contacting the subject sample with the adsorbent; desorbing and ionizing the biomarker or biomarkers from the probe; and detecting the desorbed/ionized markers with the mass spectrometer.

In one embodiment, least one or more protein biomarkers are detected using immunoassays.

In one embodiment, the sample from the subject is one or more of colon biopsy material, intestinal biopsy material, fecal material, blood, blood plasma, serum, urine, cells, organs, seminal fluids, bone marrow, saliva, stool, a cellular extract, a tissue sample, a tissue biopsy, and cerebrospinal fluid.

In one embodiment, the methods for qualifying inflammatory bowel disease status in a subject further comprise measuring the amount of each biomarker in the subject sample and determining the ratio of the amounts between the markers. In a related embodiment, the measuring is selected from detecting the presence or absence of the biomarkers(s), quantifying the amount of marker(s), and qualifying the type of biomarker. In one embodiment, at least two biomarkers are measured. In a related embodiment, at least three biomarkers are measured. In another embodiment, at least four biomarkers are measured. In yet another embodiment, at least one UC and at least one CD biomarker is measured.

In one embodiment, the protein biomarkers are measured by one or more of electrospray ionization mass spectrometry (ESI-MS), ESI-MS/MS, ESI-MS/(MS)ⁿ, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS).sup.n, atmospheric pressure photoionization mass spectrometry (APPI-MS), APPI-MS/MS, and APPI-(MS)ₙ, quadrupole mass spectrometry, fourier transform mass spectrometry (FTMS), and ion trap mass spectrometry, where n is an integer greater than zero.

In one embodiment, the correlating is performed by a software classification algorithm.

The invention provides kits, for example, for aiding the diagnosis of inflammatory bowel disease or the diagnosis of the subtypes of inflammatory bowel disease. The kits may suitably include an adsorbent, wherein the adsorbent retains one or more biomarkers selected from one or more of the markers of Tables 1-9, and written instructions for use of the kit for detection of inflammatory bowel disease.

In one embodiment, the kit for aiding the diagnosis of the subtypes of inflammatory bowel disease, comprises an adsorbent, wherein the adsorbent retains one or more biomarkers selected from each of Markers 1-48 and Markers 49-75, and written instructions for use of the kit for detection of the IBD or a subtype of inflammatory bowel disease, e.g., UC or CD.

Kits may also comprise instructions provide for contacting a test sample with the adsorbent and detecting one or more biomarkers retained by the adsorbent, wherein the adsorbent is, for example, an antibody, single or double stranded oligonucleotide, amino acid, protein, peptide or fragments thereof.

In one embodiment, the one or more protein biomarkers is detected using mass spectrometry, immunoassays, or PCR. In another embodiment, the measuring is selected from detecting the presence or absence of the biomarkers(s), quantifying the amount of marker(s), and qualifying the type of biomarker.

In one aspect, the invention provides methods for identifying a candidate compound for treating inflammatory bowel disease comprising contacting one or more of the biomarkers of Tables 1-9 with a test compound; and determining whether the test compound interacts with the biomarker, wherein a compound that interacts with the biomarker is identified as a candidate compound for treating inflammatory bowel disease.

The invention also provides methods of treating inflammatory bowel disease comprising administering to a subject suffering from or at risk of developing inflammatory bowel disease a therapeutically effective amount of a compound capable of modulating the expression or activity of one or more of the biomarkers of Tables 1-9. In another aspect, the invention provides methods of treating a condition in a subject comprising administering to a subject a therapeutically effective amount of a compound which modulates the expression or activity of one or more of the biomarkers of Tables 1-9.

In certain embodiments, the compound are selected from the group consisting of enzyme inhibitor, cytotoxic drug, cytokin, chemokine, antibodies, a DNA molecule, an RNA molecule, a small molecule, a peptide, and a peptidomimetic. Classes of drugs include, anti-inflammatory, antibiotic, antiviral, antidepressant, anticonvulsant therapeutics.

According to one aspect, the invention provides methods for modulating the concentration of a biomarker, wherein the biomarker is one or more of the biomarkers listed in Tables 1-9. The method comprises contacting a cell with a test compound, measuring at least one biomarker, wherein the biomarker is selected from one or more of the biomarkers of Tables 1-9, and correlating the measurement with a determination of efficacy.

The invention also provides, in one aspect, a method of identifying a biomarker comprising obtaining an endoscopic sample from a subject, isolating nucleic acid from the sample, analyzing the nucleic acid and correlating the results. The results may be analyzed against a control database of IBD samples and/or controls.

The invention also provides methods of determining the inflammatory bowel disease status of a subject, comprising (a) obtaining a biomarker profile from a sample taken from the subject; and (b) comparing the subject's biomarker profile to a reference biomarker profile obtained from a reference population, wherein the comparison is capable of classifying the subject as belonging to or not belonging to the reference population; wherein the subject's biomarker profile and the reference biomarker profile comprise one or more markers listed in Tables 1-9.

In one embodiment, the comparison of the biomarker profiles can determine inflammatory bowel disease status in the subject with an accuracy of at least about 60%, 70%, 80%, 90% or approaching 100%.

In certain embodiments, the sample is fractionated by one or more of chemical extraction partitioning, ion exchange chromatography, reverse phase liquid chromatography, isoelectric focusing, one-dimensional polyacrylamide gel electrophoresis (PAGE), two-dimensional polyacrylamide gel electrophoresis (2D-PAGE), thin-layer chromatography, gas chromatography, liquid chromatography, and any combination thereof.

In other methods, the measuring step comprises quantifying the amount of marker(s) in the sample. In other methods, the measuring step comprises qualifying the type of biomarker in the sample.

When the identity of a markers is not yet known, the biomarkers may be sufficiently characterized by, e.g., mass and by affinity characteristics. It is noted that molecular weight and binding properties are characteristic properties of the markers and not limitations on means of detection or isolation. Furthermore, using the methods described herein or other methods known in the art, the absolute identity of markers can be determined.

The present invention also relates to biomarkers designated as Markers 1-97, 99-211, 213-264, 266-401. Protein markers of the invention can be characterized in one or more of several respects. In particular, in one aspect, these markers are characterized by molecular weights under the conditions specified herein, particularly as determined by mass spectral analysis. In another aspect, the markers can be characterized by features of the markers' mass spectral signature such as size (including area) and/or shape of the markers' spectral peaks, features including proximity, size and shape of neighboring peaks, etc. In yet another aspect, the markers can be characterized by affinity binding characteristics, particularly ability to binding to cation-exchange and/or hydrophobic surfaces. In preferred embodiments, markers of the invention may be characterized by each of such aspects, i.e. molecular weight, mass spectral signature and cation and/or hydrophobic absorbent binding.

Accuracy and resolution variances associated with the techniques described herein are reflected in the use of the term “about” in the disclosure.

In a preferred embodiment, the present invention provides for a method for detecting and diagnosing (including e.g., differentiating between) different subtypes of inflammatory bowel disease, wherein the method comprises using a biochip array for detecting at least one biomarker in a subject sample; evaluating at least one biomarker in a subject sample, and correlating the detection of one or more protein biomarkers with a inflammatory bowel disease subtype, e.g., UC and CD.

The biomarkers of the invention may be detected in samples of blood, blood plasma, serum, urine, tissue, cells, organs, seminal fluids, bone marrow, colon biopsies, intestinal biopsies, and cerebrospinal fluid.

Preferred detection methods include use of a biochip array. Biochip arrays useful in the invention include protein and nucleic acid arrays. One or more markers are captured on the biochip array and subjected to laser ionization to detect the molecular weight of the markers. Analysis of the markers is, for example, by molecular weight of the one or more markers against a threshold intensity that is normalized against total ion current.

In preferred methods of the present invention, the step of correlating the measurement of the biomarkers with inflammatory bowel disease status is performed by a software classification algorithm. Preferably, data is generated on immobilized subject samples on a biochip array, by subjecting the biochip array to laser ionization and detecting intensity of signal for mass/charge ratio; and transforming the data into computer readable form; and executing an algorithm that classifies the data according to user input parameters, for detecting signals that represent markers present in inflammatory bowel disease subjects and are lacking in non-inflammatory bowel disease subject controls.

Preferably the biochip surfaces are, for example, ionic, anionic, hydrophobic; comprised of immobilized nickel or copper ions, comprised of a mixture of positive and negative ions; and/or comprised of one or more antibodies, single or double stranded nucleic acids, proteins, peptides or fragments thereof, amino acid probes, or phage display libraries.

In other preferred methods one or more of the markers are measured using laser desorption/ionization mass spectrometry, comprising providing a probe adapted for use with a mass spectrometer comprising an adsorbent attached thereto, and contacting the subject sample with the adsorbent, and desorbing and ionizing the marker or markers from the probe and detecting the deionized/ionized markers with the mass spectrometer.

Preferably, the laser desorption/ionization mass spectrometry comprises: providing a substrate comprising an adsorbent attached thereto; contacting the subject sample with the adsorbent; placing the substrate on a probe adapted for use with a mass spectrometer comprising an adsorbent attached thereto; and desorbing and ionizing the marker or markers from the probe and detecting the desorbed/ionized marker or markers with the mass spectrometer.

The adsorbent can for example be, hydrophobic, hydrophilic, ionic or metal chelate adsorbent, such as nickel or copper, or an antibody, single- or double stranded oligonucleotide, amino acid, protein, peptide or fragments thereof.

In another embodiment, a process for purification of a biomarker, comprising fractioning a sample comprising one or more protein biomarkers by size-exclusion chromatography and collecting a fraction that includes the one or more biomarker; and/or fractionating a sample comprising the one or more biomarkers by anion exchange chromatography and collecting a fraction that includes the one or more biomarkers. Fractionation is monitored for purity on normal phase and immobilized nickel arrays. Generating data on immobilized marker fractions on an array is accomplished by subjecting the array to laser ionization and detecting intensity of signal for mass/charge ratio; and transforming the data into computer readable form; and executing an algorithm that classifies the data according to user input parameters, for detecting signals that represent markers present in inflammatory bowel disease subjects and are lacking in non-inflammatory bowel disease subject controls. Preferably fractions are subjected to gel electrophoresis and correlated with data generated by mass spectrometry. In one aspect, gel bands representative of potential markers are excised and subjected to enzymatic treatment and are applied to biochip arrays for peptide mapping.

In another aspect one or more biomarkers are selected from gel bands representing Markers 1-97, 99-211, 213-264, 266-401 described herein.

Purified proteins for detection of inflammatory bowel disease and/or screening and aiding in the diagnosis of inflammatory bowel disease and/or generation of antibodies for further diagnostic assays are provided.

In further embodiments, the invention provides methods for identifying compounds (e.g., antibodies, nucleic acid molecules (e.g., DNA, RNA), small molecules, peptides, and/or peptidomimetics) capable of treating inflammatory bowel disease comprising contacting at least one or more of a biomarker selected from Markers 1-97, 99-211, 213-264, 266-401, and combinations thereof with a test compound; and determining whether the test compound interacts with, binds to, or modulates the biomarker, wherein a compound that interacts with, binds to, or modulates the biomarker is identifies as a compound capable of treated inflammatory bowel disease.

In another embodiment, the invention provides methods of treating inflammatory bowel disease comprising administering to a subject suffering from or at risk of developing inflammatory bowel disease a therapeutically effective amount of a compound (e.g., an antibody, nucleic acid molecule (e.g., DNA, RNA), small molecule, peptide, and/or peptidomimetic) capable of modulating the expression or activity of one or more of the Biomarkes 1-75.

In one aspect, the invention provides methods of determining the inflammatory bowel disease status of a subject, comprising (a) obtaining a biomarker profile from a sample taken from the subject; and (b) comparing the subject's biomarker profile to a reference biomarker profile obtained from a reference population, wherein the comparison is capable of classifying the subject as belonging to or not belonging to the reference population; wherein the subject's biomarker profile and the reference biomarker profile comprise one or more markers listed in Tables 1-9.

Methods of the invention, one embodiment, may further comprise repeating the method at least once, wherein the subject's biomarker profile is obtained from a separate sample taken each time the method is repeated.

In another embodiment, samples from the subject are taken about 24, 30, 48, 60, and/or 72 hours apart.

In another embodiment, the comparison of the biomarker profiles can determine inflammatory bowel disease status in the subject with an accuracy of at least about 60% to about 99%.

In one embodiment, the reference biomarker profile is obtained from a population comprising a single subject, at least two subjects, and at least 20 subjects.

Thus, the methods of the present invention provide and solve the need for methods of accurately assessing, i.e., diagnostically, prognostically, and therapeutically, IBD, including UC and CD.

Other embodiments of the invention are disclosed infra.

### BRIEF DESCRIPTION OF THE DRAWINGS

FIG. 1 depicts gene expression signals from CD-76-aff-1 (X axis) and CD-76-aff-2 (Y axis) biopsies from one affected area. Each point represents the expression value of a probe set (defining a gene) in log-scale in the two biopsies. A probe set with a “Present” call in both arrays (red), “Absent” in both (yellow), and “Present” in either one of the two arrays (blue) is shown. The diagonal lines indicate fold change of 2, 3, and 10 in expression levels between two arrays. For genes expressed differentially between the two arrays, change in expression must be ≧2 fold, expression ≧100 arbitrary units, and “Present call” in one sample.

FIG. 2 depicts multidimensional scaling (MDS) of 32 samples. In a four dimensional representation of the data we compared the dimensions in a pair-wise fashion. A plot of component1 versus component 2 is shown, divided into four quadrants (Q1-Q4). Healthy controls: black open circles, CD affected: solid blue triangles, CD unaffected: open blue triangle, UC affected: red solid square, UC unaffected: open red square. Each affected is linked to its corresponding unaffected sample by a line. The affected IBD biopsies fall primarily in Q 1 and Q4, normal and several unaffected CD appear in Q2 and Q3, with unaffected UC biopsy profiles localizing to Q3.

FIG. 3 depicts hierarchical clustering across all arrays, of the top 50 genes whose expression patterns correlate with the distribution of samples in the MDS plot of FIG. 2. The inflammation score (*) for each biopsy taken from Tables 1-9 are shown on the top. Genes with similar expression levels across samples are clustered vertically and samples with similar gene expression patterns are grouped horizontally. Genes expressed above mean (red), mean (black) and below mean (green) are as shown. To derive this set of genes, each sample was assigned to one of four groups, depending on which quadrant it occupied in the MDS map, and an analysis of variance (ANOVA) on the expression values for each gene was calculated. Genes with large F-statistics have strong quadrant specific differences in expression. The top 50 genes with the highest F-statistic scores are shown.

FIG. 4 is a model showing distinct pathogenic events in UC and CD. Gene symbols are taken from Tables 2, 3 and FIG. 4. Gene up regulations and down regulations are indicated by arrows. We speculate that in response to microbial and other environmental stimuli, CD shows a deregulated immune response that entails acute phase response, antigen presentation and macrophage activation. In contrast early events in UC suggest impaired detoxification, overload of unfolded proteins and endoplasmic reticulum stress.

FIG. 5 depicts histology of endoscopic biopsies of colon from a healthy control (A), CD-76, a patient with Crohn's disease (B and C), and UC-55, a patient with ulcerative colitis (D). (B) is taken from unaffected mucosa showing essentially normal colon structures. (C), a view of CD76 affected biopsy, showing significant inflammatory infiltration in the mucosa and submucosa, cryptitis with crypt abscesses, and basal lymphoplasmacytosis (inflammation grade: ++). (D), UC-55 affected demonstrates crypt distortion and dropout, and lamina propria fibrosis (fibrosis grade: ++). MM: muscularis mucosa (*), SM: submucosa. H&E staining, original magnification 40×.

FIG. 6 depicts the expressions of selected genes that were quantified by real-time RT-PCR. The relative expression value of a gene was normalized to that of GAPD. The samples include unaffected (un) and affected (aff) sample from six CD cases (CD-33, 51, 53, 58, 59 and 76), five UC samples (UC-32, 35, 38, 44 and 55) and four from normal controls (N65, N66, N69 and N79). Each point represents an individual sample. Gene symbols are CXCL1: chemokine (C-X-C motif) ligand 1, DMBT1: deleted in malignant brain tumors 1, ADM: adrenomedullin, STAT3: signal transducer and activator of transcription 3, ASMT: acetylserotonin 0-methyltransferase, IFI35: interferon-induced protein 35, PSME2: proteasome activator subunit 2, and PSMB8: proteasome subunit, beta type, 8. The horizontal bar indicates the mean value of each group.

FIG. 7 depicts multidimensional scaling (MDS) of 36 samples. In a four dimensional representation of the data we compared the dimensions (components) in a pair-wise fashion. (A): A plot of component 1 versus component 2 is shown, divided into four quadrants (Q1-Q4). Solid symbols: disease affected samples, open symbols: unaffected, asterisk: healthy controls. Each “affected” sample is linked to its corresponding “unaffected” sample by a line. The “affected” samples fall primarily in Q 1 and Q4, while the “unaffected” and healthy controls appear in Q2 and Q3. (B): A plot of component 2 versus component 3 is shown. The disease-affected biopsies appear on left of the vertical line (Q 1 and Q2), the unaffected and healthy samples appear on the right side of vertical line. The two acute infectious colitis (INF156, INF157) samples appear together and separate from the CD and UC samples.

FIG. 8 shows confirmation of upregulation of selected genes by quantitative real-time RT-PCR. The genes were randomly selected from Table 2 and 3, including PSME2 (proteasome activator subunit 2), PSMB8 (proteasome subunit, beta type 8), ADM (adrenomedullin), STAT3 (signal transducer and activator of transcription 3), DMBT1 (deleted in malignant brain tumors 1) and CXCL1 (chemokine C-X-C-motif ligand 1). The relative expression value of a gene was normalized to that of GAPDH. The samples include unaffected (un) and affected (aff) sample from six CD cases (CD-33, 49, 51, 53, 58 and 76), four UC samples (UC-32, 35, 38 and 55) and four controls from healthy individuals (N65, N66, N69 and N79). Each point represents an individual sample. The horizontal bar indicates the mean value for each group.

FIG. 9 shows hierarchical clustering of genes differentially expressed in IBD and acute infectious colitis. (A) Gene expression differences between CD affected and bacterial infectious colitis (INF-156 and -157) samples were identified using the SAM software (Methods). (B) Genes differentially expressed in UC affected and bacterial infectious colitis samples are showed Genes expressed above mean (red), mean (black) and below mean (green) are as shown.

FIG. 10 shows hierarchical clustering of genes differentially expressed in IBD unaffected and healthy control samples, using SAM software (Methods). The expression patterns of these genes in CD and UC unaffected samples, and normal controls are shown as above mean (red), mean (black) and below mean (green) are as shown. Gene symbols in colored boxes indicate genes that were also identified by SAM as down-regulated in CD and/or UC affected tissue (blue: in both CD and UC, yellow: in CD, brown: in UC).

FIG. 11 shows histology of endoscopic biopsies of colon from a healthy control (A), CD-76, a patient with Crohn's disease (B and C), and UC-55, a patient with ulcerative colitis (D). (B) is taken from unaffected mucosa showing essentially normal colon structures. (C), a view of CD76 affected biopsy, showing significant inflammatory infiltration in the mucosa and submucosa, cryptitis with crypt abscesses, and basallymphoplasmacytosis (inflammation grade: ++). (D), UC-55 affected demonstrates crypt distortion and dropout, and laminapropriafibrosis (fibrosis grade: ++). MM: muscularis mucosa(*), SM: submucosa. H&E staining, original magnification 40×.

FIG. 12 shows gene expression signals from CD-76-aff-1 (X axis) and CD-76-aff-2 (Y axis) biopsies from one affected area. Each point represents the expression value of a probe set (defining a gene) in log-scale in the two biopsies. A probe set with a “Present” call in both arrays (red), “Absent” in both (yellow), and “Present” in either one of the two arrays (blue) is shown. The diagonal lines indicate fold change of 2, 3, and 10 in expression levels between two arrays. For genes expressed differentially between the two arrays, change in expression must be ≧2 fold, expression³ 100 arbitrary units, and “Present call” in one sample.

FIG. 13 shows expression of TAP1 (transporter 1, ATP-binding cassette, sub-family B) protein incolonicbiopsies. TAP1 protein was detected by immunohistochemistry using a polyclonal anti-human TAP1 antibody. Representative views are shown for colonoscopic biopsies taken from a healthy control (A), two patients with CD (B and C), and a patient with UC (D). Detail on patient demographics is included in supplementary Table s2. Positive reaction (brown) is seen in cytoplasma of mononuclear cells (arrows) and some epithelial cells (arrowheads).

FIG. 14—depicts unsupervised cluster analysis by multidimensional scaling. In a four dimensional representation of the data we compared the dimensions (components) in a pair-wise fashion. (A): A plot of component 1 versus component 2 is shown, divided into four quadrants (Q1-Q4). CD: Crohn's disease, UC: ulcerative colitis, INF: infectious colitis, IC: indeterminate colitis, number next to each code denotes specific cases, detailed information for which are in Table 1. Solid symbols: disease affected samples, open symbols: unaffected, asterisk: healthy controls. Each “affected” sample is linked to its corresponding “unaffected” sample by a line. In general “affected” samples appear to separate along component 2 axis, placed in Q 1 and Q4, while the “unaffected” and healthy controls appear below the horizontal axis, in Q2 and Q3. (B): A plot of component 2 versus component 3 is shown. The disease-affected biopsies appear on left of the vertical line (Q 1 and Q2), the unaffected and healthy samples appear on the right side of vertical line. The two acute infectious colitis (INF156, INF157) samples appear together and separated from CD and UC. (C): Site of biopsy is shown for each sample in the first component 2 versus component1 plot. Rm: rectum, S: sigmoid colon, DC: descending colon, SF: splenic flexure, TC: transverse colon, HF: hepatic flexure, AC: ascending colon, Ce: cecum.

FIG. 15 depicts heat image of all differentially expressed genes in IBD and INF compared to normal controls. Green denotes below mean (black) and red above mean relative gene expression. Details on genes differentially expressed in CD, CD+UC and UC compared to normal are shown in Tables 4, 5 and 6.

FIG. 16 depicts confirmation of selected up regulated genes by quantitative real-time RTPCR. The genes were randomly selected from Table 4-6 and include PSME2 (proteasome activator subunit 2), PSMB8 (proteasome subunit, beta type 8), ADM (adrenomedullin), STAT3 (signal transducer and activator of transcription 3), DMBT1 (deleted in malignant brain tumors 1) and CXCL1 (chemokine C-X-C-motif ligand 1). The relative expression value of a gene was normalized to that of GAPDH. Total RNA from six CD affected (CD-33, 49, 51, 53, 58 and 76), four UC affected (UC-32, 35, 38 and 55) and four controls (N65, N66, N69 and N79) were used to measure selected gene transcripts. The relative expression of a gene in each group was presented as a Box-Whiskers chart indicating the 25th and 75th percentiles of the data set with a box (a line through the box marks the 50th percentile). The data range (1 to 99 percentile) is shown by the whiskers and the black square represents the mean value for each group. *Statistical significance was determined with one-way unpaired Student's t test with p<0.05 considered statistically significant.

FIG. 17 depicts immunostaining of TAP1 (transporter 1, ATP-binding cassette, sub-family B) in colonic biopsies. TAP1 protein was detected using a polyclonal anti-human TAP1 antibody. Representative views are shown for colonoscopic biopsies taken from a healthy control (A) and two patients with CD (B and C). Positive reaction (brown) is seen in cytoplasm of mononuclear cells (arrows) and some epithelial cells (arrowhead).

FIG. 18 depicts differentially expressed genes in IBD unaffected and healthy control samples. SAM software was used to determine the gene expression patterns of CD and UC unaffected samples compared to normal controls. Relative gene expression is shown as above mean (red), mean (black) and below mean (green).

### DETAILED DESCRIPTION

The present invention provides biomarkers generated from comparison of protein profiles from subjects diagnosed with inflammatory bowel disease and from subjects without known neoplastic diseases, using the mass spectrometry techniques. In particular, the invention provides that these biomarkers, used individually, or preferably in combination with other biomarkers from this group or with other diagnostic tests, provide a novel method of determining inflammatory bowel disease status in a subject.

The present invention presents markers that are differentially present in samples of inflammatory bowel disease subjects and control subjects, and the application of this discovery in methods and kits for determining inflammatory bowel disease status. These protein markers are found in samples from inflammatory bowel disease subjects at levels that are different than the levels in samples from subject in whom human IBD is undetectable. Accordingly, the amount of one or more markers found in a test sample compared to a control, or the presence or absence of one or more markers in the test sample provides useful information regarding the inflammatory bowel disease status of the patient.

The present invention also relates to a method for identification of biomarkers for IBD, with high specificity and sensitivity. In particular, a panel of biomarkers were identified that are associated with inflammatory bowel disease status.

In the data presented herein, we describe for the first time a serum protein profile which aids in the diagnosis of inflammatory bowel disease. Examining 139 samples of subjects and healthy persons, this profile distinguished subjects with inflammatory bowel disease from control subjects independent validation sets.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Inflammatory bowel disease,” as used herein, refers to a functional disorder of the colon (large intestine) that causes crampy abdominal pain, bloating, constipation and/or diarrhea. IBS is classified as a functional gastrointestinal disorder because no structural or biochemical cause can be found to explain the symptoms. The most common symptoms of IBD include, abdominal pain, weight loss, fever, rectal bleeding, skin and eye irritations, and diarrhea. Intervals of active disease, or ‘flares’, and periods of remission characterize IBD. Upon diagnostic testing, the colon shows no evidence of disease such as ulcers or inflammation. Therefore, IBS preferably diagnosed only after other possible digestive disorders and diseases have been ruled out. IBS is often misdiagnosed or misnamed as colitis, mucous colitis, spastic colon, irritable bowel disease or spastic bowel (colon).

“Ulcerative colitis,” as used herein refers to a disease that is a form of IBD and causes inflammation and sores, called ulcers, in the top layers of the lining of the large intestine. Common symptoms of UC include bloody diarrhea, fever and abdominal pain. There can also be symptoms outside the digestive system which are known as extra-intestinal symptoms. Fever is a characteristic of the inflammatory process that takes place in UC and there are several extra-intestinal symptoms that are not directly related to the inflammation in the colon and include eye inflammation, joint pains, skin rashes or lesions, and mouth ulcers. UC is diagnosed, for example, by stool culture, blood tests, fecal occult blood test, sigmoidoscopy, colonoscopy, and barium enema. There are several types of medications that are frequently used to treat UC, including, for example, sulfasalazine (Azulfadine), mesalamine (Asacol, Pentasa, Rowasa), osalazine (Dipentum), (Colazal) and corticosteroids (prednisone). Surgery may also be used to treat UC, usually after all available drug treatments have failed. Surgery for UC always involves a total colectomy, or a complete removal of the large intestine (colon). Resection, or removing only the diseased section of the colon, is not an option in UC, because the disease will only re-occur in the portion of the colon that is left.

“Crohn's disease,” as used herein refers to a form of IBD that is manifested by inflammation anywhere along the digestive tract from the mouth to the anus. Of CD cases, 45% occur in ileum and colon, 35% in just the ileum, and 20% in just the colon. Unlike ulcerative colitis (UC), which only affects the inner layer, CD commonly involves all layers of the intestinal wall. Common symptoms of CD include chronic diarrhea fever, abdominal pain, weight loss, and lack of appetite. Frequent diarrhea can lead to dehydration and nutritional deficiencies. Because the colon is inflamed, it is not as efficient at absorbing water and nutrients from food. Other symptoms include, fistulas and fissures. A fissure is a tear or ulcer in the lining of the anal canal and symptoms include painful bowel movements, bright red blood in toilet bowel or on paper, anal lump, and swollen skin tag. Acute fissures may be treated with Sitz baths, fiber to create softer stools, stool softeners, topical hydrocortisone, zinc oxide, petroleum jelly and topical anesthetics. A chronic fissure may need more aggressive treatment including surgery. A fistula is an abnormal tunnel connecting two body cavities or a body cavity to the skin. Approximately 30% of people with Crohn's Disease develop fistulas. Treatments include antibiotics, immunosuppressants, Remicade, liquid nutrition to replace solid food and surgery. Treatments for CD include, for example, sulfasalazine (Azulfadine), mesalamine (Asacol, Pentasa), balsalazide disodium (Colazal®), azathioprine (Imuran), 6-MP (Purinethol), cyclosporine, methotrexate, infliximab (Remicade), rifaximin, Budesonide (Entocort EC), and corticosteroids (prednisone). Surgery may also be used to treat CD, including resection, ileostomy, stoma, and strictureplasty, usually after all available drug treatments have failed. Anywhere from 40 to 60% of CD patients who have disease in the small bowel will have surgery in the first 10 years after diagnosis. Several different types of surgery are used to treat symptoms and complications of CD, yet none are a cure. Several tests may be used by physicians to diagnose CD, including, barium enema, upper GI series, stool culture, blood tests to determine a white blood cell count or if anemia is present, fecal occult blood test, sigmoidoscopy, colonoscopy, and other tests may be used to rule out other potential diagnoses.

The term “inflammatory bowel disease status” refers to the status of the disease in the patient. Examples of types of inflammatory bowel disease statuses include, but are not limited to, the subject's risk of IBD, including colorectal UC or CD, the presence or absence of disease (e.g., IBD, UC or CD), the stage of disease in a patient (e.g., IBD, UC or CD), and the effectiveness of treatment of disease. Other statuses and degrees of each status are known in the art.

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

“Laser desorption mass spectrometer” refers to a mass spectrometer that uses laser energy as a means to desorb, volatilize, and ionize an analyte.

“Tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector—magnetic sector mass spectrometers, and combinations thereof.

“Mass analyzer” refers to a sub-assembly of a mass spectrometer that comprises means for measuring a parameter that can be translated into mass-to-charge ratios of gas phase ions. In a time-of-flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the energy delivered per unit area of interrogated image. A high fluence source, such as a laser, will deliver about 1 mJ/mm2 to 50 mJ/mm2. Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Solid support” refers to a solid material which can be derivatized with, or otherwise attached to, a capture reagent. Exemplary solid supports include probes, microtiter plates and chromatographic resins.

“Probe” in the context of this invention refers to a device adapted to engage a probe interface of a gas phase ion spectrometer (e.g., a mass spectrometer) and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

“Surface-enhanced laser desorption/ionization” or “SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. No. 5,719,060 (Hutchens and Yip) and U.S. Pat. No. 6,225,047 (Hutchens and Yip).

“Surface-Enhanced Affinity Capture” or “SEAC” is a version of SELDI that involves the use of probes comprising an absorbent surface (a “SEAC probe”). “Adsorbent surface” refers to a surface to which is bound an adsorbent (also called a “capture reagent” or an “affinity reagent”). An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

“Adsorption” refers to detectable non-covalent binding of an analyte to an adsorbent or capture reagent.

“Surface-Enhanced Neat Desorption” or “SEND” is a version of SELDI that involves the use of probes comprising energy absorbing molecules chemically bound to the probe surface. (“SEND probe.”) “Energy absorbing molecules” (“EAM”) refer to molecules that are capable of absorbing energy from a laser desorption/ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. SEND is further described in U.S. Pat. No. 5,719,060 and U.S. patent application 60/408,255, filed Sep. 4, 2002 (Kitagawa, “Monomers And Polymers Having Energy Absorbing Moieties Of Use In Desorption/Ionization Of Analytes”).

“Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light, e.g., laser light. SEPAR is further described in U.S. Pat. No. 5,719,060.

“Eluant” or “wash solution” refers to an agent, typically a solution, which is used to affect or modify adsorption of an analyte to an adsorbent surface and/or remove unbound materials from the surface. The elution characteristics of an eluant can depend on, for example, pH, ionic strength, hydrophobicity, degree of chaotropism, detergent strength and temperature.

“Analyte” refers to any component of a sample that is desired to be detected. The term can refer to a single component or a plurality of components in the sample.

The “complexity” of a sample adsorbed to an adsorption surface of an affinity capture probe means the number of different protein species that are adsorbed.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Biochip” refers to a solid substrate having a generally planar surface to which an adsorbent is attached. Frequently, the surface of the biochip comprises a plurality of addressable locations, each of which location has the adsorbent bound there. Biochips can be adapted to engage a probe interface, and therefore, function as probes.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000). Protein biochips produced by Ciphergen Biosystems comprise surfaces having chromatographic or biospecific adsorbents attached thereto at addressable locations. Biochips are further described in: WO 00/66265 (Rich et al., “Probes for a Gas Phase Ion Spectrometer,” Nov. 9, 2000); WO 00/67293 (Beecher et al., “Sample Holder with Hydrophobic Coating for Gas Phase Mass Spectrometer,” Nov. 9, 2000); U.S. patent application US20030032043A1 (Pohl and Papanu, “Latex Based Adsorbent Chip,” Jul. 16, 2002) and U.S. patent application 60/350,110 (Um et al., “Hydrophobic Surface Chip,” Nov. 8, 2001).

Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry, and in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

“Marker” or “biomarker” in the context of the present invention refer to a polypeptide (of a particular apparent molecular weight) or nucleic acid, which is differentially present in a sample taken from subjects having human inflammatory bowel disease as compared to a comparable sample taken from control subjects (e.g., a person with a negative diagnosis or undetectable inflammatory bowel disease, normal or healthy subject). The term “biomarker” is used interchangeably with the term “marker.” The biomarkers are identified by molecular mass in Daltons, and include the masses centered around the identified molecular masses for each marker.

The term “measuring” means methods which include detecting the presence or absence of marker(s) in the sample, quantifying the amount of marker(s) in the sample, and/or qualifying the type of biomarker. Measuring can be accomplished by methods known in the art and those further described herein, including but not limited to microarray analysis (with Significance Analysis of Microarrays (SAM) software), SELDI and immunoassay. Any suitable methods can be used to detect and measure one or more of the markers described herein. These methods include, without limitation, mass spectrometry (e.g., laser desorption/ionization mass spectrometry), fluorescence (e.g. sandwich immunoassay), surface plasmon resonance, ellipsometry and atomic force microscopy.

“Detect” refers to identifying the presence, absence or amount of the object to be detected.

The phrase “differentially present” refers to differences in the quantity and/or the frequency of a marker present in a sample taken from subjects having human IBD as compared to a control subject. For example, some markers described herein are present at an elevated level in samples of subjects compared to samples from control subjects. In contrast, other markers described herein are present at a decreased level in samples of inflammatory bowel disease subjects compared to samples from control subjects. Furthermore, a marker can be a polypeptide, which is detected at a higher frequency or at a lower frequency in samples of human IBD subjects compared to samples of control subjects.

Furthermore, a marker can be a polypeptide, which is detected at a higher frequency or at a lower frequency in samples of unaffected tissue from human IBD subjects compared to samples affected tissue from human IBD subjects.

Furthermore, a marker can be a polypeptide, which is detected at a higher frequency or at a lower frequency in samples of human unaffected tissue from IBD subjects compared to samples of control subjects.

Furthermore, a marker can be a polypeptide, which is detected at a higher frequency or at a lower frequency in samples of human affected tissue from IBD subjects compared to samples of control subjects.

A marker can be differentially present in terms of quantity, frequency or both.

“Affected tissue,” as used herein refers to tissue from and IBD subject that is grossly diseased tissue (tissue that is inflamed or shows fibrosis.

“Unaffected tissue,” as used herein refers to a tissue from an IBD subject that is from a portion of tissue that does not have gross disease present, for example tissue that is about 1, 2, 5, 10, 20 or more cm from grossly diseased tissue.

A polypeptide is differentially present between two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between two sets of samples if the frequency of detecting the polypeptide in the IBD subjects' samples is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition, i.e., inflammatory bowel disease. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of inflammatory bowel disease. A diagnostic amount can be either in absolute amount (e.g., μg/ml) or a relative amount (e.g., relative intensity of signals).

A “control amount” of a marker can be any amount or a range of amount, which is to be compared against a test amount of a marker. For example, a control amount of a marker can be the amount of a marker in a person without inflammatory bowel disease. A control amount can be either in absolute amount (e.g., μg/ml) or a relative amount (e.g., relative intensity of signals).

As used herein, the term “sensitivity” is the percentage of subjects with a particular disease. For example, in the inflammatory bowel disease group, the biomarkers of the invention have a sensitivity of about 80.0%-98.6%, and preferably a sensitivity of 85%, 87.5%, 90%, 92.5%, 95%, 97%, 98%, 99% or approaching 100%.

As used herein, the term “specificity” is the percentage of subjects correctly identified as having a particular disease i.e., normal or healthy subjects. For example, the specificity is calculated as the number of subjects with a particular disease as compared to non-IBD subjects (e.g., normal healthy subjects). The specificity of the assays described herein may range from about 80% to 100%. Preferably the specificity is about 90%, 95%, or 100%.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

“Immunoassay” is an assay that uses an antibody to specifically bind an antigen (e.g., a marker). The immunoassay is characterized by the use of specific binding properties of a particular antibody to isolate, target, and/or quantify the antigen.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well-characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker “X” from specific species such as rat, mouse, or human can be selected to obtain only those polyclonal antibodies that are specifically immunoreactive with marker “X” and not with other proteins, except for polymorphic variants and alleles of marker “X”. This selection may be achieved by subtracting out antibodies that cross-react with marker “X” molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Managing subject treatment” refers to the behavior of the clinician or physician subsequent to the determination of IBD status. For example, if the result of the methods of the present invention is inconclusive or there is reason that confirmation of status is necessary, the physician may order more tests. Alternatively, if the status indicates that treatment is appropriate, the physician may schedule the patient for treatment, e.g., surgery, administer one or more therapeutic agents or radiation. Likewise, if the status is negative, e.g., late stage inflammatory bowel disease or if the status is acute, no further action may be warranted. Furthermore, if the results show that treatment has been successful, a maintenance therapy or no further management may be necessary.

### Description of the Biomarkers

Crohn's Disease Biomarkers

CD biomarkers include the proteins or their encoding nucleic acids for the following pathways or cellular processes: acute phase and innate immune response (IL-1 and TNFα mediated induction of NF-κB), immune response, apoptosis, inflammatory cell recruitment pathways, inflammatory response (IL1B, S100A8), antigen presentation (MHC class II immunoproteasome members PSME2 and PSMB8, MHC class II ATP-binding antigen peptide transporter TAP1, HLA-DMA and UBD of MHC class I), inflammatory cell chemotaxis (IL8, CXCL1, CXCL3), apoptosis (CASP1, CASP10), macrophage activation (ASMT and interferon-regulated genes IFITM1, IFITM3, ISG20, IFI35, SP110), leukocyte protection (LILRB encoding a receptor for class I MHC antigens), recruitment of inflammatory cells, acute phase response (ADM, STAT1, STAT3, and protease inhibitors SERPINA1 and SPINK1 to prevent tissue destruction), and chemokine and interferon-γ responsive genes.

Crohn's disease patients often require surgery due to obstruction, when disease may be well established and gene expression patterns rather static. Profiling of endoscopic biopsies provide the opportunity to interrogate all stages of disease. Secondly, since only a fraction of IBD patients require surgery, large numbers of IBD cases remain unexplored. Clinical sub grouping of CD is based on anatomic site of involvement (ileum only, colon only, or upper small bowel and colon)¹² and disease behavior (inflammatory, stricturing, or fistulizing).^{13, 14}

Pinch biopsies are collected during endoscopy for routine evaluation of disease activity by histology¹⁵ To further develop the methods of the invention, single endoscopic pinch biopsies were used from nine colonic Crohn's disease cases with mild to severe inflammation, five ulcerative colitis cases and four healthy controls. For each IBD case, expression patterns for a biopsy from an affected and one from an unaffected area (as judged during endoscopy) were obtained. Multidimensional scaling of the expression patterns distinguished IBD from healthy individuals, CD from UC, and also unaffected from healthy controls. Although, Crohn's colitis harbors some phenotypic overlaps with ulcerative colitis, the expression profiles identify a distinct set of differentially expressed genes, and distinct pathophysiologies, for each disease.

### UC Biomarkers

UC biomarkers include the proteins or their encoding nucleic acids for the following pathways or cellular processes: endoplasmic reticulum stress pathway members, protein-trafficking pathway members, and detoxification and cell growth pathway members.

Further UC biomarkers include the proteins or their encoding nucleic acids for the following pathways or cellular processes: up-regulations of complement cascade activation (BF and C4A), growth regulatory (MIA) and apoptosis (ATM) pathways, detoxification (NNMT) and intracellular transport (SNX26) pathways; and down regulations of biosynthetic and metabolic processes (PANK3, HPGD), and endoplasmic reticulum-, Golgi-transport/intracellular trafficking (F2RL1, GABRG3, GNGT1, SLC4A4).

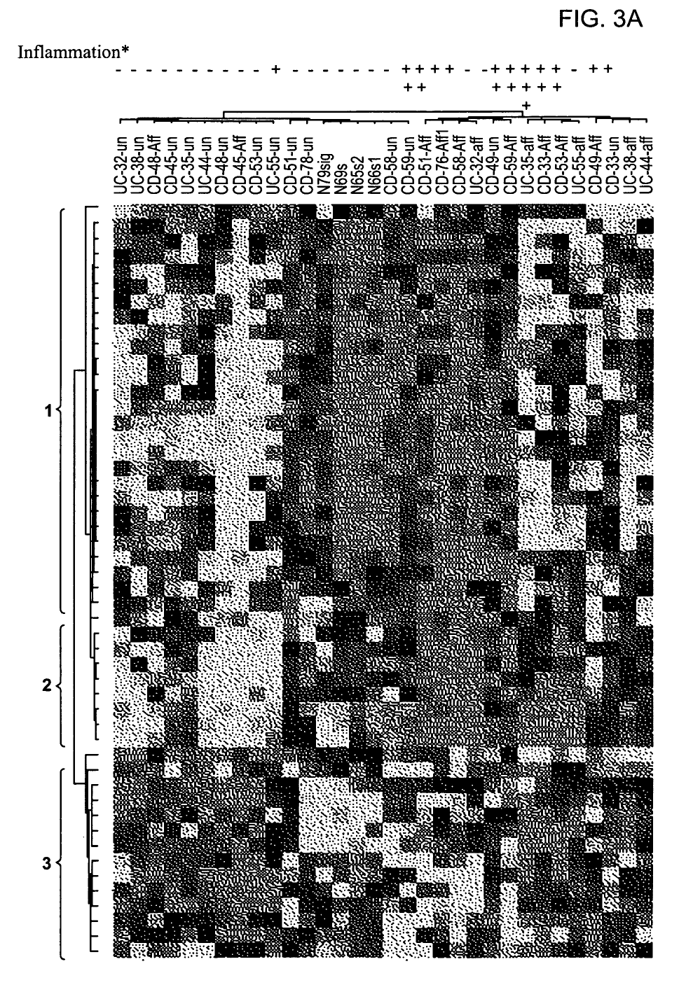

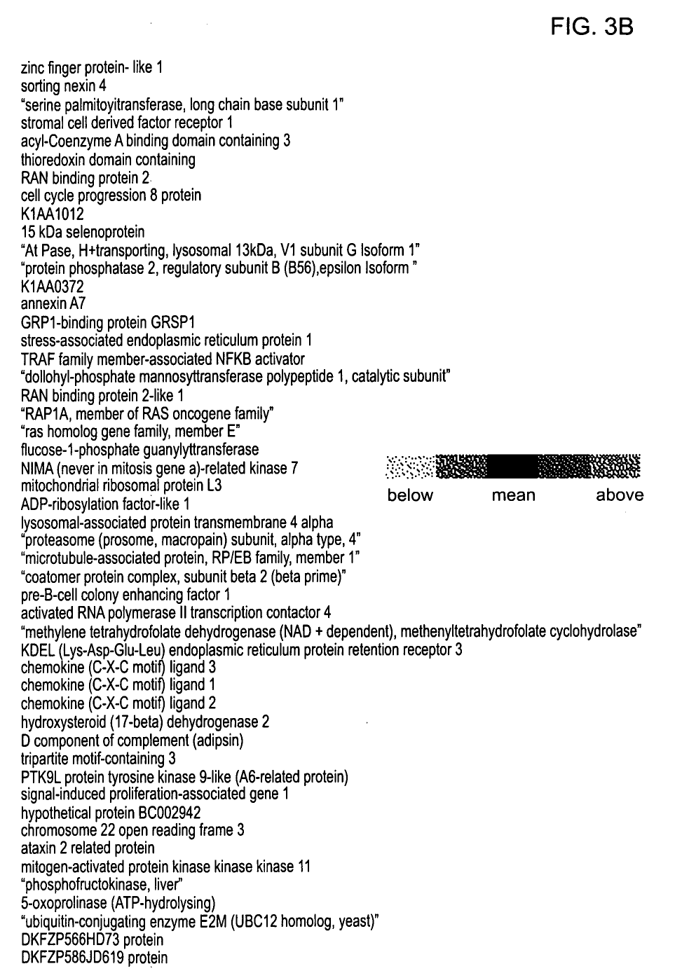

Thirteen genes are over expressed in UC primarily and the two UC-like CD cases 33 and 53, roughly distinguishing UC from CD (FIG. 3).

Resection of tissued shows different gene expression patterns than does biopsy of tissue. For example, UC patterns are quite dynamic showing multiple gene expression changes (REG1A, LCN2, NOS2, NNMT, for example).

Gene expression changes in UC, on the other hand, make a strong case for loss of epithelial homeostasis as being central to UC.

IBD Biomarkers

IBD biomarkers include both the UC and CD biomarkers (see Tables 1-9) as well as the following genes and nucleic acids and proteins encoded by the following genes, as well as fragments and variants thereof: CASP10 at 2q33-34, HLA-DMA, TAP1, UBD, PSMB8 at 6p21.3, and PSME2 at 14q11.2. The sequences of these biomarkers are appended to the specification, as well as exemplary primers for amplifying the biomarkers.

Nine genes are elevated in most CD and UC affected profiles and most likely contribute towards separation of IBD from normal controls in the MDS plot. These genes include several chemokine ligands produced by activated monocytes and neutrophils, indicative of an immune/inflammation process and seem to correlate well with the inflammation scoring of the samples by histology (e.g., Group 3)

Certain overlaps evident between the CD and the UC over expressed gene signatures (Table 2. lower panel), involve immune response, antigen presentation (IGHG4, GIP3, LCN2), complement function (C4BPB, DAF), antimicrobial (DEFA6) and general inflammatory response (NOS2A, S100A9, REG1A, PAP).

Further biomarkers for IBD include the proteins or their encoding nucleic acids for the following pathways or cellular processes: apoptosis-regulation (CASP10, LILRB, 1 GNGT1 (7q21.3)), antigen-presenting genes (PSME2), immunoproteasome for generating MHC class I binding antigenic peptides (IBD3, HLA-DMA, TAP1, UBD and PSMB8), and Wnt-signaling (PRKACB (1p36.1, IBD7)).

Corresponding proteins or fragments of proteins for these biomarkers may be represented as intensity peaks in SELDI (surface enhanced laser desorption/ionization) protein chip/mass spectra with molecular masses centered around the values. As discussed above, Markers 1-97, 99-211, 213-264, 266-401 also may be characterized based on affinity for an adsorbent, particularly binding to a cation-exchange or hydrophobic surface under the conditions specified in the Examples, which follow.

The above-identified biomarkers, are examples of biomarkers, as determined by identity, identified by the methods of the invention and serve merely as an illustrative example and are not meant to limit the invention in any way.

A major advantage of identification of these markers is their high specificity and ability to differentiate between different inflammatory bowel disease states (e.g., between UC and CD).

More specifically, the present invention is based upon the discovery of protein markers that are differentially present in samples of human inflammatory bowel disease subjects and control subjects, and the application of this discovery in methods and kits for aiding a human inflammatory bowel disease diagnosis. Some of these protein markers are found at an elevated level and/or more frequently in samples from human inflammatory bowel disease subjects compared to a control (e.g., subjects with diseases other than inflammatory bowel disease). Accordingly, the amount of one or more markers found in a test sample compared to a control, or the mere detection of one or more markers in the test sample provides useful information regarding probability of whether a subject being tested has inflammatory bowel disease or not, and/or whether a subject being tested has a particular inflammatory bowel disease subtype or not.

The protein of the present invention have a number of other uses. For example, the markers can be used to screen for compounds that modulate the expression of the markers in vitro or in vivo, which compounds in turn may be useful in treating or preventing human inflammatory bowel disease in subjects. In another example, markers can be used to monitor responses to certain treatments of human inflammatory bowel disease. In yet another example, the markers can be used in heredity studies. For instance, certain markers may be genetically linked. This can be determined by, e.g., analyzing samples from a population of human inflammatory bowel disease subjects whose families have a history of inflammatory bowel disease. The results can then be compared with data obtained from, e.g., inflammatory bowel disease subjects whose families do not have a history of inflammatory bowel disease. The markers that are genetically linked may be used as a tool to determine if a subject whose family has a history of inflammatory bowel disease is pre-disposed to having inflammatory bowel disease.

In another aspect, the invention provides methods for detecting markers which are differentially present in the samples of an inflammatory bowel disease patient and a control (e.g., subjects in non-inflammatory bowel disease subjects). The markers can be detected in a number of biological samples. The sample is preferably a biological biopsy sample.

Any suitable methods can be used to detect one or more of the markers described herein. These methods include, without limitation, mass spectrometry (e.g., laser desorption/ionization mass spectrometry), fluorescence (e.g. sandwich immunoassay), surface plasmon resonance, ellipsometry and atomic force microscopy. Methods may further include, by one or more of microarrays, PCR methods, electrospray ionization mass spectrometry (ESI-MS), ESI-MS/MS, ESI-MS/(MS)ⁿ, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)ⁿ, atmospheric pressure photoionization mass spectrometry (APPI-MS), APPI-MS/MS, and APPI-(MS).sub.n, quadrupole mass spectrometry, fourier transform mass spectrometry (FTMS), and ion trap mass spectrometry, where n is an integer greater than zero.

The following example is illustrative of the methods used to identify biomarkers for detection of inflammatory bowel disease. It is not meant to limit or construe the invention in any way. A sample, such as for example, serum from a subject or patient, is immobilized on a biochip. Preferably, the biochip comprises a functionalized, cross-linked polymer in the form of a hydrogel physically attached to the surface of the biochip or covalently attached through a silane to the surface of the biochip. However, any biochip which can bind samples from subjects can be used. The surfaces of the biochips are comprised of, for example, hydrophilic adsorbent to capture hydrophilic proteins (e.g. silicon oxide); carboimidizole functional groups that can react with groups on proteins for covalent binding; epoxide functional groups for covalent binding with proteins (e.g. antibodies, receptors, lectins, heparin, Protein A, biotin/streptavidin and the like); anionic exchange groups; cation exchange groups; metal chelators and the like.

Preferably, samples are pre-fractionated prior to immobilization as discussed below. Analytes or samples captured on the surface of a biochip can be detected by any method known in the art. This includes, for example, mass spectrometry, fluorescence, surface plasmon resonance, ellipsometry and atomic force microscopy. Mass spectrometry, and particularly SELDI mass spectrometry, is a particularly useful method for detection of the biomarkers of this invention. Other methods include, chemical extraction partitioning, ion exchange chromatography, reverse phase liquid chromatography, isoelectric focusing, one-dimensional polyacrylamide gel electrophoresis (PAGE), two-dimensional polyacrylamide gel electrophoresis (2D-PAGE), thin-layer chromatography, gas chromatography, liquid chromatography, and any combination thereof.

Immobilized samples or analytes are preferably subjected to laser ionization and the intensity of signal for mass/charge ratio is detected. The data obtained from the mass/charge ratio signal is transformed into data which is read by any type of computer. An algorithm is executed by the computer user that classifies the data according to user input parameters for detecting signals that represent biomarkers present in, for example, inflammatory bowel disease subjects and are lacking in non-inflammatory bowel disease subject controls. The biomarkers are most preferably identified by their molecular weights.

### Test Samples

Subject Types

Samples are collected from subjects to establish inflammatory bowel disease status. The subjects may be subjects who have been determined to have a high risk of inflammatory bowel disease based on their family history, a previous treatment, subjects with physical symptoms known to be associated with inflammatory bowel disease, subjects identified through screening assays (e.g., sigmoidoscopy) or rectal digital exam or rigid or flexible colonoscopy or CT scan or other x-ray techniques. Other subjects include subjects who have inflammatory bowel disease and the test is being used to determine the effectiveness of therapy or treatment they are receiving. Also, subjects could include healthy people who are having a test as part of a routine examination, or to establish baseline levels of the biomarkers. Samples may be collected from subjects who had been diagnosed with inflammatory bowel disease and received treatment to eliminate the inflammatory bowel disease, or perhaps are in remission.

Types of Sample and Preparation of the Sample

The markers can be measured in different types of biological samples. The sample is preferably a biological tissue or fluid sample. Examples of biological tissue sample is a colon or intestinal biopsy sample, from for example a endoscopic examination. Examples of a biological fluid sample useful in this invention include blood, blood serum, plasma, vaginal secretions, urine, tears, saliva, urine, tissue, cells, organs, seminal fluids, bone marrow, cerebrospinal fluid, etc. Because the markers are found in intestinal and/or colon tissue, these are preferred sample sources for embodiments of the invention.

Nucleic acids may be obtained from the samples in many ways known to one of skill in the art. For example, extraction methods, including for example, solvent extraction, affinity purification and centrifugation. Selective precipitation can also purify nucleic acids. Chromatography methods may also be utilized including, gel filtration, ion exchange, selective adsorption, or affinity binding. The nucleic acids may be, for example, RNA, DNA or may be synthesized into cDNA. The nucleic acids may be detected using microarray techniques that are well known in the art, for example, Affymetrix arrays followed by multi-dimensional scaling techniques. See R. Ekins and F. W. Chu, Microarrays: their origins and applications. Trends in Biotechnology, 1999, 17, 217-218; D. D. Shoemaker, et al., Experimental annotation of the human genome using microarray technology, Nature Volume 409 Number 6822 Page 922-927 (2001) and U.S. Pat. No. 5,750,015.

The markers can be resolved in a sample by using a variety of techniques, e.g., nucleic acid chips, PCR, real time PCR, reverse transcriptase PCR, real time reverse transcriptase PCR, in situ PCR, chromatographic separation coupled with mass spectrometry, protein capture using immobilized antibodies or by traditional immunoassays.

Biomarker expression may also be by PCR methods, including for example, real time PCR. See for example, U.S. Pat. Nos. 5,723,591; 5,801,155 and 6,084,102 and Higuchi, 1992 and 1993. PCR assays may be done, for example, in a multi-well plate formats or in chips, such as the BioTrove OpenArray™ Chips (BioTrove, Woburn, Mass.).

If desired, the sample can be prepared to enhance detectability of the markers. For example, to increase the detectability of markers, a blood serum sample from the subject can be preferably fractionated by, e.g., Cibacron blue agarose chromatography and single stranded DNA affinity chromatography, anion exchange chromatography, affinity chromatography (e.g., with antibodies) and the like. The method of fractionation depends on the type of detection method used. Any method that enriches for the protein of interest can be used. Typically, preparation involves fractionation of the sample and collection of fractions determined to contain the biomarkers. Methods of pre-fractionation include, for example, size exclusion chromatography, ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance proteins, such as albumin, from blood before analysis.

In one embodiment, a sample can be pre-fractionated according to size of proteins in a sample using size exclusion chromatography. For a biological sample wherein the amount of sample available is small, preferably a size selection spin column is used. For example, a K30 spin column (available from Princeton Separation, Ciphergen Biosystems, Inc., etc.) can be used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by gas phase ion spectrometry for the detection of markers.

In another embodiment, a sample can be pre-fractionated by anion exchange chromatography. Anion exchange chromatography allows pre-fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomolecules in a sample that are more negatively charged from other types of biomolecules. Proteins that are eluted with an eluant having a high pH is likely to be weakly negatively charged, and a fraction that is eluted with an eluant having a low pH is likely to be strongly negatively charged. Thus, in addition to reducing complexity of a sample, anion exchange chromatography separates proteins according to their binding characteristics.

In yet another embodiment, a sample can be pre-fractionated by heparin chromatography. Heparin chromatography allows pre-fractionation of the markers in a sample also on the basis of affinity interaction with heparin and charge characteristics. Heparin, a sulfated mucopolysaccharide, will bind markers with positively charged moieties and a sample can be sequentially eluted with eluants having different pH's or salt concentrations. Markers eluted with an eluant having a low pH are more likely to be weakly positively charged. Markers eluted with an eluant having a high pH are more likely to be strongly positively charged. Thus, heparin chromatography also reduces the complexity of a sample and separates markers according to their binding characteristics.

In yet another embodiment, a sample can be pre-fractionated by removing proteins that are present in a high quantity or that may interfere with the detection of markers in a sample. For example, in a blood serum sample, serum albumin is present in a high quantity and may obscure the analysis of markers. Thus, a blood serum sample can be pre-fractionated by removing serum albumin. Serum albumin can be removed using a substrate that comprises adsorbents that specifically bind serum albumin. For example, a column which comprises, e.g., Cibacron blue agarose (which has a high affinity for serum albumin) or anti-serum albumin antibodies can be used.

In yet another embodiment, a sample can be pre-fractionated by isolating proteins that have a specific characteristic, e.g. are glycosylated. For example, a blood serum sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

Many types of affinity adsorbents exist which are suitable for pre-fractionating blood serum samples. An example of one other type of affinity chromatography available to pre-fractionate a sample is a single stranded DNA spin column. These columns bind proteins which are basic or positively charged. Bound proteins are then eluted from the column using eluants containing denaturants or high pH.

Thus there are many ways to reduce the complexity of a sample based on the binding properties of the proteins in the sample, or the characteristics of the proteins in the sample.

In yet another embodiment, a sample can be fractionated using a sequential extraction protocol. In sequential extraction, a sample is exposed to a series of adsorbents to extract different types of biomolecules from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomolecules. In this embodiment, the sample is applied to a first adsorbent on the probe, which is subsequently washed with an eluant. Markers that do not bind to the first adsorbent is removed with an eluant. The markers that are in the fraction can be applied to a second adsorbent on the probe, and so forth. The advantage of performing sequential extraction on a gas phase ion spectrometer probe is that markers that bind to various adsorbents at every stage of the sequential extraction protocol can be analyzed directly using a gas phase ion spectrometer.

In yet another embodiment, biomolecules in a sample can be separated by high-resolution electrophoresis, e.g., one or two-dimensional gel electrophoresis. A fraction containing a marker can be isolated and further analyzed by gas phase ion spectrometry. Preferably, two-dimensional gel electrophoresis is used to generate two-dimensional array of spots of biomolecules, including one or more markers. See, e.g., Jungblut and Thiede, Mass Spectr. Rev. 16:145-162 (1997).

The two-dimensional gel electrophoresis can be performed using methods known in the art. See, e.g., Deutscher ed., Methods In Enzymology vol. 182. Typically, biomolecules in a sample are separated by, e.g., isoelectric focusing, during which biomolecules in a sample are separated in a pH gradient until they reach a spot where their net charge is zero (i.e., isoelectric point). This first separation step results in one-dimensional array of biomolecules. The biomolecules in one-dimensional array is further separated using a technique generally distinct from that used in the first separation step. For example, in the second dimension, biomolecules separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of biomolecules. Typically, two-dimensional gel electrophoresis can separate chemically different biomolecules in the molecular mass range from 1000-200,000 Da within complex mixtures.

Biomolecules in the two-dimensional array can be detected using any suitable methods known in the art. For example, biomolecules in a gel can be labeled or stained (e.g., Coomassie Blue or silver staining). If gel electrophoresis generates spots that correspond to the molecular weight of one or more markers of the invention, the spot can be is further analyzed by gas phase ion spectrometry. For example, spots can be excised from the gel and analyzed by gas phase ion spectrometry. Alternatively, the gel containing biomolecules can be transferred to an inert membrane by applying an electric field. Then a spot on the membrane that approximately corresponds to the molecular weight of a marker can be analyzed by gas phase ion spectrometry. In gas phase ion spectrometry, the spots can be analyzed using any suitable techniques, such as MALDI or SELDI (e.g., using ProteinChip® array) as described in detail below.

Prior to gas phase ion spectrometry analysis, it may be desirable to cleave biomolecules in the spot into smaller fragments using cleaving reagents, such as proteases (e.g., trypsin). The digestion of biomolecules into small fragments provides a mass fingerprint of the biomolecules in the spot, which can be used to determine the identity of markers if desired.

In yet another embodiment, high performance liquid chromatography (HPLC) can-be used to separate a mixture of biomolecules in a sample based on their different physical properties, such as polarity, charge and size. HPLC instruments typically consist of a reservoir of mobile phase, a pump, an injector, a separation column, and a detector. Biomolecules in a sample are separated by injecting an aliquot of the sample onto the column. Different biomolecules in the mixture pass through the column at different rates due to differences in their partitioning behavior between the mobile liquid phase and the stationary phase. A fraction that corresponds to the molecular weight and/or physical properties of one or more markers can be collected. The fraction can then be analyzed by gas phase ion spectrometry to detect markers. For example, the spots can be analyzed using either MALDI or SELDI (e.g., using ProteinChip® array) as described in detail below.

Optionally, a marker can be modified before analysis to improve its resolution or to determine its identity. For example, the markers may be subject to proteolytic digestion before analysis. Any protease can be used. Proteases, such as trypsin, that are likely to cleave the markers into a discrete number of fragments are particularly useful. The fragments that result from digestion function as a fingerprint for the markers, thereby enabling their detection indirectly. This is particularly useful where there are markers with similar molecular masses that might be confused for the marker in question. Also, proteolytic fragmentation is useful for high molecular weight markers because smaller markers are more easily resolved by mass spectrometry. In another example, biomolecules can be modified to improve detection resolution. For instance, neuraminidase can be used to remove terminal sialic acid residues from glycoproteins to improve binding to an anionic adsorbent (e.g., cationic exchange ProteinChip® arrays) and to improve detection resolution. In another example, the markers can be modified by the attachment of a tag of particular molecular weight that specifically bind to molecular markers, further distinguishing them. Optionally, after detecting such modified markers, the identity of the markers can be further determined by matching the physical and chemical characteristics of the modified markers in a protein database (e.g., SwissProt).

Detection and Measurement of Markers

Once captured on a substrate, e.g., biochip or antibody, any suitable method can be used to measure a marker or markers in a sample. For example, markers can be detected and/or measured by a variety of detection methods including for example, gas phase ion spectrometry methods, optical methods, electrochemical methods, atomic force microscopy, radio frequency methods, surface plasmon resonance, ellipsometry and atomic force microscopy.

SELDI

One preferred method of detection and/or measurement of the biomarkers uses mass spectrometry, and in particular, “Surface-enhanced laser desorption/ionization” or “SELDI”. SELDI refers to a method of desorption/ionization gas phase ion spectrometry (e.g., mass spectrometry) in which the analyte is captured on the surface of a SELDI probe that engages the probe interface. In “SELDI MS,” the gas phase ion spectrometer is a mass spectrometer. SELDI technology is described in more detail above and as follows.

Preferably, a laser desorption time-of-flight mass spectrometer is used in embodiments of the invention. In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Markers on the substrate surface can be desorbed and ionized using gas phase ion spectrometry. Any suitable gas phase ion spectrometers can be used as long as it allows markers on the substrate to be resolved. Preferably, gas phase ion spectrometers allow quantitation of markers.

In one embodiment, a gas phase ion spectrometer is a mass spectrometer. In a typical mass spectrometer, a substrate or a probe comprising markers on its surface is introduced into an inlet system of the mass spectrometer. The markers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of markers or other substances will typically involve detection of signal intensity. This, in turn, can reflect the quantity and character of markers bound to the substrate. Any of the components of a mass spectrometer (e.g., a desorption source, a mass analyzer, a detector, etc.) can be combined with other suitable components described herein or others known in the art in embodiments of the invention.

Preferably, a laser desorption time-of-flight mass spectrometer is used in embodiments of the invention. In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

In another embodiment, an ion mobility spectrometer can be used to detect markers. The principle of ion mobility spectrometry is based on different mobility of ions. Specifically, ions of a sample produced by ionization move at different rates, due to their difference in, e.g., mass, charge, or shape, through a tube under the influence of an electric field. The ions (typically in the form of a current) are registered at the detector which can then be used to identify a marker or other substances in a sample. One advantage of ion mobility spectrometry is that it can operate at atmospheric pressure.

In yet another embodiment, a total ion current measuring device can be used to detect and characterize markers. This device can be used when the substrate has a only a single type of marker. When a single type of marker is on the substrate, the total current generated from the ionized marker reflects the quantity and other characteristics of the marker. The total ion current produced by the marker can then be compared to a control (e.g., a total ion current of a known compound). The quantity or other characteristics of the marker can then be determined.

Immunoassay

In another embodiment, an immunoassay can be used to detect and analyze markers in a sample. This method comprises: (a) providing an antibody that specifically binds to a marker; (b) contacting a sample with the antibody; and (c) detecting the presence of a complex of the antibody bound to the marker in the sample.

An immunoassay is an assay that uses an antibody to specifically bind an antigen (e.g., a marker). The immunoassay is characterized by the use of specific binding properties of a particular antibody to isolate, target, and/or quantify the antigen. The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a marker from specific species such as rat, mouse, or human can be selected to obtain only those polyclonal antibodies that are specifically immunoreactive with that marker and not with other proteins, except for polymorphic variants and alleles of the marker. This selection may be achieved by subtracting out antibodies that cross-react with the marker molecules from other species.

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Typically a specific or selective reaction will be at least twice background signal or noise and more typically more than 10 to 100 times background.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or ProteinChip® array described above. The sample is preferably a biological fluid sample taken from a subject. Examples of biological fluid samples include blood, serum, plasma, nipple aspirate, urine, tears, saliva etc. In a preferred embodiment, the biological fluid comprises blood serum. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Methods for measuring the amount of, or presence of, antibody-marker complex include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Electrochemical methods include voltametry and amperometry methods. Radio frequency methods include multipolar resonance spectroscopy. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

Immunoassays can be used to determine presence or absence of a marker in a sample as well as the quantity of a marker in a sample. The amount of an antibody-marker complex can be determined by comparing to a standard. A standard can be, e.g., a known compound or another protein known to be present in a sample. As noted above, the test amount of marker need not be measured in absolute units, as long as the unit of measurement can be compared to a control.

The methods for detecting these markers in a sample have many applications. For example, one or more markers can be measured to aid humaninflammatory bowel disease diagnosis or prognosis. In another example, the methods for detection of the markers can be used to monitor responses in a subject to inflammatory bowel disease treatment. In another example, the methods for detecting markers can be used to assay for and to identify compounds that modulate expression of these markers in vivo or in vitro. In a preferred example, the biomarkers are used to differentiate between the different stages of tumor progression, thus aiding in determining appropriate treatment and extent of metastasis of the tumor.

Use of Modified Forms of a Biomarker

It has been found that proteins frequently exist in a sample in a plurality of different forms characterized by a detectably different mass. These forms can result from either, or both, of pre- and post-translational modification. Pre-translational modified forms include allelic variants, slice variants and RNA editing forms. Post-translationally modified forms include forms resulting from proteolytic cleavage (e.g., fragments of a parent protein), glycosylation, phosphorylation, lipidation, oxidation, methylation, cystinylation, sulphonation and acetylation. The collection of proteins including a specific protein and all modified forms of it is referred to herein as a “protein cluster.” The collection of all modified forms of a specific protein, excluding the specific protein, itself, is referred to herein as a “modified protein cluster.” Modified forms of any biomarker of this invention (including any of Markers I through XIII) also may be used, themselves, as biomarkers. In certain cases the modified forms may exhibit better discriminatory power in diagnosis than the specific forms set forth herein.

Modified forms of a biomarker including any of Markers 1-97, 99-211, 213-264, 266-401 can be initially detected by any methodology that can detect and distinguish the modified from the biomarker. A preferred method for initial detection involves first capturing the biomarker and modified forms of it, e.g., with biospecific capture reagents, and then detecting the captured proteins by mass spectrometry. More specifically, the proteins are captured using biospecific capture reagents, such as antibodies, aptamers or Affibodies that recognize the biomarker and modified forms of it. This method also will also result in the capture of protein interactors that are bound to the proteins or that are otherwise recognized by antibodies and that, themselves, can be biomarkers. Preferably, the biospecific capture reagents are bound to a solid phase. Then, the captured proteins can be detected by SELDI mass spectrometry or by eluting the proteins from the capture reagent and detecting the eluted proteins by traditional MALDI or by SELDI. The use of mass spectrometry is especially attractive because it can distinguish and quantify modified forms of a protein based on mass and without the need for labeling.

Preferably, the biospecific capture reagent is bound to a solid phase, such as a bead, a plate, a membrane or a chip. Methods of coupling biomolecules, such as antibodies, to a solid phase are well known in the art. They can employ, for example, bifunctional linking agents, or the solid phase can be derivatized with a reactive group, such as an epoxide or an imidizole, that will bind the molecule on contact. Biospecific capture reagents against different target proteins can be mixed in the same place, or they can be attached to solid phases in different physical or addressable locations. For example, one can load multiple columns with derivatized beads, each column able to capture a single protein cluster. Alternatively, one can pack a single column with different beads derivatized with capture reagents against a variety of protein clusters, thereby capturing all the analytes in a single place. Accordingly, antibody-derivatized bead-based technologies, such as xMAP technology of Luminex (Austin, Tex.) can be used to detect the protein clusters. However, the biospecific capture reagents must be specifically directed toward the members of a cluster in order to differentiate them.

In yet another embodiment, the surfaces of biochips can be derivatized with the capture reagents directed against protein clusters either in the same location or in physically different addressable locations. One advantage of capturing different clusters in different addressable locations is that the analysis becomes simpler.

After identification of modified forms of a protein and correlation with the clinical parameter of interest, the modified form can be used as a biomarker in any of the methods of this invention. At this point, detection of the modified from can be accomplished by any specific detection methodology including affinity capture followed by mass spectrometry, or traditional immunoassay directed specifically the modified form. Immunoassay requires biospecific capture reagents, such as antibodies, to capture the analytes. Furthermore, if the assay must be designed to specifically distinguish protein and modified forms of protein. This can be done, for example, by employing a sandwich assay in which one antibody captures more than one form and second, distinctly labeled antibodies, specifically bind, and provide distinct detection of, the various forms. Antibodies can be produced by immunizing animals with the biomolecules. This invention contemplates traditional immunoassays including, for example, sandwich immunoassays including ELISA or fluorescence-based immunoassays, as well as other enzyme immunoassays.

Data Analysis

The methods for detecting these markers in a sample have many applications. For example, one or more markers can be measured to aid human inflammatory bowel disease diagnosis or prognosis. In another example, the methods for detection of the markers can be used to monitor responses in a subject to inflammatory bowel disease treatment. In another example, the methods for detecting markers can be used to assay for and to identify compounds that modulate expression of these markers in vivo or in vitro.

Differentiation of non-inflammatory bowel disease and inflammatory bowel disease status may be by the detection of one or more of the Markers listed in Tables 1-3 or the Markers described as proteins or pathways for IBD, UC, or CD. For example, an exemplary marker that may independently discriminate between colorectal and non-colorectal status is Markers 1-75. Combinations of markers are also useful in the methods of the invention for the discrimination of on-inflammatory bowel disease and inflammatory bowel disease status, for example, Markers may also be used to discriminate or distinguish or diagnose between UC and CD and between unaffected and affected tissue of a UC and/or CD subject.

Markers may be detected, determined, monitored in a sample by molecular biological methods, including, arrays (nucleic acid, protein), PCR methods (real-time, reverse transcriptase, PCR).

Detection of markers can be analyzed using any suitable means, including arrays. Nucleic acid arrays may be analyzed using software, for example, Applied Maths, Belgium. GenExplore™: 2-way cluster analysis, principal component analysis, discriminant analysis, self-organizing maps; BioDiscovery, Inc., Los Angeles, Calif. (ImaGene™, special image processing and data extraction software, powered by MatLab®; GeneSight: hierarchical clustering, artificial neural network (SOM?), principal component analysis, time series; AutoGene™; CloneTracker™); GeneData AG (Basel, Switzerland); Molecular Pattern Recognition web site at MIT's Whitehead Genome Center; Rosetta Inpharmatics, Kirkland, Wash. Resolver™ Expression Data Analysis System; Scanalytics, Inc., Fairfax, Va. Its MicroArray Suite enables researchers to acquire, visualize, process, and analyze gene expression microarray data; TIGR (The Institute for Genome Research) offers software tools (free for academic institutions) for array analysis. For example, see also Eisen M B, Brown PO., Methods Enzymol. 1999; 303:179-205.

Detection of markers can be analyzed using any suitable means. In one embodiment, data generated, for example, by desorption is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of markers detected, including the strength of the signal generated by each marker.

Data analysis can include the steps of determining signal strength (e.g., height of peaks) of a marker detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other biomolecules can be displayed in the form of relative intensities in the scale desired (e.g., 100). Alternatively, a standard (e.g., a serum protein) may be admitted with the sample so that a peak from the standard can be used as a reference to calculate relative intensities of the signals observed for each marker or other markers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique markers and markers which are up- or down-regulated between samples. Marker profiles (spectra) from any two samples may be compared visually. In yet another format, Spotfire Scatter Plot can be used, wherein markers that are detected are plotted as a dot in a plot, wherein one axis of the plot represents the apparent molecular of the markers detected and another axis represents the signal intensity of markers detected. For each sample, markers that are detected and the amount of markers present in the sample can be saved in a computer readable medium. This data can then be compared to a control (e.g., a profile or quantity of markers detected in control, e.g., men in whom human inflammatory bowel disease is undetectable).

When the sample is measured and data is generated, e.g., by mass spectrometry, the data is then analyzed by a computer software program. Generally, the software can comprise code that converts signal from the mass spectrometer into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal” and human IBD and determines the closeness of fit between the two signals. The software also can include code indicating which the test sample is closest to, thereby providing a probable diagnosis.

In preferred methods of the present invention, multiple biomarkers are measured. The use of multiple biomarkers increases the predictive value of the test and provides greater utility in diagnosis, toxicology, patient stratification and patient monitoring. The process called “Pattern recognition” detects the patterns formed by multiple biomarkers greatly improves the sensitivity and specificity of clinical proteomics for predictive medicine. Subtle variations in data from clinical samples, e.g., obtained using SELDI, indicate that certain patterns of protein expression can predict phenotypes such as the presence or absence of a certain disease, a particular stage of IBD-progression, or a positive or adverse response to drug treatments.

Data generation in mass spectrometry begins with the detection of ions by an ion detector as described above. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip software, data processing typically includes TOF-to-M/Z transformation, baseline subtraction, high frequency noise filtering.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

The spectra that are generated in embodiments of the invention can be classified using a pattern recognition process that uses a classification model. In some embodiments, data derived from the spectra (e.g., mass spectra or time-of-flight spectra) that are generated using samples such as “known samples” can then be used to “train” a classification model. A “known sample” is a sample that is pre-classified (e.g., inflammatory bowel disease or not inflammatory bowel disease). Data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased vs. non diseased).

The training data set that is used to form the classification model may comprise raw data or pre-processed data. In some embodiments, raw data can be obtained directly from time-of-flight spectra or mass spectra, and then may be optionally “pre-processed” in any suitable manner. For example, signals above a predetermined signal-to-noise ratio can be selected so that a subset of peaks in a spectrum is selected, rather than selecting all peaks in a spectrum. In another example, a predetermined number of peak “clusters” at a common value (e.g., a particular time-of-flight value or mass-to-charge ratio value) can be used to select peaks. Illustratively, if a peak at a given mass-to-charge ratio is in less than 50% of the mass spectra in a group of mass spectra, then the peak at that mass-to-charge ratio can be omitted from the training data set. Pre-processing steps such as these can be used to reduce the amount of data that is used to train the classification model.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, which is herein incorporated by reference in its entirety.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. 2002 0138208 A1 (Paulse et al., “Method for analyzing mass spectra,” Sep. 26, 2002.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described in, for example, WO 01/31580 (Barnhill et al., “Methods and devices for identifying patterns in biological systems and methods of use thereof,” May 3, 2001); U.S. 2002/0193950 A1 (Gavin et al., “Method or analyzing mass spectra,” Dec. 19, 2002); U.S. 2003/0004402 A1 (Hitt et al., “Process for discriminating between biological states based on hidden patterns from biological data,” Jan. 2, 2003); and U.S. 2003/0055615 A1 (Zhang and Zhang, “Systems and methods for processing biological expression data” Mar. 20, 2003).

More specifically, to obtain the biomarkers the peak intensity data of samples from subjects, e.g., IBD subjects, and healthy controls are used as a “discovery set.” This data were combined and randomly divided into a training set and a test set to construct and test multivariate predictive models using a non-linear version of Unified Maximum Separability Analysis (“USMA”) classifiers. Details of USMA classifiers are described in U.S. 2003/0055615 A1.

The invention provides methods for aiding a human inflammatory bowel disease diagnosis using one or more markers, for example Markers in the tables and figures which follow, and including one or more Markers 1-97, 99-211, 213-264, 266-401 as specified herein. These markers can be used alone, in combination with other markers in any set, or with entirely different markers in aiding human inflammatory bowel disease diagnosis. The markers are differentially present in samples of a human inflammatory bowel disease patient and a normal subject in whom human inflammatory bowel disease is undetectable. For example, some of the markers are expressed at an elevated level and/or are present at a higher frequency in human inflammatory bowel disease subjects than in normal subjects, while some of the markers are expressed at a decreased level and/or are present at a lower frequency in human inflammatory bowel disease subjects than in normal subjects. Therefore, detection of one or more of these markers in a person would provide useful information regarding the probability that the person may have inflammatory bowel disease.

Differentiation Between Normal and Unaffected Disease Tissue

The invention provides methods for aiding a human inflammatory bowel disease diagnosis using one or more markers, for example Markers in the tables and figures which follow, and including one or more Markers 1-97, 99-211, 213-264, 266-401 as specified herein. These markers can be used alone, in combination with other markers in any set, or with entirely different markers in aiding human inflammatory bowel disease diagnosis. The markers are differentially present in samples of a human inflammatory bowel disease patient and a normal subject in whom human inflammatory bowel disease is undetectable. For example, some of the markers are expressed at an elevated level and/or are present at a higher frequency in human inflammatory bowel disease subjects than in normal subjects, while some of the markers are expressed at a decreased level and/or are present at a lower frequency in human inflammatory bowel disease subjects than in normal subjects. Therefore, detection of one or more of these markers in a person would provide useful information regarding the probability that the person may have inflammatory bowel disease.

In a preferred embodiment, a biological sample is collected from a patient and then either left unfractionated, or fractionated using an anion exchange resin as described above. The biomarkers in the sample are captured using an ProteinChip array. The markers are then detected using SELDI. The results are then entered into a computer system, which contains an algorithm that is designed using the same parameters that were used in the learning algorithm and classification algorithm to originally determine the biomarkers. The algorithm produces a diagnosis based upon the data received relating to each biomarker.

The diagnosis is determined by examining the data produced from the tests with algorithms that are developed using the biomarkers. The algorithms depend on the particulars of the test protocol used to detect the biomarkers. These particulars include, for example, sample preparation, chip type and mass spectrometer parameters. If the test parameters change, the algorithm must change. Similarly, if the algorithm changes, the test protocol must change.

In another embodiment, the sample is collected from the patient. The biomarkers are captured using an antibody ProteinChip array as described above. The markers are detected using a biospecific SELDI test system. The results are then entered into a computer system, which contains an algorithm that is designed using the same parameters that were used in the learning algorithm and classification algorithm to originally determine the biomarkers. The algorithm produces a diagnosis based upon the data received relating to each biomarker.

In yet other preferred embodiments, the markers are captured and tested using non-SELDI formats. In one example, the sample is collected from the patient. The biomarkers are captured on a substrate using other known means, e.g., antibodies to the markers. The markers are detected using methods known in the art, e.g., optical methods and refractive index. Examples of optical methods include detection of fluorescence, e.g., ELISA. Examples of refractive index include surface plasmon resonance. The results for the markers are then subjected to an algorithm, which may or may not require artificial intelligence. The algorithm produces a diagnosis based upon the data received relating to each biomarker.

In any of the above methods, the data from the sample may be fed directly from the detection means into a computer containing the diagnostic algorithm. Alternatively, the data obtained can be fed manually, or via an automated means, into a separate computer that contains the diagnostic algorithm.

Accordingly, embodiments of the invention include methods for aiding a human inflammatory bowel disease diagnosis, wherein the method comprises: (a) detecting at least one marker in a sample, wherein the marker is selected from any of the Markers 1-97, 99-211, 213-264, 266-401; and (b) correlating the detection of the marker or markers with a probable diagnosis of human inflammatory bowel disease. The correlation may take into account the amount of the marker or markers in the sample compared to a control amount of the marker or markers (up or down regulation of the marker or markers) (e.g., in normal subjects in whom human inflammatory bowel disease is undetectable). The correlation may take into account the presence or absence of the markers in a test sample and the frequency of detection of the same markers in a control. The correlation may take into account both of such factors to facilitate determination of whether a subject has a human inflammatory bowel disease or not.

In a preferred embodiment, Markers 1-97, 99-211, 213-264, 266-401 are used to make a correlation with inflammatory bowel disease, wherein the inflammatory bowel disease may be any subtype, e.g., Crohn's disease or ulcerative colitis.

Any suitable samples can be obtained from a subject to detect markers. Preferably, a sample is a colon or intestinal biopsy, e.g., an endoscopic biopsy sample from the subject. If desired, the sample can be prepared as described above to enhance detectability of the markers. For example, to increase the detectability of markers, a sample from the subject can be preferably fractionated by, e.g., Cibacron blue agarose chromatography and single stranded DNA affinity chromatography, anion exchange chromatography and the like. Sample preparations, such as pre-fractionation protocols, are optional and may not be necessary to enhance detectability of markers depending on the methods of detection used. For example, sample preparation may be unnecessary if antibodies that specifically bind markers are used to detect the presence of markers in a sample.

Processes for the purification of a biomarker include fractioning a sample, as described herein, for example, by size-exclusion chromatography and collecting a fraction that includes one or more biomarkers; and/or fractionating a sample comprising the one or more biomarkers by anion exchange chromatography and collecting a fraction that includes one or more biomarkers, wherein the biomarker is selected from one or more of the biomarkers of Tables 1-9.

### IBD Candidate Genes

In one aspect the invention also includes IBD candidate genes. These genes include, for example, apoptosis-regulating CASP10 at 2q33-34, LILRB1 at 19q13.4 (locus IBD6) and antigen-presenting genes PSME2 at 14q11.2 (locus IBD4). With respect to the IBD3 locus at 6p21,³⁵ HLA-DMA, TAP1, UBD and PSMB8 (immunoproteasome for generating MHC class I binding antigenic peptides), at 6p21.3, are particularly intriguing. GNGT1 (7q21.3) functioning in apoptosis and PRKACB (1p36.1, IBD7), involved in Wnt-signaling from the UC signature are also good candidates. The sequences of these genes are appended to the end of this specification, as well as exemplary primers for detecting or amplifying the makers.

### Diagnosis of Subject and Determination of Inflammatory Bowel Disease Status

Any biomarker, individually, is useful in aiding in the determination of inflammatory bowel disease status. First, the selected biomarker is measured in a subject sample using the methods described herein, e.g., capture on a SELDI biochip followed by detection by mass spectrometry. Then, the measurement is compared with a diagnostic amount or control that distinguishes a inflammatory bowel disease status from a non-inflammatory bowel disease status. The diagnostic amount will reflect the information herein that a particular biomarker is up-regulated or down-regulated in a inflammatory bowel disease status compared with a non-inflammatory bowel disease status. As is well understood in the art, the particular diagnostic amount used can be adjusted to increase sensitivity or specificity of the diagnostic assay depending on the preference of the diagnostician. The test amount as compared with the diagnostic amount thus indicates inflammatory bowel disease status.

While individual biomarkers are useful diagnostic markers, it has been found that a combination of biomarkers provides greater predictive value than single markers alone. Specifically, the detection of a plurality of markers in a sample increases the percentage of true positive and true negative diagnoses and would decrease the percentage of false positive or false negative diagnoses. Thus, preferred methods of the present invention comprise the measurement of more than one biomarker.

The detection of the marker or markers is then correlated with a probable diagnosis of inflammatory bowel disease. In some embodiments, the detection of the mere presence or absence of a marker, without quantifying the amount of marker, is useful and can be correlated with a probable diagnosis of inflammatory bowel disease. For example, biomarkers 1-97, 99-211, 213-264, 266-401 can be more frequently detected in human inflammatory bowel disease subjects than in normal subjects. A mere detection of one or more of these markers in a subject being tested indicates that the subject has a higher probability of having inflammatory bowel disease. In another embodiment, biomarkers 61-75 can be less frequently detected in human UC disease subjects than in normal subjects, and/or in subjects who have CD. The mere detection of one or more of these markers in a subject being tested indicates that the subject has a lower probability of having inflammatory bowel disease.

In other embodiments, the measurement of markers can involve quantifying the markers to correlate the detection of markers with a probable diagnosis of inflammatory bowel disease. Thus, if the amount of the markers detected in a subject being tested is different compared to a control amount (i.e., higher or lower than the control, depending on the marker), then the subject being tested has a higher probability of having inflammatory bowel disease.

The correlation may take into account the amount of the marker or markers in the sample compared to a control amount of the marker or markers (up or down regulation of the marker or markers) (e.g., in normal subjects or in non-inflammatory bowel disease subjects such as where inflammatory bowel disease is undetectable). A control can be, e.g., the average or median amount of marker present in comparable samples of normal subjects in normal subjects or in non-inflammatory bowel disease subjects such as where inflammatory bowel disease is undetectable. The control amount is measured under the same or substantially similar experimental conditions as in measuring the test amount. The correlation may take into account the presence or absence of the markers in a test sample and the frequency of detection of the same markers in a control. The correlation may take into account both of such factors to facilitate determination of inflammatory bowel disease status.

In certain embodiments of the methods of qualifying inflammatory bowel disease status, the methods further comprise managing subject treatment based on the status. As before the, management of the subject describes the actions of the physician or clinician subsequent to determining inflammatory bowel disease status. For example, if the result of the methods of the present invention is inconclusive or there is reason that confirmation of status is necessary, the physician may order more tests (e.g., colonoscopy and imaging techniques). Alternatively, if the status indicates that treatment is appropriate, the physician may schedule the patient for treatment. In other instances, the patient may receive therapeutic treatments, either in lieu of, or in addition to, surgery. No further action may be warranted. Furthermore, if the results show that treatment has been successful, a maintenance therapy or no further management may be necessary.

Therapeutic agents may include, one or more of sulfa drugs, corticosteriods (prednisone), 5-aminosalicylates (Asacol, Pentasa, Rowasa, or 5-ASA), immunosuppressives (azathioprine, Imuran, Cyclosporine, 6-MP, Purinethol and Methotrexate), anti-TNF (Remicade), anticholinergics, dicyclomine (Bentyl), belladonna/phenobarbital (Donnatal, Antispas, bBarbidonna, donnapine, hyosophen, Spasmolin), hyoscyamine (Levsin, Anaspaz), chlordiazepoxide/clidinium (Librax), anti-diarrheals, diphenoxylate/atropine (Lomotil), alosetron hydrochloride (Lotronex), tegaserod (Zelnorm, Zelmac), rifaximin (Xifaxin), sulfasalazine (Azulfadine), mesalamine (Asacol, Pentasa, Rowasa), osalazine (Dipentum), (Colazal), corticosteroids (prednisone), balsalazide disodium (Colazal®), cyclosporine, methotrexate, infliximab (Remicade), rifaximin, and budesonide (Entocort EC)

The invention also provides for such methods where the biomarkers (or specific combination of biomarkers) are measured again after subject management. In these cases, the methods are used to monitor the status of the inflammatory bowel disease, e.g., response to inflammatory bowel disease treatment, remission of the disease or progression of the disease. Because of the ease of use of the methods and the lack of invasiveness of the methods, the methods can be repeated after each treatment the patient receives. This allows the physician to follow the effectiveness of the course of treatment. If the results show that the treatment is not effective, the course of treatment can be altered accordingly. This enables the physician to be flexible in the treatment options.

In another example, the methods for detecting markers can be used to assay for and to identify compounds that modulate expression of these markers in vivo or in vitro.

The methods of the present invention have other applications as well. For example, the markers can be used to screen for compounds that modulate the expression of the markers in vitro or in vivo, which compounds in turn may be useful in treating or preventing inflammatory bowel disease in subjects. In another example, the markers can be used to monitor the response to treatments for inflammatory bowel disease. In yet another example, the markers can be used in heredity studies to determine if the subject is at risk for developing inflammatory bowel disease. For instance, certain markers may be genetically linked. This can be determined by, e.g., analyzing samples from a population of inflammatory bowel disease subjects whose families have a history of inflammatory bowel disease. The results can then be compared with data obtained from, e.g., inflammatory bowel disease subjects whose families do not have a history of inflammatory bowel disease. The markers that are genetically linked may be used as a tool to determine if a subject whose family has a history of inflammatory bowel disease is pre-disposed to having inflammatory bowel disease.

In a preferred embodiment of the invention, a diagnosis based on the presence or absence in a test subject of any the biomarkers of this invention is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

Methods of the invention for determining the inflammatory bowel disease status of a subject, include for example, obtaining a biomarker profile from a sample taken from the subject; and comparing the subject's biomarker profile to a reference biomarker profile obtained from a reference population, wherein the comparison is capable of classifying the subject as belonging to or not belonging to the reference population; wherein the subject's biomarker profile and the reference biomarker profile comprise one or more markers listed in Tables 1-9.

The method may further comprise repeating the method at least once, wherein the subject's biomarker profile is obtained from a separate sample taken each time the method is repeated.

Samples from the subject may be taken at any time, for example, the samples may be taken 24 hours apart or any other time determined useful.

Such comparisons of the biomarker profiles can determine inflammatory bowel disease status in the subject with an accuracy of at least about 60%, 70%, 80%, 90%, 95%, and approaching 100% as shown in the examples which follow.

The reference biomarker profile can be obtained from a population comprising a single subject, at least two subjects, at least 20 subjects or more. The number of subjects will depend, in part, on the number of available subjects, and the power of the statistical analysis necessary.

A method of treating inflammatory bowel disease comprising administering to a subject suffering from or at risk of developing inflammatory bowel disease a therapeutically effective amount of a compound capable of modulating the expression or activity of one or more of the biomarkers of Tables 1-9.

A method of treating a condition in a subject comprising administering to a subject a therapeutically effective amount of a compound which modulates the expression or activity of one or more of the biomarkers of Tables 1-9.

Compounds useful in methods disclosed herein include, for example, sulfa drugs, corticosteriods (prednisone), 5-aminosalicylates (Asacol, Pentasa, Rowasa, or 5-ASA), immunosuppressives (azathioprine, Imuran, Cyclosporine, 6-MP, Purinethol and Methotrexate), anti-TNF (Remicade), anticholinergics, dicyclomine (Bentyl), belladonna/phenobarbital (Donnatal, Antispas, bBarbidonna, donnapine, hyosophen, Spasmolin), hyoscyamine (Levsin, Anaspaz), chlordiazepoxide/clidinium (Librax), anti-diarrheals, diphenoxylate/atropine (Lomotil), alosetron hydrochloride (Lotronex), tegaserod (Zelnorm, Zelmac), rifaximin (Xifaxin), sulfasalazine (Azulfadine), mesalamine (Asacol, Pentasa, Rowasa), osalazine (Dipentum), (Colazal), corticosteroids (prednisone), balsalazide disodium (Colazal®), cyclosporine, methotrexate, infliximab (Remicade), rifaximin, and budesonide (Entocort EC)

A method of qualifying inflammatory bowel disease status in a subject comprising:

(a) measuring at least one biomarker in a sample from the subject, wherein the biomarker is selected from one or more of the biomarkers of Tables 1-9, and

(b) correlating the measurement with inflammatory bowel disease status.

The method may also comprise the step of measuring the at least one biomarker after subject management.

Optionally, the methods of the invention may further comprise generating data on immobilized subject samples on a biochip, by subjecting the biochip to laser ionization and detecting intensity of signal for mass/charge ratio; and transforming the data into computer readable form; and executing an algorithm that classifies the data according to user input parameters, for detecting signals that represent biomarkers present in inflammatory bowel disease subjects and are lacking in non-inflammatory bowel disease subject controls.

Types of inflammatory bowel disease that may be identified or differentiated from one another according to this method include UC and CD.

Kits

In one aspect, the invention provides kits for the analysis of IBD status. The kits include PCR primers for at least one marker selected from Markers 1-75. In preferred embodiments, the kit includes more than two or three markers selected from Markers 1-75. The kit may further include instructions for use and correlation of the maker with disease status. For example, the presence of any one of Markers 1-31 indicate CD; the presence of any one of Markers 32-48 indicate IBD; the increased presence of any one of Markers 49-60 indicate UC and the decreased presence of any one of Markers 61-75 indicate UC. The kit may also include a DNA array containing the complement of one or more of the Markers selected from 1-75, reagents, and/or enzymes for amplifying or isolating sample DNA. The kits may include reagents for real-time PCR, for example, TaqMan probes and/or primers, and enzymes.

In yet another aspect, the invention provides kits for qualifying inflammatory bowel disease status and/or aiding a diagnosis of human inflammatory bowel disease, wherein the kits can be used to detect the markers of the present invention. For example, the kits can be used to detect any one or more of the markers described herein, which markers are differentially present in samples of inflammatory bowel disease subjects and normal subjects. The kits of the invention have many applications. For example, the kits can be used to differentiate if a subject has inflammatory bowel disease or has a negative diagnosis, thus aiding a human inflammatory bowel disease diagnosis. In another example, the kits can be used to identify compounds that modulate expression of one or more of the markers in in vitro or in vivo animal models for inflammatory bowel disease.

In one embodiment, a kit comprises: (a) a substrate comprising an adsorbent thereon, wherein the adsorbent is suitable for binding a marker, and (b) instructions to detect the marker or markers by contacting a sample with the adsorbent and detecting the marker or markers retained by the adsorbent. In some embodiments, the kit may comprise an eluant (as an alternative or in combination with instructions) or instructions for making an eluant, wherein the combination of the adsorbent and the eluant allows detection of the markers using gas phase ion spectrometry.

Such kits can be prepared from the materials described above, and the previous discussion of these materials (e.g., probe substrates, adsorbents, washing solutions, etc.) is fully applicable to this section and will not be repeated.

In another embodiment, the kit may comprise a first substrate comprising an adsorbent thereon (e.g., a particle functionalized with an adsorbent) and a second substrate onto which the first substrate can be positioned to form a probe, which is removably insertable into a gas phase ion spectrometer. In other embodiments, the kit may comprise a single substrate, which is in the form of a removably insertable probe with adsorbents on the substrate. In yet another embodiment, the kit may further comprise a pre-fractionation spin column (e.g., Cibacron blue agarose column, anti-HSA agarose column, K-30 size exclusion column, Q-anion exchange spin column, single stranded DNA column, lectin column, etc.).

In another embodiment, a kit comprises (a) an antibody that specifically binds to a marker; and (b) a detection reagent. Such kits can be prepared from the materials described above, and the previous discussion regarding the materials (e.g., antibodies, detection reagents, immobilized supports, etc.) is fully applicable to this section and will not be repeated. Optionally, the kit may further comprise pre-fractionation spin columns. In some embodiments, the kit may further comprise instructions for suitable operation parameters in the form of a label or a separate insert.

Optionally, the kit may further comprise a standard or control information so that the test sample can be compared with the control information standard to determine if the test amount of a marker detected in a sample is a diagnostic amount consistent with a diagnosis of inflammatory bowel disease.

Use of Biomarkers for Inflammatory Bowel Disease in Screening Assays

The methods of the present invention have other applications as well. For example, the biomarkers can be used to screen for compounds that modulate the expression of the biomarkers in vitro or in vivo, which compounds in turn may be useful in treating or preventing inflammatory bowel disease in subjects. In another example, the biomarkers can be used to monitor the response to treatments for inflammatory bowel disease. In yet another example, the biomarkers can be used in heredity studies to determine if the subject is at risk for developing inflammatory bowel disease.

Thus, for example, the kits of this invention could include a solid substrate having a hydrophobic function, such as a protein biochip (e.g., a Ciphergen ProteinChip array) and a buffer for washing the substrate, as well as instructions providing a protocol to measure the biomarkers of this invention on the chip and to use these measurements to diagnose inflammatory bowel disease.

Method for identifying a candidate compound for treating inflammatory bowel disease may comprise, for example, contacting one or more of the biomarkers of Tables 1-9 with a test compound; and determining whether the test compound interacts with the biomarker, wherein a compound that interacts with the biomarker is identified as a candidate compound for treating inflammatory bowel disease.

Compounds suitable for therapeutic testing may be screened initially by identifying compounds which interact with one or more biomarkers listed in identified herein. By way of example, screening might include recombinantly expressing a biomarker of this invention, purifying the biomarker, and affixing the biomarker to a substrate. Test compounds would then be contacted with the substrate, typically in aqueous conditions, and interactions between the test compound and the biomarker are measured, for example, by measuring elution rates as a function of salt concentration. Certain proteins may recognize and cleave one or more biomarkers of this invention, in which case the proteins may be detected by monitoring the digestion of one or more biomarkers in a standard assay, e.g., by gel electrophoresis of the proteins.

In a related embodiment, the ability of a test compound to inhibit the activity of one or more of the biomarkers of this invention may be measured. One of skill in the art will recognize that the techniques used to measure the activity of a particular biomarker will vary depending on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the activity of a given biomarker may be determined by measuring the rates of catalysis in the presence or absence of the test compounds. The ability of a test compound to interfere with a non-enzymatic (e.g., structural) function or activity of one of the biomarkers of this invention may also be measured. For example, the self-assembly of a multi-protein complex which includes one of the biomarkers of this invention may be monitored by spectroscopy in the presence or absence of a test compound. Alternatively, if the biomarker is a non-enzymatic enhancer of transcription, test compounds which interfere with the ability of the biomarker to enhance transcription may be identified by measuring the levels of biomarker-dependent transcription in vivo or in vitro in the presence and absence of the test compound.

Test compounds capable of modulating the activity of any of the biomarkers of this invention may be administered to subjects who are suffering from or are at risk of developing inflammatory bowel disease. For example, the administration of a test compound which increases the activity of a particular biomarker may decrease the risk of inflammatory bowel disease in a patient if the activity of the particular biomarker in vivo prevents the accumulation of proteins for inflammatory bowel disease. Conversely, the administration of a test compound which decreases the activity of a particular biomarker may decrease the risk of inflammatory bowel disease in a patient if the increased activity of the biomarker is responsible, at least in part, for the onset of inflammatory bowel disease.

At the clinical level, screening a test compound includes obtaining samples from test subjects before and after the subjects have been exposed to a test compound. The levels in the samples of one or more of the biomarkers of this invention may be measured and analyzed to determine whether the levels of the biomarkers change after exposure to a test compound. The samples may be analyzed by mass spectrometry, as described herein, or the samples may be analyzed by any appropriate means known to one of skill in the art. For example, the levels of one or more of the biomarkers of this invention may be measured directly by Western blot using radio- or fluorescently-labeled antibodies which specifically bind to the biomarkers. Alternatively, changes in the levels of mRNA encoding the one or more biomarkers may be measured and correlated with the administration of a given test compound to a subject. In a further embodiment, the changes in the level of expression of one or more of the biomarkers may be measured using in vitro methods and materials. For example, human tissue cultured cells which-express, or are capable of expressing, one or more of the biomarkers of this invention may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with inflammatory bowel disease, test compounds will be screened for their ability to slow or stop the progression of the disease.

Classification Algorithms

A dataset can be analyzed by multiple classification algorithms. Some classification algorithms provide discrete rules for classification; others provide probability estimates of a certain outcome (class). In the latter case, the decision (diagnosis) is made based on the class with the highest probability. For example, consider the three-class problem: healthy, benign, and IBD. Suppose that a classification algorithm (e.g. Nearest neighbor) is constructed and applied to sample A, and the probability of the sample being healthy is 0, benign is 33%, and IBD is 67%. Sample A would be diagnosed as being IBD. This approach, however, does not take into account any “fuzziness” in the diagnosis i.e. that there was a certain probability that the sample was benign. Therefore, the diagnosis would be the same as for sample B, which has a probability of 0 of being healthy or benign and a probability of 1 of being IBD.

### EXAMPLES

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

It should be appreciated that the invention should not be construed to be limited to the examples which are now described; rather, the invention should be construed to include any and all applications provided herein and all equivalent variations within the skill of the ordinary artisan.

### Example 1

### Patients and Controls

Informed consent was obtained from all individuals and diagnosis of patients was based on primary endoscopic, pathologic and radiology reports using standard diagnostic criteria.¹⁶ Consecutive patients undergoing colonoscopy include unrelated CD and UC patients and 4 non-IBD healthy controls (Table 10). Controls were negative for colorectal cancer on screening. All patients received Golytely® colonic preparation.

TABLE 10
Demographics

| | | | Disease | | | | | |
|---|---|---|---|---|---|---|---|---|
| | Age | | | Duration | Endoscopy | | Histology | |
| Sample* | (years) | Sex | location | (years) | Site** | definition | Inflammation# | Fibrosis## |
| CD33un | 24 | M | colonic | 4 | T. colon | unaffected | − | − |
| CD33aff | | | | | sigmoid | affected | ++ | + |
| CD45un | 37 | F | colonic | 12 | A. colon | unaffected | − | − |
| CD45aff | | | | | cecum | unaffected | − | + |
| CD48un1 | 44 | M | ileal | 20 | T. colon | unaffected | − | − |
| CD48un2 | | | | | T. colon | affected | − | − |
| CD49un | 21 | F | ileocolonic | 3 | rectum | unaffected | − | + |
| CD49aff | | | | | cecum | affected | ++ | + |
| CD51un | 39 | M | colonic | 15 | SF. colon | unaffected | + | − |
| CD51aff | | | | | sigmoid | affected | ++ | + |
| CD53un | 55 | M | colonic | 15 | T. colon | unaffected | − | − |
| CD53aff | | | | | rectum | affected | +++ | ++ |
| CD58un | 51 | F | colonic | 10 | D. colon | unaffected | − | − |
| CD58aff | | | | | SF. colon | affected | + | + |
| CD59un | 32 | M | ileocolonic | 15 | D. colon | unaffected | − | − |
| CD59aff | | | | | T. colon | affected | − | − |
| CD76un | 76 | M | colonic | 2 | rectum | unaffected | − | − |
| CD76aff1 | | | | | sigmoid | affected | ++ | ++ |
| CD76aff2 | | | | | sigmoid | affected | ++ | ++ |
| Mean | 42.1 | | | 12 | | | | |
| Range | 21-76 | | | 2-20 | | | | |
| UC32un | 82 | F | colonic | 15 | A. colon | unaffected | − | + |
| UC32aff | | | | | rectum | affected | + | ++ |
| UC35un | 40 | F | colonic | 24 | A. colon | unaffected | − | − |
| UC35aff | | | | | rectum | affected | ++ | ++ |
| UC38un | 60 | M | colonic | 12 | A. colon | unaffected | − | − |
| UC38aff | | | | | sigmoid | affected | + | − |
| UC44un | 45 | M | colonic | 10 | D. colon | unaffected | − | − |
| UC44aff | | | | | sigmoid | affected | + | − |
| UC55un | 64 | F | colonic | 46 | HF. | unaffected | − | − |
| | | | | | colon | | | |
| UC55aff | | | | | rectum | affected | ++ | ++ |
| Mean | 58.2 | | | 15 | | | | |
| Range | 45-82 | | | 10-46 | | | | |
| N65 | 22 | F | | | sigmoid | normal | − | − |
| N66 | 64 | M | | | sigmoid | normal | | |
| N69 | 65 | F | | | sigmoid | normal | − | − |
| N79 | 57 | F | | | sigmoid | normal | − | − |
| Mean | 52 | | | | | | | |
| Range | 22-64 | | | | | | | |

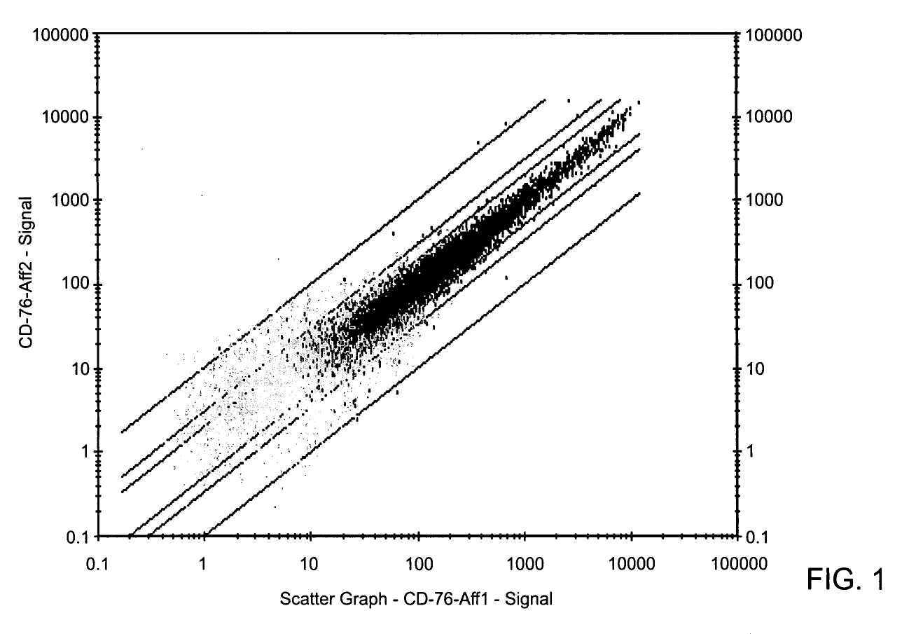

*CD: Crohn's disease, UC: ulcerative colitis, un: unaffected, aff: affected, N: normal control,
**Site of biopsy: T: transverse, A: ascending, D: descending, SF: splenic flexure, HF: hepatic flexure.
#Score based on active (polymorphonuclear) and chronic (lymphoplasmacytic) inflammation.
##Fibrosis score based on extent of lamina propria involvement, splaying of the muscularis mucosa, and crypt dropout (Supplemental FIG. 1)

Endoscopic Pinch Biopsies

“Affected” pinch biopsies are from areas appearing affected by endoscopy, “unaffected” biopsies are from an area at least 10 cm away from any grossly diseased area (Table 10). For every microarray sample, histology of an adjacent biopsy was scored for inflammation and fibrosis (A.M.) (Table 10). A four-tier grading scheme (−, +, ++, +++), based on semi-quantitative assessment of mucosal inflammation and fibrosis was used.

RNA Isolation and Microarray

Each biopsy, approximately 2×2×3 mm³ and weighing 2-7 mg (mean=4.7 mg, n=6 biopsies), produced ˜5 μg total RNA (TRIzol Reagent, Invitrogen Co), yielding 15 μg of biotin-labeled cRNA (https://www.affymetrix.com/support/technical/manual/). Biotinylated cRNA (10 μg per array) was hybridized to high-density oligonucleotide GeneChip Human Genome U95Av2 arrays (Affymetrix). The arrays were washed and stained (R-Phycoerythrin Streptavidin) in a GeneChip Fluidics Station 400. Images captured in a HP GeneArray Scanner (Affymetrix) were analyzed first by Microarray Suite 5.0 software (Affymetrix). Each transcript received a “present” or “absent” call based on whether the gene transcript was detected in the sample. The background intensities were low (40±0.6 to 52±1.0 arbitrary units), with ˜48.4% to 56.9% of all 12,625 probe sets marked as “present” in the biopsy samples, consistent with our previous study of whole colon tissue resections.⁷

Data Analysis

The DNA-Chip Analyzer (dChip) software¹⁷ was used to normalize the data from the image files for array-to-array comparisons (http://www.ncbi.nlm.nih.gov/geo). We used (1) Significance Analysis of Microarrays (SAM) software,¹⁸ to select biologically significant changes in gene expression between groups using the criteria of median FDR ≦0.1%, fold change >2, and Log₂ mean expression index >5.64, and (2) classical Multidimensional Scaling (MDS),¹⁹ that provides a low dimensional, distance-preserving map such that arrays with similar profiles are close on the map, to visualize the data and relationships between samples.

On comparing gene expression patterns of 2 biopsies, 10 cm apart, from within an affected area of one CD patient (CD-76-aff1 and CD-76-aff2), only 10 genes showed >2 fold difference in expression (from 3384 “present” genes)—an error of 0.29% in independent gene expression measurements of the same affected area. Thus, one endoscopic biopsy is considered a reliable representation of the disease (FIG. 1).

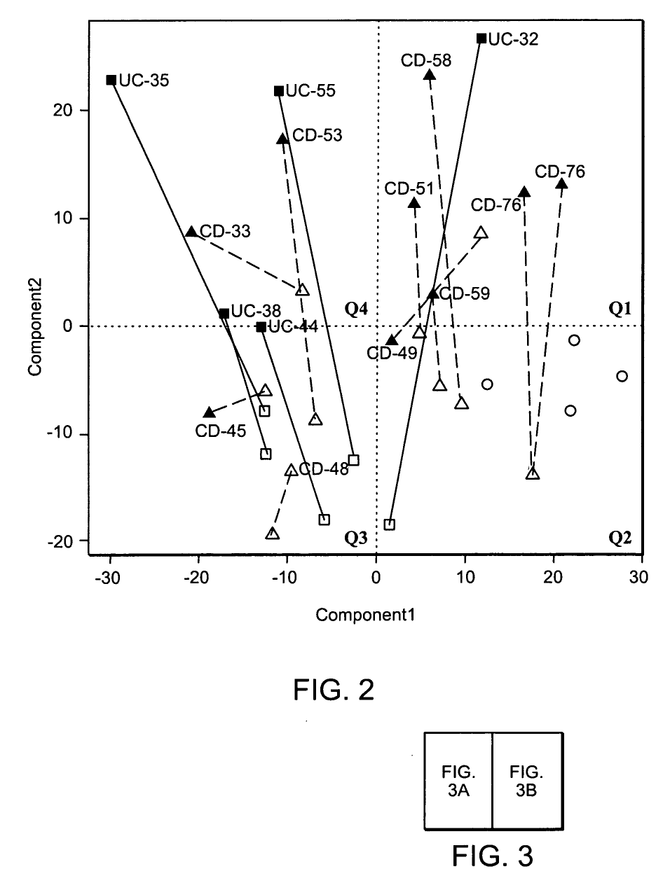

Analysis of 32 samples by MDS (FIG. 2), placed 11 of 13 affected IBD biopsies above the horizontal axis, in quadrants Q 1 and Q4 separated from unaffected and healthy control samples. Most unaffected and control biopsies (17/19) are below the horizontal axis in Q2 and Q3. Second, UC affected clearly separate from CD affected, except one (UC-32), that by histology showed mild inflammation only and fibrosis of 2+ grade. Among the CD cases, 5 biopsies with active disease appear together in Q1; clinically these have colonic involvement, characterized by rectal sparing. Two other patients (CD-33 and CD-53) with rectal disease and high histopathologic inflammation scores co-localized with the UC affected, possibly representing a CD subgroup resembling UC. CD-45 affected endoscopically, placed in the MDS plot with controls and unaffected was subsequently found to be negative for inflammation and fibrosis by histology.

To determine the biological differences in samples driving the MDS distribution, genes were sought that were responsible for positioning of the samples in the different quadrants of the MDS map. An analysis of variance on each gene identified those with significant, quadrant specific differences in expression. From the expression pattern of these genes (FIG. 3), three groups are evident. (Group 1): Twenty-seven genes expressed above mean in the controls and in 5 CD individuals are down regulated in four of the five individuals with UC. A majority of these genes code for membrane-bound endoplasmic reticulum-, Golgi apparatus-, or in a few cases lysozomal-proteins. These are primarily epithelial genes that regulate protein trafficking and secretion. The only two CD individuals that manifest this UC pattern are CD 33 and 53, both noted for active rectal inflammation resembling UC. (Group 2): Nine genes are elevated in most CD and UC affected profiles and most likely contribute towards separation of IBD from normal controls in the MDS plot. These genes include several chemokine ligands produced by activated monocytes and neutrophils, indicative of an immune/inflammation process and seem to correlate well with the inflammation scoring of the samples by histology. (Group 3): Thirteen genes are over expressed in UC primarily and the two UC-like CD cases 33 and 53, roughly distinguishing UC from CD (FIG. 3).

Significance analyses of microarrays (SAM) to compare affected to normal controls to identify a consistent expression pattern for diseased CD and UC tissues. CD cases confirmed to have active disease by histology were included, CD-45, -48 and -49 with inactive disease and distanced from the other CD cases by MDS were excluded. The CD-unique expression pattern highlights biological processes believed to play major roles in CD pathogenesis (Table 5). These include inflammatory response (IL1B, S100A8), antigen presentation (MHC class II immunoproteasome members PSME2 and PSMB8, MHC class II ATP-binding antigen peptide transporter TAP1, HLA-DMA and UBD of MHC class I), inflammatory cell chemotaxis (IL8, CXCL1, CXCL3), apoptosis (CASP1, CASP10), macrophage activation (ASMT and interferon-regulated genes IFITM1, IFITM3, ISG20, IFI35, SP110), leukocyte protection (LILRB encoding a receptor for class I MHC antigens), and acute phase response (ADM, STAT1, STAT3, and protease inhibitors SERPINA1 and SPINK1 to prevent tissue destruction). Certain overlaps evident between the CD and the UC over expressed gene signatures (Table 2. lower panel), involve immune response, antigen presentation (IGHG4, GIP3, LCN2), complement function (C4BPB, DAF), antimicrobial (DEFA6) and general inflammatory response (NOS2A, S100A9, REG1A, PAP).

TABLE 11
CD Gene Expression Signature

| Gene | Symbol | Biological function | Cytoband |
|---|---|---|---|
| CD unique gene expression* | | | |
| Adrenomedullin** | ADM | Acute phase response | 11p15.4 |
| Serine protease inhibitor, Kazal type 1 | SPINK1 | Acute phase response | 5q32 |
| Serine/cysteine proteinase inhibitor, clade A, 1 | SERPINA1 | Acute phase response | 14q32.1 |
| Signal transducer and activator of | STAT1 | Acute phase response | 2q32.2 |
| transcription 1 | | | |
| Signal transducer and activator of | STAT3 | Acute phase response | 17q21.31 |
| transcription 3** | | | |
| Proteasome activator subunit 2** | PSME2 | Antigen presentation | 14q11.2 |
| Proteasome subunit, beta type, 8** | PSMB8 | Antigen presentation | 6p21.3 |
| Ubiquitin D | UBD | Antigen presentation | 6p21.3 |
| Ubiquitin-conjugating enzyme E2L 6 | UBE2L6 | Antigen presentation | 11q12 |
| Transporter 1, ATP-binding cassette, sub B | TAP1 | Antigen presentation | 6p21.3 |
| Caspase 1 | CASP1 | Apoptosis | 11q23 |
| Caspase 10 | CASP10 | Apoptosis | 2q33-q34 |
| Acetylserotonin O-methyltransferase | ASMT | B-cell activation | Xp22.3/Yp11.3 |
| Mucin 1, transmembrane | MUC1 | Cytoskeleton | 1q21 |
| Myosin, light polypeptide 3 | MYL3 | Cytoskeleton | 3p21.3-p21.2 |
| Chymotrypsin-like | CTRL | Immune response | 16q22.1 |
| Interferon induced transmembrane protein 1 | IFITM1 | Immune response | 11p15.5 |
| Interferon induced transmembrane protein 3 | IFITM3 | Immune response | 11p15.5 |
| Interferon stimulated gene 20 kDa | ISG20 | Immune response | 15q26 |
| Interferon-induced protein 35** | IFI35 | Immune response | 17q21 |
| Interleukin 1, beta | IL1B | Immune response | 2q14 |
| Leukocyte Ig-like receptor, subfamily B, 1 | LILRB1 | Immune response | 19q13.4 |
| MHC, class II, DM alpha | HLA-DMA | Immune response | 6p21.3 |
| SP110 nuclear body protein | SP110 | Immune response | 2q37.1 |
| Chemokine (C—X—C motif) ligand 1** | CXCL1 | Inflammatory cell | 4p21 |
| | | recruitment | |
| Chemokine (C—X—C motif) ligand 3 | CXCL3 | Inflammatory cell | 4q21 |
| | | recruitment | |
| Interleukin 8 | IL8 | Inflammatory cell | 4q13-q21 |
| | | recruitment | |
| Regenerating islet-derived 1 beta | REG1B | Inflammatory cell | 2p12 |
| | | recruitment | |
| S100 calcium binding protein A8 | S100A8 | Inflammatory cell | 1q21 |
| | | recruitment | |
| Lipase, gastric | LIPF | Lipid metabolism | 10q23.31 |
| Ig lambda variable (IV)/OR22-2 | IGLVIVOR22-2 | Unknown | 22q12.2-q12.3 |
| Gene expression common to CD and UC* | | | |
| Ig heavy constant gamma 4 (G4m marker) | IGHG4 | Antigen binding | 14q32.33 |
| Defensin, alpha 6, Paneth cell-specific | DEFA6 | Antimicrobial | 8pter-p21 |
| Complement component 4 binding protein, β | C4BPB | Complement cascade | 1q32 |
| Decay accelerating factor for complement | DAF | Complement regulation | 1q32 |
| Membrane-associated protein 17 | MAP17 | Epithelial cell proliferation | 1p33 |
| Chemokine (C—X—C motif) ligand 2 | CXCL2 | Immune response | 4q21 |
| Deleted in malignant brain tumors 1** | DMBT1 | Immune response | 10q25.3-q26.1 |
| Interferon, alpha-inducible protein | G1P3 | Immune response | 1p35 |
| Lipocalin 2 | LCN2 | Inflammatory response | 9q34 |
| Nitric oxide synthase 2A | NOS2A | Inflammatory response | 17q11.2-q12 |
| Pancreatitis-associated protein | PAP | Inflammatory response | 2p12 |
| Regenerating islet-derived 1 alpha | REG1A | Inflammatory response | 2p12 |
| S100 calcium binding protein A9 | S100A9 | Inflammatory response | 1q21 |
| Protein kinase C, eta | PRKCH | MAPK signaling | 14q22-q23 |
| Regulator of G-protein signalling 3 | RGS3 | MAPK signaling | 9q32 |
| DNA-damage-inducible transcript 4 | DDIT4 | Unknown | 10pter-q26.12 |
| Hypothetical protein FLJ12443 | FLJ12443 | Unknown | 5p15.33 |

*All genes listed here are up regulated compared to normal controls
**Expression confirmed by quantitative RT-PCR

In the UC signature, derived by comparing all five UC affected to control, up-regulations suggest complement cascade activation (BF and C4A), growth regulatory (MIA) and apoptosis (ATM) changes, detoxification (NNMT) and intracellular transport (SNX26) (Table 12). Down regulations in UC are seen in biosynthetic and metabolic processes (PANK3, HPGD), and in endoplasmic reticulum-, Golgi-transport/intracellular trafficking (F2RL1, GABRG3, GNGT1, SLC4A4).

TABLE 12
UC Gene Expression Signature

| Gene | Symbol | Biological function | Cytoband |
|---|---|---|---|
| Up-regulated | | | |
| Defensin, alpha 5, Paneth cell-specific | DEFA5 | Antimicrobial | 8pter-p21 |
| Ataxia telangiectasia mutated | ATM | Apoptosis | 11q22-q23 |
| Chemokine (C—X—C motif) ligand 13 | CXCL13 | B-cell chemoattractant | 4q21 |
| B-factor, properdin | BF | Complement activation | 6p21.3 |
| Complement component 4A | C4A | Complement activation | 6p21.3 |
| Actin, beta | ACTB | Cytoskeleton | 7p15-p12 |
| Nicotinamide N-methyltransferase | NNMT | Detoxification | 11q23.1 |
| Melanoma inhibitory activity | MIA | Growth regulation | 19q13.32-q13.33 |
| Sorting nexin 26 | SNX26 | Intracellular protein | 19q13.13 |
| | | transport | |
| A disintegrin and metalloproteinase domain 5 | ADAM5 | Unknown | 8p11.23 |
| RNA binding motif protein 8A | RBM8A | Unknown | 1q12 |
| Tribbles homolog 2 (Drosophila) | TRIB2 | Unknown | 2p25.1 |
| Down-regulated | | | |
| Cyclin G1 | CCNG1 | Cell growth | 5q32-q34 |
| Myeloid/lymphoid or mixed-lineage leukemia; translocated to, 3 | MLLT3 | Cell growth | 9p22 |
| Protein phosphatase 2 (formerly 2A), regulatory subunit B″, alpha | PPP2R3A | Cell growth regulation | 3q22.1 |
| Pantothenate kinase 3 | PANK3 | CoA biosynthetic | 5q34 |
| Dynein, axonemal, heavy polypeptide 9 | DNAH9 | Cytoskeleton | 17p12 |
| Guanine nucleotide binding protein, gamma transducing activity polypeptide 1 | GNGT1 | G protein member | 7q21.3 |
| Coagulation factor II (thrombin) receptor-like 1 | F2RL1 | Golgi apparatus protein | 5q13 |
| Surfactant, pulmonary-associated protein D | SFTPD | Innate immune response | 10q22.2-q23.1 |
| Solute carrier family 4, sodium bicarbonate cotransporter, member 4 | SLC4A4 | Ion transport | 4q21 |
| Gamma-aminobutyric acid (GABA) A receptor, gamma 3 | GABRG3 | Ligand-gated ion channel | 15q11-q13 |
| Hydroxyprostaglandin dehydrogenase 15-(NAD) | HPGD | Prostaglandin metabolism | 4q34-q35 |
| TAF5-like RNA polymerase II, p300/CBP-associated factor (PCAF)-associated | TAF5L | Transcription | 1q42.13 |
| factor, 65 kDa | | | |
| Protein kinase, cAMP-dependent, catalytic, beta | PRKACB | Wnt-signaling | 1p36.1 |

Global gene expression patterns were obtained from single endoscopic pinch biopsies that were reproducible and representative of the local diseased area. Overlap was found between profiles of resected tissues and endoscopic tissues. Both UC patterns are quite dynamic showing multiple gene expression changes (REG1A, LCN2, NOS2, NNMT, for example). In contrast, the signature for resected CD tissues was remarkably static compared to that of biopsies. The CD biopsy tissues show induction of several chemokine and interferon-γ responsive genes.

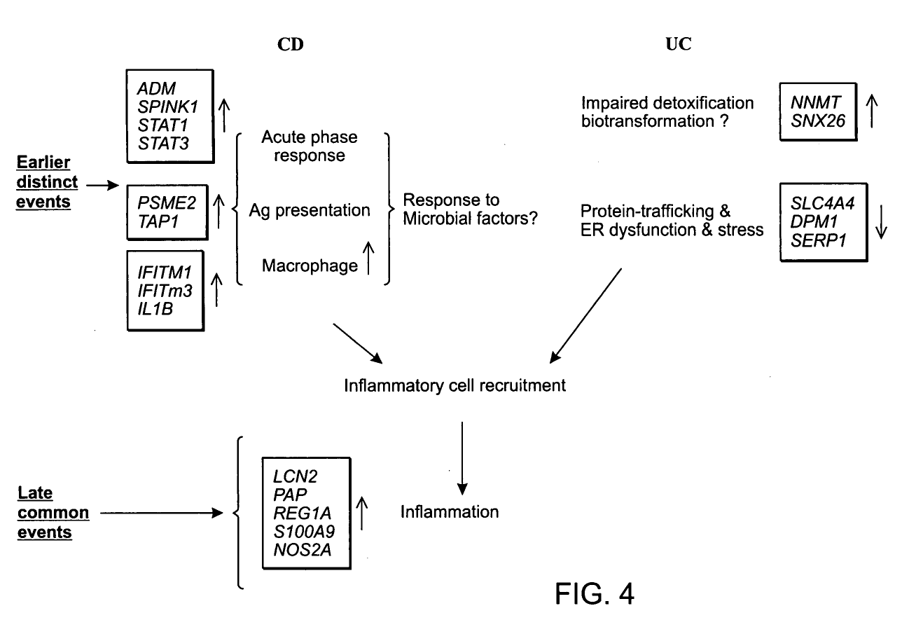

Without wishing to be bound by theory, gene expression differences in CD and UC speak of fundamentally different biological processes contributing to their pathogenesis. The genes over-expressed in CD are overwhelmingly those of acute phase and innate immune response (involving IL-1 and TNFα mediated induction of NF-κB), MHC class II mediated antigen presentation, macrophage activation and recruitment of inflammatory cells. The distinctive transmural tissue damage and mesenchymal involvement in CD may be due to this major early involvement of immune and inflammatory cells. Gene expression changes in UC, on the other hand, make a strong case for loss of epithelial homeostasis as being central to UC. Epithelial secretion is a process that is pivotal to maintaining intestinal mucosal integrity.²⁰ Intracellular trafficking and secretory functions of the endoplasmic reticulum (ER) are essential for the degradation and secretion of ingested environmental toxins by the intestinal epithelium. Upon examining the UC signature and the 50 genes whose expression differences coincide with separation of UC from CD in the MDS plot, it was observed that a number of genes functioning in epithelial secretion, intracellular trafficking and endoplasmic reticulum or Golgi functions are remarkably down regulated in UC. An overload of degraded, unfolded proteins has been proposed to cause ER stress as in the Ire1β (Inositol requiring kinase 1)—deficient mouse that develop colitis when challenged with dextran sodium sulfate.²¹ Without wishing to be bound by theory, initial events in CD and UC may be quite different (FIG. 4). In CD it is mostly a deregulation of immune functions as has been believed for a long time, while impaired detoxification and ER stress contribute to UC. Interestingly, ER stress has been recently linked to obesity, insulin resistance and type 2 diabetes.²² In that study metabolic and inflammatory stress (increased lipid synthesis) was suggested to cause increased workload in the ER. In UC down regulation of metabolic and biotransformation enzymes may be the primary cause of ER stress.

Unsupervised multidimensional scaling was used on the IBD and normal gene expression profiles to develop a systematic approach towards molecular classification²⁴⁻²⁸ of disease subtypes. There is a clear separation of controls from IBD. Within the CD cases there is a grouping of some into one sub group, with two other CD cases localizing with UC samples, underscoring the heterogeneous nature of CD.

The following genes from the signatures pose promising IBD candidate genes: apoptosis-regulating CASP10 at 2q33-34, LILRB1 at 19q13.4 (locus IBD6) and antigen-presenting genes PSME2 at 14q11.2 (locus IBD4). With respect to the IBD3 locus at 6p21,³⁵ HLA-DMA, TAP1, UBD and PSMB8 (immunoproteasome for generating MHC class I binding antigenic peptides), at 6p21.3, are particularly intriguing. GNGT1 (7q21.3) functioning in apoptosis and PRKACB (1p36.1, IBD7), involved in Wnt-signaling from the UC signature are also good candidates.

### Sample Classification by Multidimensional Scaling

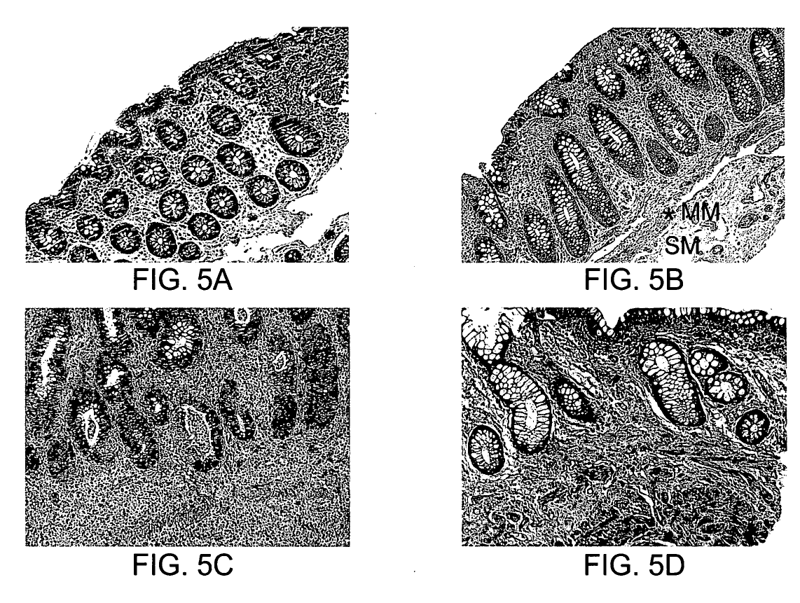

A total of 18 CD samples (8 affected and 10 unaffected biopsies), 10 UC samples (5 affected and 5 unaffected) and 4 normal biopsy samples were analyzed. The histological assessment of the biopsy samples are presented first to evaluate the MDS classification in the context of their histology. Control and unaffected biopsies essentially display normal colonic architecture, with no evidence of cryptitis, crypt distortion, or acute and chronic inflammation (FIG. 5, A and B). In contrast, biopsies marked as “affected” manifest variable degrees of acute or chronic colitis, including one or more of the following histologic features: cryptitis, with or without accompanying crypt abscesses, crypt distortion, lamina propria fibrosis, crypt dropout, basal lymphoplasmacytosis, and Paneth cell metaplasia (FIG. 5, C and D). None of the biopsies indicate evidence of colitis-associated epithelial dysplasia or neoplasia.

Cross Validation by Quantitative RT-PCR (qRT-PCR)

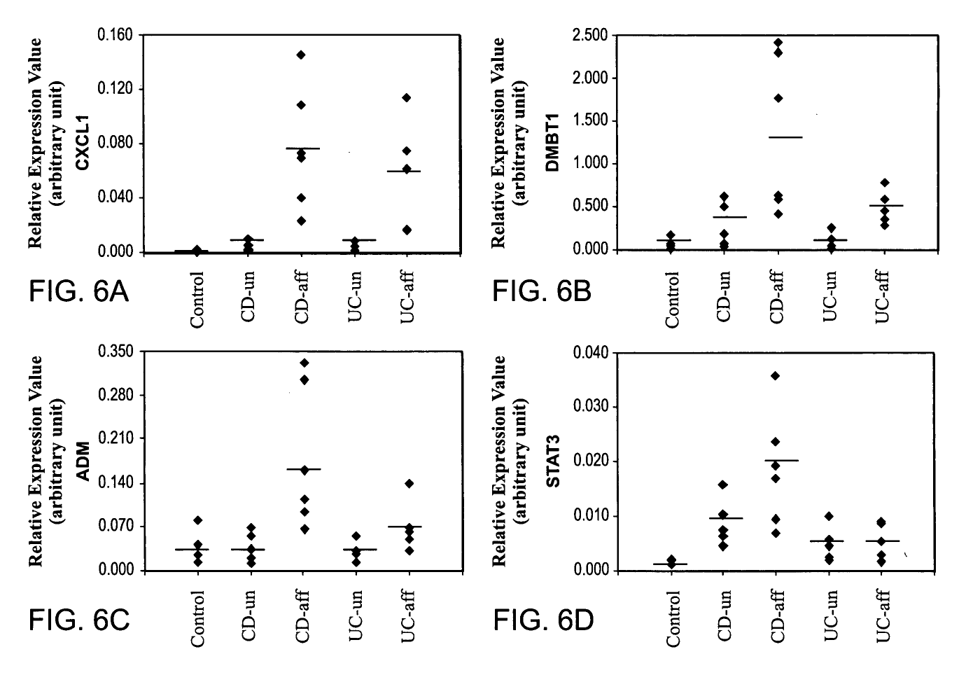

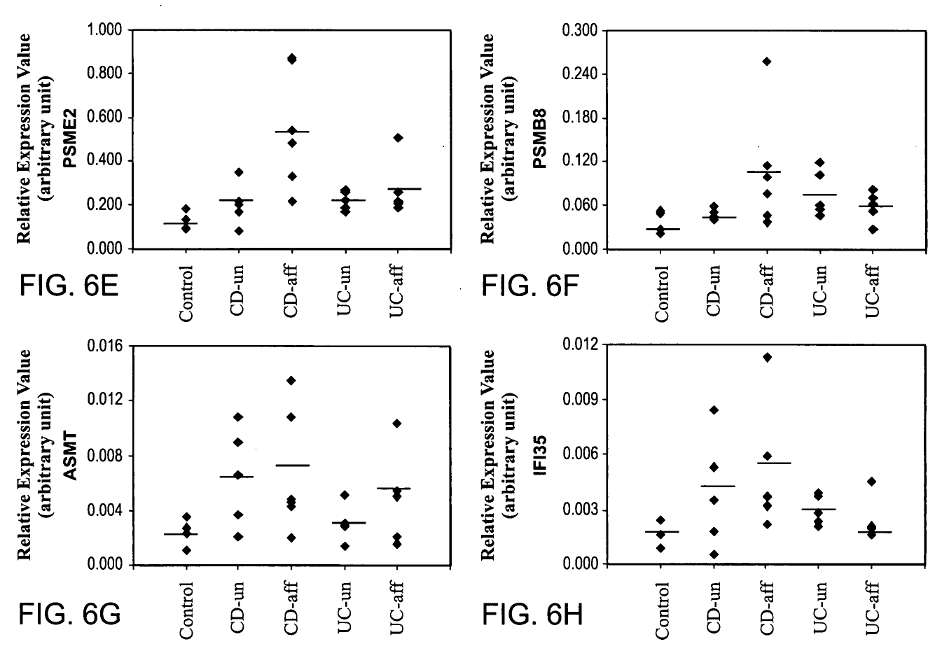

Eight genes (CXCL1, DMBT1, ASMT, ADM, STAT3, IFI35, PSME2 and PSMB8) were selected from our microarray expression profiles for further confirmation by quantitative (q) RT-PCR (FIG. 6). The qRT-PCR results show excellent agreement with the array analysis results. CXCL1 and DMBT1 are up regulated in CD and UC affected biopsies, while ADM, STAT3, PSME2 and PSMB8 are primarily up regulated in CD affected biopsies. ASMT and IFI35 show elevated levels of transcript in CD affected and unaffected biopsies (FIG. 6).

TABLE 13

| | | Probe SEQ ID NOS |
|---|---|---|
| | | (respectively, in |
| Sequence | SEQ ID NO | order of appearance) |
| PSME2 | 1 | 2-17 |
| PRKCH | 18 | 19-34 |
| G1P3 | 35 | 36-51 |
| IL8 | 52 | 53-68 |
| IL1B | 69 | 70-85 |
| CCNG1 | 86 | 87-102 |
| NOS2A | 103 | 104-119 |
| ATM | 120 | 121-136 |
| GABRG3 | 137 | 138-153 |
| EST (MAP17) | 154 | 155-170 |
| SFTPD | 171 | 172-187 |
| LCN2 | 188 | 189-204 |
| ISG20 | 205 | 206-221 |
| STAT1 | 222 | 223-238 |
| CXCL3 | 239 | 240-255 |
| DEFA6 | 256 | 257-272 |
| DEFA5 | 273 | 274-289 |
| ADM | 290 | 291-306 |
| TAF5L | 307 | 308-323 |
| LIPF | 324 | 325-340 |
| EST (SNX26) | 341 | 342-357 |
| ASMT | 358 | 359-374 |
| PANK3 | 375 | 376-391 |
| SP110 | 392 | 393-408 |
| BF | 409 | 410-425 |
| SLC4A4 | 426 | 427-442 |
| AMAD5 | 443 | 444-459 |
| LILRB1 | 460 | 461-476 |
| MLLT3 | 477 | 478-493 |
| REG1B | 494 | 495-510 |
| PRKACB | 511 | 512-527 |
| F2RL1 | 528 | 529-544 |
| DNAH9 | 545 | 546-561 |
| GNGT1 | 562 | 563-578 |
| SERPINA1 | 579 | 580-595 |
| NNMT | 596 | 597-612 |
| CXCL2 | 613 | 614-629 |
| EST (HPGD) | 630 | 631-646 |
| HLA-DMA | 647 | 648-663 |
| RGS3 | 664 | 665-680 |
| IGHG1 | 681 | 682-697 |
| C4BPB | 698 | 699-714 |
| SPINK1 | 715 | 716-731 |
| REG1A | 732 | 733-748 |
| MUC1 | 749 | 750-765 |
| EST | 766 | 767-782 |
| MIA | 783 | 784-799 |
| DAF | 800 | 801-816 |
| STAT3 | 817 | 818-833 |
| DDIT4 | 834 | 835-850 |
| PAP | 851 | 852-867 |
| UBD | 868 | 869-884 |
| CASP10 | 885 | 886-901 |
| TAP1 | 902 | 903-918 |
| PSKH1 | 919 | 920-935 |
| UBE2L6 | 936 | 937-952 |
| C4A | 953 | 954-969 |
| RBM8A | 970 | 971-986 |
| CXCL1 | 987 | 988-1003 |
| S100A8 | 1004 | 1005-1020 |
| CXCL13 | 1021 | 1022-1037 |
| EST (FLJ12443) | 1038 | 1039-1054 |
| PSMB8 | 1055 | 1056-1071 |
| DMBT1 | 1072 | 1073-1088 |
| S100A9 | 1089 | 1090-1105 |
| MYL3 | 1106 | 1107-1122 |
| IFITM3 | 1123 | 1124-1139 |
| IFI35 | 1140 | 1141-1156 |
| CASP1 | 1157 | 1158-1173 |
| IFITM1 | 1174 | 1175-1190 |
| TRIB2 | 1191 | 1192-1207 |
| PPP2R3A | 1208 | 1209-1224 |
| ACTB | 1225 | 1226-1245 |

| Accession Number | SEQ ID NO |
|---|---|
| NM_001124.1 | 1246 |
| NM_003122.2 | 1247 |
| NM_000295.3 | 1248 |
| NM_007315.2 | 1249 |
| NM_139276.2 | 1250 |
| NM_002818.2 | 1251 |
| NM_004159.3 | 1252 |
| NM_006398.2 | 1253 |
| NM_004223.3 | 1254 |
| NM_000593.5 | 1255 |
| NM_033292.1 | 1256 |
| NM_032974.2 | 1257 |
| NM_004043.1 | 1258 |
| NM_182741.1 | 1259 |
| NM_000258.1 | 1260 |
| NM_001907.1 | 1261 |
| NM_003641.2 | 1262 |
| NM_021034.1 | 1263 |
| NM_002201.4 | 1264 |
| NM_005533.2 | 1265 |
| NM_000576.2 | 1266 |
| NM_006669.2 | 1267 |
| NM_006120.2 | 1268 |
| NM_080424.1 | 1269 |
| NM_001511.1 | 1270 |
| NM_002090.1 | 1271 |
| NM_000584.2 | 1272 |
| NM_006507.2 | 1273 |
| NM_002964.3 | 1274 |
| NM_004190.1 | 1275 |
| AL021937.1 | 1276 |
| BC025985.1 | 1277 |
| NM_001926.2 | 1278 |
| NM_000716.2 | 1279 |
| NM_000574.2 | 1280 |
| NM_005764.3 | 1281 |
| NM_002089.1 | 1282 |
| NM_007329.1 | 1283 |
| NM_022873.1 | 1284 |
| NM_005564.2 | 1285 |
| NM_000625.3 | 1286 |
| NM_002580.1 | 1287 |
| NM_002909.3 | 1288 |
| NM_002965.2 | 1289 |
| NM_006255.3 | 1290 |
| NM_144488.1 | 1291 |
| NM_019058.1 | 1292 |
| NM_024830.3 | 1293 |
| NM_021010.1 | 1294 |
| NM_000051.2 | 1295 |
| NM_006419.1 | 1296 |
| NM_001710.3 | 1297 |
| NM_007293.1 | 1298 |
| NM_001101.2 | 1299 |
| NM_006169.1 | 1300 |
| NM_006533.1 | 1301 |
| NM_052948.2 | 1302 |
| NR_001448.1 | 1303 |
| NM_005105.2 | 1304 |
| NM_021643.1 | 1305 |
| NM_004060.3 | 1306 |
| NM_004529.1 | 1307 |
| NM_002718.3 | 1308 |
| NM_024594.2 | 1309 |
| NM_001372.2 | 1310 |
| NM_021955.2 | 1311 |
| NM_005242.3 | 1312 |
| NM_003019.3 | 1313 |
| NM_003759.1 | 1314 |
| NM_033223.1 | 1315 |
| NM_000860.3 | 1316 |
| NM_014409.2 | 1317 |
| NM_002731.2 | 1318 |

### REFERENCES

1. Fiocchi C. Inflammatory bowel disease: etiology and pathogenesis. Gastroenterology 1998; 115:182-205.
2. Podolsky D K. Inflammatory bowel disease. N Engl J Med 2002; 347:417-29.
3. Sartor R. Current concepts of the etiology and pathogenesis of ulcerative colitis and Crohn's disease. Gastroenterology Clinics of North America 1995; 24:475-507.
4. Blumberg R S, Strober W. Prospects for research in inflammatory bowel disease. Jama 2001; 285:643-7.
5. Marion J, Rubin P, Present D. Differential diagnosis of chronic ulcerative colitis and Crohn's disease. W. B. Saunders Company, 2000.
6. Sands B E. From symptom to diagnosis: clinical distinctions among various forms of intestinal inflammation. Gastroenterology 2004; 126:1518-32.
7. Lawrance I C, Fiocchi C, Chakravarti S. Ulcerative colitis and Crohn's disease: distinctive gene expression profiles and novel susceptibility candidate genes. Hum Mol Genet 2001; 10:445-56.
8. Dooley T P, Curto E V, Reddy S P, Davis R L, Lambert G W, Wilborn T W, Elson C O. Regulation of gene expression in inflammatory bowel disease and correlation with IBD drugs: screening by DNA microarrays. Inflamm Bowel Dis 2004; 10:1-14.
9. Uthoff S M, Eichenberger M R, Lewis R K, Fox M P, Hamilton C J, McAuliffe T L, Grimes H L, Galandiuk S. Identification of candidate genes in ulcerative colitis and Crohn's disease using cDNA array technology. Int J Oncol 2001; 19:803-10.
10. Dieckgraefe B, Stenson W, JR Korzenik, Swanson P, Harrington C. Analysis of mucosal gene expression in inflammatory bowel disease by parallel oligonucleotide arrays. Physiol Genomics 2000; 4:1-11.
11. Langmann T, Moehle C, Mauerer R, Scharl M, Liebisch G, Zahn A, Stremmel W, Schmitz G. Loss of detoxification in inflammatory bowel disease: dysregulation of pregnane X receptor target genes. Gastroenterology 2004; 127:26-40.
12. Bayless T M, Tokayer A Z, Polito J M, 2nd, Quaskey S A, Mellits E D, Harris M L. Crohn's disease: concordance for site and clinical type in affected family members—potential hereditary influences. Gastroenterology 1996; 111:573-9.
13. Podolsky D K. Inflammatory bowel disease (1). N Engl J Med 1991; 325:928-37.
14. Picco M F, Bayless T M. Prognostic consideration in idiopathic inflammatory bowel disease. In: Kirsner J B, ed. Inflammatory Bowel Disease. 5th ed. Philadelphia: WB Saunders, 2000:765-780.
15. Hommes D W, van Deventer S J. Endoscopy in inflammatory bowel diseases. Gastroenterology 2004; 126:1561-73.
16. Colombel J-F, Grandbastien B, Gower-Rousseau C, Plegat S, Evrard J-P, Dupas J-L, Gendre J-P, Modigliani R, Belaiche J, Hostein J, Hugot J-P, VanKruiningen H, Cortot A. Clinical characteristics of Crohn's disease in 72 families. Gastroenterology 1996; 111:604-607.
17. Li C, Wong W H. Model-based analysis of oligonucleotide arrays: expression index computation and outlier detection. Proc Natl Acad Sci USA 2001; 98:31-6.
18. Tusher V G, Tibshirani R, Chu G. Significance analysis of microarrays applied to the ionizing radiation response. Proc Natl Acad Sci USA 2001; 98:5116-21.
19. Torgerson W S. Theory and Methods of Scaling. Wiley, 1958.
20. Chang E B. Intestinal Epithelial Function and Response to Mucosal Injury in Inflammatory Bowel Disease. In: Kirsner J B, ed. Inflammatory Bowel Disease. 5th ed. Philadelphia: WB Saunders, 2000:3-19.
21. Bertolotti A, Wang X, Novoa I, Jungreis R, Schlessinger K, Cho J H, West A B, Ron D. Increased sensitivity to dextran sodium sulfate colitis in IRE1beta-deficient mice. J Clin Invest 2001; 107:585-93.
22. Ozcan U, Cao Q, Yilmaz E, Lee A H, Iwakoshi N N, Ozdelen E, Tuncman G, Gorgun C, Glimcher L H, Hotamisligil G S. Endoplasmic reticulum stress links obesity, insulin action, and type 2 diabetes. Science 2004; 306:457-61.
23. Panwala C M, Jones J C, Viney J L. A novel model of inflammatory bowel disease: mice deficient for the multiple drug resistance gene, mdr1a, spontaneously develop colitis. J Immunol 1998; 161:5733-44.
24. Golub T, Slonim D, Tamayo P, Huard C, Gaasenbeek M, Mesirov J, Coller H, Loh M, Downing J, Caligiuri M, Bloomfield C, Lander E. Molecular classification of cancer: class discovery and class prediction by gene expression monitoring. Science 1999; 286:531-7.
25. Yeoh E J, Ross M E, Shurtleff S A, Williams W K, Patel D, Mahfouz R, Behm F G, Raimondi S C, Relling M V, Patel A, Cheng C, Campana D, Wilkins D, Zhou X, Li J, Liu H, Pui C H, Evans W E, Naeve C, Wong L, Downing J R. Classification, subtype discovery, and prediction of outcome in pediatric acute lymphoblastic leukemia by gene expression profiling. Cancer Cell 2002; 1:133-43.
26. Bittner M, Meltzer P, Chen Y, Jiang Y, Seftor E, Hendrix M, Radmacher M, Simon R, Yakhini Z, Ben-Dor A, Sampas N, Dougherty E, Wang E, Marincola F, Gooden C, Lueders J, Glatfelter A, Pollock P, Carpten J, Gillanders E, Leja D, Dietrich K, Beaudry C, Berens M, Alberts D, Sondak V. Molecular classification of cutaneous malignant melanoma by gene expression profiling. Nature 2000; 406:536-40.
27. Hedenfalk I, Duggan D, Chen Y, Radmacher M, Bittner M, Simon R, Meltzer P, Gusterson B, Esteller M, Kallioniemi O P, Wilfond B, Borg A, Trent J. Gene-expression profiles in hereditary breast cancer. N Engl J Med 2001; 344:539-48.
28. Fuller G N, Hess K R, Rhee C H, Yung W K, Sawaya R A, Bruner J M, Zhang W. Molecular classification of human diffuse gliomas by multidimensional scaling analysis of gene expression profiles parallels morphology-based classification, correlates with survival, and reveals clinically-relevant novel glioma subsets. Brain Pathol 2002; 12:108-16.
29. Vasiliauskas E A, Kam L Y, Karp L C, Gaiennie J, Yang H, Targan S R. Marker antibody expression stratifies Crohn's disease into immunologically homogeneous subgroups with distinct clinical characteristics. Gut 2000; 47:487-96.
30. Morley M, Molony C M, Weber T M, Devlin J L, Ewens K G, Spielman R S, Cheung V G. Genetic analysis of genome-wide variation in human gene expression. Nature 2004; 430:743-7.
31. Satsangi J, Parkes M, Louis E, Hashimoto L, Kato N, Welsh K, Terwilliger J, Lathrop G, Bell J, Jewell D. Two stage genome-wide search in inflammatory bowel disease provides evidence for susceptibility loci on chromosome 3, 7 and 12. Nature Genet 1996; 14:199-202.
32. Hugot J P, Thomas G. Genome-wide scanning in inflammatory bowel diseases. Dig Dis 1998; 16:364-9.
33. Brant S R, Shugart Y Y. Inflammatory bowel disease gene hunting by linkage analysis: rationale, methodology, and present status of the field. Inflamm Bowel Dis 2004; 10:300-11.
34. Cho J, Nicolae D, Gold L, Fields C, LaBuda M, Rohal P, Pickles M, Qin L, Fu Y, Mann J, Kirschner B, Jabs E, Weber J, Hanauer S, Bayless T, Brant S. Identification of novel susceptibility loci for inflammatory bowel disease on chromosomes 1p, 3q, and 4q: evidence for epistasis between 1p and IBD1. Proc Natl Acad Sci USA 1998; 95:7502-7.
35. Hampe J, Schreiber S, Shaw S H, Lau K F, Bridger S, Macpherson A J, Cardon L R, Sakul H, Harris T J, Buckler A, Hall J, Stokkers P, van Deventer S J, Nurnberg P, Mirza M M, Lee J C, Lennard-Jones J E, Mathew C G, Curran M E. A genomewide analysis provides evidence for novel linkages in inflammatory bowel disease in a large European cohort.PG-808-16. Am J Hum Genet 1999; 64:808-16.

### Example 2

Expressions of genes (PSME2, PSMB8, ADM, STAT3, CXCL1, DMBT1 and GAPDH) were quantified by real-time RT-PCR using QuantiTect SYBR Green PCR Kit (Qiagen Inc., Valencia, USA) according to manufacturer's instruction. The specific primers for genes selected are available online (supplementary Table 14). The relative expression value is defined as 2^{ΔC}^{_{T}}, where ΔC_{T}=(C_{T} of GAPDH−C_{T} of gene X)−(C_{T} of GAPDH no template control−C_{T} of gene X no template control).

TABLE 14
Primer for quantitative real-time RT-PCR

| Gene | | |
|---|---|---|
| symbol | Forward (5′→3′) | Reverse (5′→3′) |
| GAPDH | GTCTCCTCTGACTTCAACA | CAGGAAATGAGCTTGACAAA |
| PSME2 | ACCTGATCCCCAAGATTGAA | TGGAAATGGTTGTCTGGAAAG |
| PSMB8 | TAAGTCCAAGGAGAAGAAGAG | CAAATAGAGAACACGCAGAAGA |
| ADM | CAGCGAGGTGTAAAGTTG | GACTCGGTGTTTCCTTCTTC |
| DMBT1 | TGCTGTACTGACCTTGTTTG | GGGTCCGTAGGTGTCATC |
| CXCL1 | CCAAAGTGTGAACGTGAAG | TGGGGGATGCAGGATTGA |
| SATA3 | TTTTACCAAGCCCCCAAT | TGCTCGATGCTCAGTCCT |

### Immunohistochemistry

Immunohistochemistry was performed on paraffin-embedded sections of colonoscopic biopsies from 2 CD, 3 UC patients and 2 healthy controls (demographics included in supplementary Table 15). An ABC-staining kit with the rabbit anti-human TAP1 antibody (1 ug/ml) was used as described by the manufacturer (Santa Cruz Biotechnology, Santa Cruz, USA). The slides were counter-stained with Hematoxylin Gill No. 2 (Sigma).

TABLE 15
Demographics

| | | | Disease | | | | | | |
|---|---|---|---|---|---|---|---|---|---|
| | Age | | | Duration | Endoscopy | | Histology | | |
| Sample* | (years) | Sex | Location | (years) | Site | definition | Inflammation# | Fibrosis## | Medication** |
| CD138 | 40 | M | Ileocolonic | 17 | Sigmoid | Unaffected | +/− | − | 1, 3, 4 |
| CD141 | 45 | F | colonic | 13 | Splenic | Affected | ++ | − | 1, 3 |
| | | | | | flexure | | | | |
| Mean | 42.5 | | | | | | | | |
| UC133 | 52 | M | Pancolitis | 29 | Sigmoid | Unaffected | − | − | 1 |
| UC134 | 30 | F | Pancolitis | 21 | Sigmoid | Affected | ++ | − | 1, 2, 3, 4 |
| UC135 | 33 | F | Distal | 2 | Sigmoid | Unaffected | +/− | − | 1 |
| Mean | 38.3 | | | | | | | | |
| N145 | 56 | M | | | Sigmoid | normal | − | − | |
| N66 | 57 | F | | | Sigmoid | normal | − | − | |
| Mean | 56.5 | | | | | | | | |

*CD: Crohn's disease, UC: ulcerative colitis, N: healthy control.
#Score (−, +, ++, +++) based on active (polymorphonuclear) and chronic (lymphoplasmacytic) inflammation.
##Fibrosis score based on extent of lamina propria involvement, splaying of the muscularis mucosa, and crypt dropout.
**1 = 5ASA, 2 = antibiotics, 3 = steroids, 4 = immunomodulations: Azathioprine, 6MP, or Infliximab.

### Sample Classification by Multidimensional Scaling (MDS)

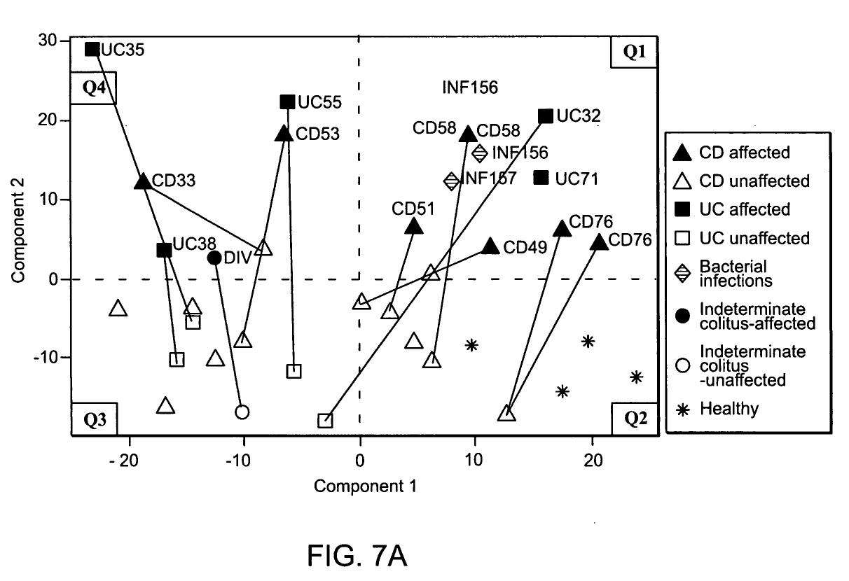

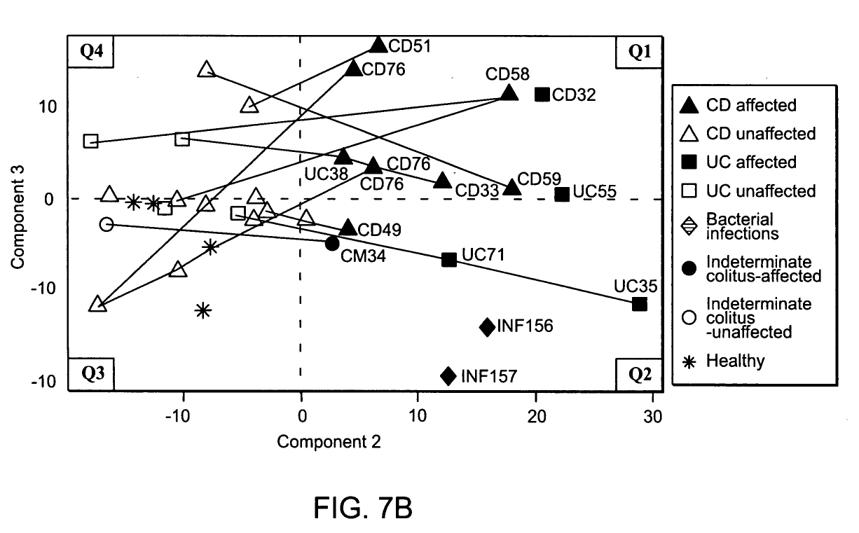

To explore the potential of classifying IBD types, based on gene expression patterns, we applied the unsupervised, no pre-defined groups, MDS clustering method on the entire microarray data set. An analysis of 36 samples by MDS indicated a clear distinction between sample-types (FIG. 7). First, all “affected” samples bearing inflammation appear in quadrants Q1 and Q4 (FIG. 7A. solid symbols), and separate clearly from unaffected and healthy control samples which appear in Q2 and Q3 (FIG. 7A. open symbols). When the following variables were plotted to the MDS map, we found that the distribution of samples in the MDS map is independent of the these variables: the sites from where biopsies were taken, patients' age, gender, disease duration, medication and fibrosis score of biopsy. Second, the majority of the CD cases appear together in Q1. Clinically these CD cases were characterized by rectal sparing. Two UC affected (UC-32 and UC-71) cases appearing in Q1, were diagnosed with pancolitis. On the other hand, three UC affected cases appear in Q4. Two CD cases (CD-33 and CD-53), with rectal disease and high histopathologic inflammation scores, co-localized with the UC affected in Q4, possibly representing a CD subgroup resembling UC. Finally, acute bacterial infectious colitis (INF156 and INF157) can be distinguished from IBD and diverticulitis in a second representation of the MDS map (FIG. 7B, MDS component 3 versus 2).

### Genes Differentially Expressed in CD and UC Affected Tissues

Significance Analyses of Microarrays (SAM) was used to identify genes that demonstrated consistent change in expression in affected CD and UC tissues versus healthy control (Table 16).

TABLE 16
Genes over-expressed in CD or UC affected tissues as compared with healthy
controls

| | Symbol | Biological implication | Cytoband |
|---|---|---|---|
| CD | | | |
| Adrenomedullin | ADM | Acute-phase response | 11p15.4 |
| Serum amyloid A1 | SAA1 | Acute-phase response | 11p15.1 |
| Serine/cysteine proteinase inhibitor, | SERPINA1 | Acute-phase response | 14q32.1 |
| clade A, 1 | | | |
| Signal transducer and activator of | STAT1 | Acute-phase response | 2q32.2 |
| transcription 1 | | | |
| Signal transducer and activator of | STAT3 | Acute-phase response | 17q21.31 |
| transcription 3 | | | |
| Leukocyte Ig-like receptor, subfamily | LILRB1 | Antigen binding | 19q13.4 |
| B, member 1 | | | |
| MHC, class II, DR beta 5 | HLA-DRB5 | Antigen presentation | 6p21.3 |
| Transporter 1, ATP-binding cassette, | TAP1 | Antigen processing | 6p21.3 |
| sub-family B | | | |
| Proteasome activator subunit 2 (PA28 | PSME2 | Antigen processing | 14q11.2 |
| beta) | | | |
| Proteasome subunit, beta type, 8 | PSMB8 | Antigen processing | 6p21.3 |
| Proteasome subunit, beta type, 9 | PSMB9 | Immune response | 6p21.3 |
| Proteasome subunit, beta type, 10 | PSMB10 | Immune response | 16q22.1 |
| Interferon, alpha-inducible protein | G1P3 | Immune response | 1p35 |
| (clone IFI-6-16) | | | |
| Interferon induced transmembrane | IFITM1 | Immune response | 11p15.5 |
| protein 1 (9-27) | | | |
| Interferon induced transmembrane | IFITM3 | Immune response | 11p15.5 |
| protein 3 (1-8U) | | | |
| Interferon stimulated gene 20 kDa | ISG20 | Macrophage activation | 15q26 |
| Caspase 10 | CASP10 | Apoptosis | 2q33-q34 |
| Mucin 4, tracheobronchial | MUC4 | Cell adhesion | 3q29 |
| Regenerating islet-derived 1 beta | REG1B | Cell proliferation | 2p12 |
| Mucin 1, transmembrane | MUC1 | Cytoskeleton | 1q21 |
| Serine protease inhibitor, Kazal type 4 | SPINK4 | Endopeptidase inhibitor | 9p13.3 |
| Lipin 1 | LPIN1 | Adipocyte differentiation | 2p25.1 |
| UC | | | |
| Coronin, actin binding protein, 1A | CORO1A | Cell migration | 16p11.2 |
| Matrix metalloproteinase 12 | MMP12 | Cell migration | 11q22.3 |
| Platelet/endothelial cell adhesion | PECAM1 | Cell migration | 17q23 |
| molecule (CD31) | | | |
| Talin 1 | TLN1 | Cell migration | 9p13 |
| Tissue inhibitor of metalloproteinase 1 | TIMP1 | Cell migration | Xp11.3-p11.23 |
| Interferon, gamma-inducible protein 30 | IFI30 | Immune response | 19p13.1 |
| POU domain, class 2, associating | POU2AF1 | Immune response, humoral | 11q23.1 |
| factor 1 | | | |
| Clusterin (complement lysis inhibitor, | CLU | Immune response/apoptosis | 8p21-p12 |
| SP-40,40) | | | |
| TNF receptor superfamily, member 7 | TNFRSF7 | Immune response/apoptosis | 12p13 |
| Prostaglandin D2 synthase | PTGDS | Inflammatory response | 9q34.2-q34.3 |
| CD79A antigen (Ig-associated alpha) | CD79A | Defense response | 19q13.2 |
| Defensin, alpha 5, Paneth cell-specific | DEFA5 | Antimicrobial response | 8pter-p21 |
| Ubiquitin D | UBD | Antimicrobial response | 6p21.3 |
| Chemokine (C-C motif) ligand 11 | CCL11 | Chemotaxis, eosinophil | 17q21.1-q21.2 |
| Insulin-like growth factor binding | IGFBP5 | Regulation of cell growth | 2q33-q36 |
| protein 5 | | | |
| Endothelial cell growth factor 1 | ECGF1 | Angiogenesis | 22q13 |
| (platelet-derived) | | | |
| Fascin homolog 1, actin-bundling | FSCN1 | Cell proliferation | 7p22 |
| protein | | | |
| Ataxia telangiectasia mutated | ATM | Apoptosis | 11q22-q23 |
| Notch homolog 3 (Drosophila) | NOTCH3 | Notch signaling | 19p13.2-p13.1 |
| Protease inhibitor 3, skin-derived | PI3 | Endopeptidase inhibitor | 20q12-q13 |
| (SKALP) | | | |
| Nucleoporin 210 | NIP210 | Development | 3p25.2-p25.1 |
| AT rich interactive domain 5A (MRF1- | ARID5A | DNA binding | 2q11.2 |
| like) | | | |
| Pyruvate dehydrogenase kinase, | PDK3 | Protein phosphorylation | Xp22.11 |
| isoenzyme 3 | | | |
| Cathepsin H | CTSH | Proteolysis | 15q24-q25 |
| Lymphocyte cytosolic protein 1 (L- | LCP1 | Unknown | 13q14.3 |
| plastin) | | | |
| Stomatin | STOM | Unknown | 9q34.1 |

Six CD affected cases were compared to healthy controls, while CD-45, -48 and -59 with biopsies demonstrating only inactive disease were excluded from this analysis. The CD expression pattern highlights biological processes believed to play major roles in CD pathogenesis. Proteins encoded by these 22 genes regulate antigen processing/presentation, macrophage activation and acute phase response (Table 16). A list of 12 genes down regulated in CD affected biopsies is presented in supplementary Table 17.

TABLE 17
Genes down-regulated in CD or UC as compared with healthy controls

| | Symbol | Biological implication | Cytoband |
|---|---|---|---|
| CD | | | |
| Down syndrome critical region gene 1-like 1 | DSCR1L1 | Calcium-mediated signaling | 6p21.1-p12.3 |
| Spondin 1, extracellular matrix protein | SPON1 | Cell adhesion | 11p15.2 |
| Thrombospondin 1 | THBS1 | Cell motility | 15q15 |
| Chemokine (C—X—C motif) ligand 12 | CXCL12 | Chemotaxis | 10q11.1 |
| Stathmin-like 2 | STMN2 | Neuron cell differentiation | 8q21.13 |
| Serine/cysteine proteinase inhibitor, clade B, 7 | SERPINB7 | Proteinase inhibitor | 18q21.33 |
| WEE1 homolog (S. pombe) | WEE1 | Regulation of cell cycle | 11p15.3-p15.1 |
| Myosin, heavy polypeptide 11, smooth muscle | MYH11 | Striated muscle contraction | 16p13.13-p13.12 |
| Chromosome 14 ORF116 (checkpoint | CHES1 | Transcription regulation | 14q24.3-q32.11 |
| suppressor 1) | | | |
| Pre-B-cell leukemia transcription factor 3 | PBX3 | Transcription regulation | 9q33-q34 |
| Autism susceptibility candidate 2 | AUTS2 | Unknown | 7q11.22 |
| Poliovirus receptor-related 3 | PVRL3 | Unknown | 3q13 |
| UC | | | |
| Semaphorin 6A-1 | SEMA6A | Apoptosis | 5q23.1 |
| KIAA0931 protein (PH domain and leucine rich | PHLPPL | Biosynthesis, cAMP | 16q22.2 |
| Repeat protein phosphatase-like) | | | |
| Mitochondrial ribosomal protein S6 | MRPS6 | Biosynthesis, protein | 21q21.3-q22.1 |
| Sterol-C5-desaturase (ERG3 delta-5-desaturase | SC5DL | Biosynthesis, steroid | 11q23.3 |
| Homolog, fungal)-like | | | |
| Related RAS viral (r-ras) oncogene homolog 2 | SCP2 | Biosynthesis, steroid | 11p15.2 |
| UDP-glucose dehydrogenase | UGDH | Biosynthesis, UDP-glucuronate | 4p15.1 |
| Calpastatin | CAST | calpain inhibitor activity | 5q15-q21 |
| ADAM-like, decysin 1 | ADAMDEC1 | cell adhesion inhibition | 8p21.2 |
| Dynein, axonemal, heavy polypeptide 9 | DNAH9 | cell motility | 17p12 |
| Ephrin-A1 | EFNA1 | cell-cell signaling | 1q21-q22 |
| Fibroblast growth factor receptor 3 | FGFR3 | MAPKKK/JAK-STAT cascade | 4p16.3 |
| Methylmalonyl Coenzyme A mutase | MUT | Metabolism | 6p21 |
| Phosphoenolpyruvate carboxykinase 1 (soluble) | PCK1 | Metabolism, gluconeogenesis | 20q13.31 |
| Gamma-glutamyl hydrolase | GGH | Metabolism, glutamine | 8q12.3 |
| N-acylsphingosine amidohydrolase-like | ASAHL | Metabolism, hydrolase activity | 4q21.1 |
| Acyl-Coenzyme A dehydrogenase, C-4 to C-12 | ACADM | Metabolism, lipid | 1p31 |
| straight chain | | | |
| UDP glycosyltransferase 2 family, B28 | UGT2B28 | Metabolism, lipid | 4q13 |
| Ectonucleoside triphosphate | ENTPD5 | Metabolism, neucleotide | 14q24 |
| diphosphohydrolase 5 | | | |
| Ectonucleotide | ENPP4 | Metabolism, nucleotide | 6p21.1 |
| pyrophosphatase/phosphodiesterase 4 | | | |
| Cisplatin resistance associated | MTMR11 | Metabolism, phospholipid | 1q12-q21 |
| aAcyl-Coenzyme A oxidase 1, palmitoyl | ACOX1 | Metabolism, prostaglandin | 17q24-q25 |
| Neural precursor cell expressed, | NEDD4L | Metabolism, ubiquitin-protein/ | 18q21 |
| developmentally down-regulated 4-like | | sodium transport | |
| Tetraspanin 7 (transmembrane 4 superfamily, 2) | TSPAN7 | N-linked glycosylation | Xp11.4 |
| Protein tyrosine phosphatase, receptor type, R | PTPRR | Protein dephosphorylation | 12q15 |
| Vacuolar protein sorting 13A (yeast) | VPS13A | Protein localization | 9q21 |
| Procollagen-lysine, 2-oxoglutarate 5- | PLOD2 | Protein modification | 3q23-q24 |
| dioxygenase 2 | | | |
| Dual-specificity tyrosine-(Y)-phosphorylation | DYRK2 | Protein phosphorylation | 12q15 |
| regulated kinase 2 | | | |
| Guanylate cyclase activator 2A (guanylin) | GUCA2A | Regulation of smooth muscle | 1p35-p34 |
| | | contraction | |
| Guanylate cyclase activator 2B (uroguanylin) | GUCA2B | Regulation of smooth muscle | 1p34-p13 |
| | | contraction | |
| Sorcin | SRI | Regulation of striated muscle | 7q21.1 |
| | | contraction | |
| Endothelin 3 | EDN3 | Regulation of vasoconstriction | 20q13.2-q13.3 |
| Peroxiredoxin 6 | PRDX6 | Response to oxidative stress | 1q25.1 |
| Selenium binding protein 1 | SELENBP1 | Selenium binding | 1q21-q22 |
| A kinase (PRKA) anchor protein (yotiao) 9 | AKAP9 | Signal transduction | 7q21-q22 |
| Phosphoinositide-3-kinase, regulatory subunit, | PIK3R1 | Signal transduction | 5q13.1 |
| polypeptide 1 (p85 alpha) | | | |
| Coagulation factor II (thrombin) receptor-like 1 | F2RL1 | Signal transduction/blood | 5q13 |
| | | coagulation | |
| Lectin, galactoside-binding, soluble, 2 (galectin | LGALS2 | Sugar binding | 22q13.1 |
| 2) | | | |
| Chromodomain helicase DNA binding protein 1 | CHD1 | Transcription regulation | 5q15-q21 |
| Hepatocyte nuclear factor 4, gamma | HNF4G | Transcription regulation | 8q21.11 |
| Myeloid/lymphoid or mixed-lineage leukemia | MLLT2 | Transcription regulation | 4q21 |
| (trithorax homolog, Drosophila); translocated | | | |
| to, 2 | | | |
| v-myb myeloblastosis viral oncogene homolog | MYB | Transcription regulation | 6q22-q23 |
| (avian) | | | |
| Nuclear receptor subfamily 3, group C, member 2 | NR3C2 | Transcription regulation | 4q31.1 |
| SATB family member 2 | SATB2 | Transcription regulation | 2q33 |
| Zinc finger protein 217 | ZNF217 | Transcription regulation | 20q13.2 |
| Cyclin T2 | CCNT2 | Transcription regulation | 2q21.3 |
| Kruppel-like factor 5 (intestinal) | KLF5 | Transcription regulation | 13q22.1 |
| ATPase, Ca++ transporting, plasma membrane 1 | ATP2B1 | Transport, calcium | 12q21.3 |
| Exophilin 5 | EXPH5 | Transport, protein | 11q22.3 |
| Solute carrier family 16, member 1 | SLC16A1 | Transport, organic anion | 1p12 |
| Secretory carrier membrane protein 1 | SCAMP1 | Transport, protein | 5q13.3-q14.1 |
| Transportin 1 | TNPO1 | Transport, protein | 5q13.2 |
| Solute carrier family 26, member 2 | SLC26A2 | Transport, sulfate | 5q31-q34 |
| Aquaporin 8 | AQP8 | Transport, water | 16p12 |
| Peptidyl arginine deiminase, type II | — | Unknown | 1p35.2-p35.1 |
| Cordon-bleu homolog (mouse) | COBL | Unknown | 7p12.1 |
| Family with sequence similarity 8, member A1 | FAM8A1 | Unknown | 6p22-p23 |
| Hypothetical protein FLJ13910 | FLJ13910 | Unknown | 2p11.2 |
| GRP1-binding protein GRSP1(FERM domain | FRMD4B | Unknown | 3p14.1 |
| containing 4B) | | | |
| Histone 1, H4c | HIST1H4C | Unknown | 6p21.3 |
| Hepatocellular carcinoma antigen gene 520 | LOC63928 | Unknown | 16p12.1 |
| Hypothetical protein LOC92482 | LOC92482 | Unknown | 10q24 |
| FLJ11220 (round spermatid basic protein 1) | RSBN1 | Unknown | 1p13.2 |

In the UC signature, derived by comparing all five UC affected to healthy controls, up-regulation of 26 genes suggests cell migration, growth regulatory and immune response changes as major pathogenic events (Table 16). Down regulations of 62 genes (supplementary Table 17) in UC include 16 genes encoding proteins that regulate biosynthetic and metabolic processes (UGDH, PCK1, GGH and others), 9 transcription regulation genes (CCNT2, CHD1, HNF4G, KLF5, MLLT2, MYB, NR3C2, SATB2, ZNF217), and 7 transporter genes (AQP8, EXPH5, SCAMP1, TNPO1, ATP2B1, SLC16A1, SLC26A2).

Overall, 25 genes were found to be up-regulated in both CD and UC, while 18 were down-regulated in both (Table 18). These genes are implicated in immune response (including antigen presentation, chemotaxis), general inflammatory response and cell proliferation or apoptosis. These may reflect inflammatory processes that are common to both disease types.

TABLE 18
Gene expression changes in CD and UC as compared with healthy controls

| | | Biological | |
|---|---|---|---|
| | Symbol | implication | Cytoband |
| Up-regulated | | | |
| Ig heavy constant gamma 4 (G4m marker) | IGHG4 | Antigen binding | 14q32.33 |
| MHC, class II, DM alpha | HLA-DMA | Antigen presentation | 6p21.3 |
| MHC, class II, DR beta 1 | HLA-DRB1 | Antigen presentation | 6p21.3 |
| Defensin, alpha 6, Paneth cell-specific | DEFA6 | Antimicrobial | 8pter-p21 |
| Chemokine (C—X—C motif) ligand 1 | CXCL1 | Chemotaxis | 4q21 |
| Chemokine (C—X—C motif) ligand 2 | CXCL2 | Chemotaxis | 4q21 |
| Chemokine (C—X—C motif) ligand 3 | CXCL3 | Chemotaxis | 4q21 |
| Interleukin 8 | IL8 | Chemotaxis | 4q13-q21 |
| B-factor, properdin | BF | Immune response | 6p21.3 |
| Decay accelerating factor for complement | DAF | Immune response | 1q32 |
| Deleted in malignant brain tumors 1 | DMBT1 | Immune response | 10q25.3-q26.1 |
| Lipocalin 2 (oncogene 24p13) | LCN2 | Acute-phase response | 9q34 |
| Nitric oxide synthase 2A (inducible, hepatocytes) | NOS2A | Inflammatory response | 17q11.2-q12 |
| Regenerating islet-derived 3 alpha | REG3A | Inflammatory response | 2p12 |
| S100 calcium binding protein A9 (MRP14) | S100A9 | Inflammatory response | 1q21 |
| Caspase 1 | CASP1 | Apoptosis | 11q23 |
| Peptidylprolyl isomerase D | PPID | Apoptosis | 4q31.1 |
| Pim-2 oncogene | PIM2 | Cell proliferation | Xp11.23 |
| Regenerating islet-derived 1 alpha | REG1A | Cell proliferation | 2p12 |
| Tryptophanyl-tRNA synthetase | WARS | Cell proliferation | 14q32.31 |
| | | inhibition | |
| Regulator of G-protein signalling 3 | RGS3 | Inactivation of MAPK | 9q32 |
| Hypothetical protein FLJ12443 | FLJ12443 | Muscle development | 5p15.33 |
| Protein serine kinase H1 | PSKH1 | Protein phosphorylation | 16q22.1 |
| Ubiquitin-conjugating enzyme E2L 6 | UBE2L6 | Ubiquitin cycle | 11q12 |
| PDZK1 interacting protein 1 | PDZK1IP1 | Unknown | 1p33 |
| Down-regulated | | | |
| Adducin 3 (gamma) | ADD3 | Calmodulin binding | 10q24.2-q24.3 |
| Claudin 8 | CLDN8 | Cell-cell adhesion | 21q22.11 |
| Protein kinase C, iota | PRKCI | Cell polarity | 3q26.3 |
| | | maintenance | |
| UDP glycosyltransferase 8 | UGT8 | Nervous development | 4q26 |
| BTB (POZ) domain containing 3 | BTBD3 | Protein binding | 20p12.2 |
| Protein kinase C-like 2 | PKN2 | Protein phosphorylation | 1p22.2 |
| Protein kinase, cAMP-dependent, catalytic, beta | PRKACB | Protein phosphorylation | 1p36.1 |
| ATP-binding cassette, sub-family B (MDR/TAP), 1 | ABCB1 | Transporter | 7q21.1 |
| Solute carrier family 4, member 4 | SLC4A4 | Transport, anion | 4q21 |
| MAX interactor 1 | MXI1 | Transcription regulation | 10q24-q25 |
| Sp3 transcription factor | SP3 | Transcription regulation | 2q31 |
| Frizzled-related protein | FRZB | Wnt receptor signaling | 2qter |
| Fk506-Binding Protein, Alt. Splice 2 | — | Unknown | — |
| mRNA; cDNA DKFZp586B211 | — | Unknown | — |
| Chromosome 14 open reading frame 11 | C14orf11 | Unknown | 14q13.1 |
| Creatine kinase, brain | CKB | Unknown | 14q132 |
| Transcribed sequences | KIAA1651 | Unknown | — |
| Putative MAPK activating protein | TIPRL | Unknown | 1q23.2 |

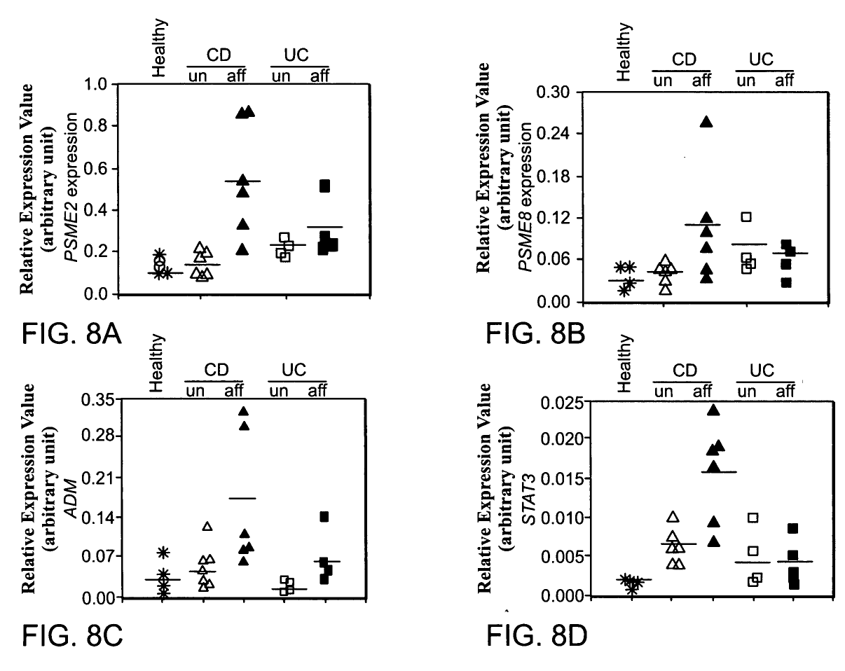

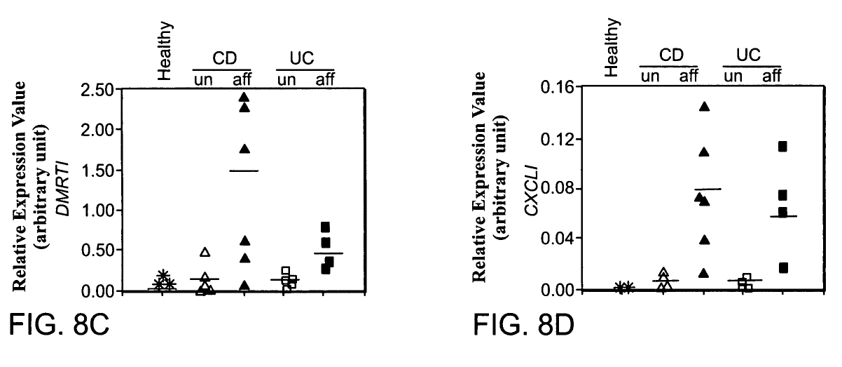

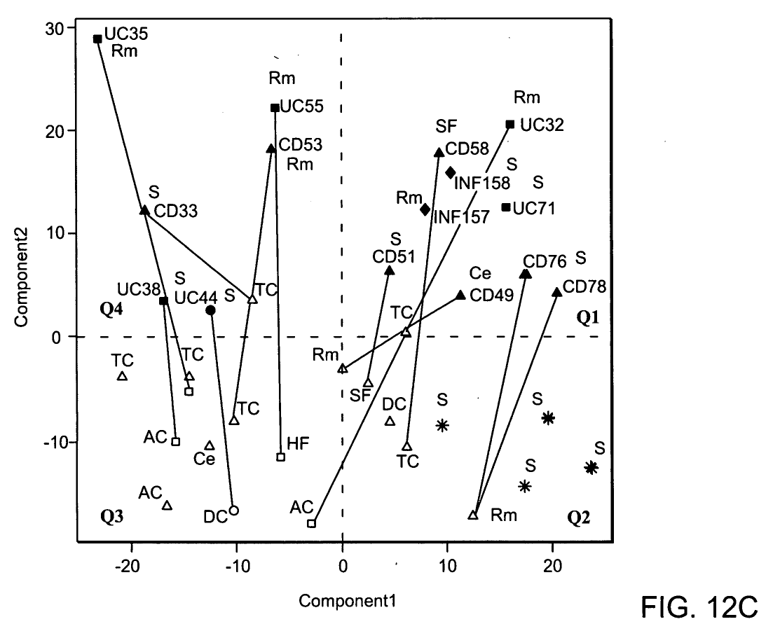

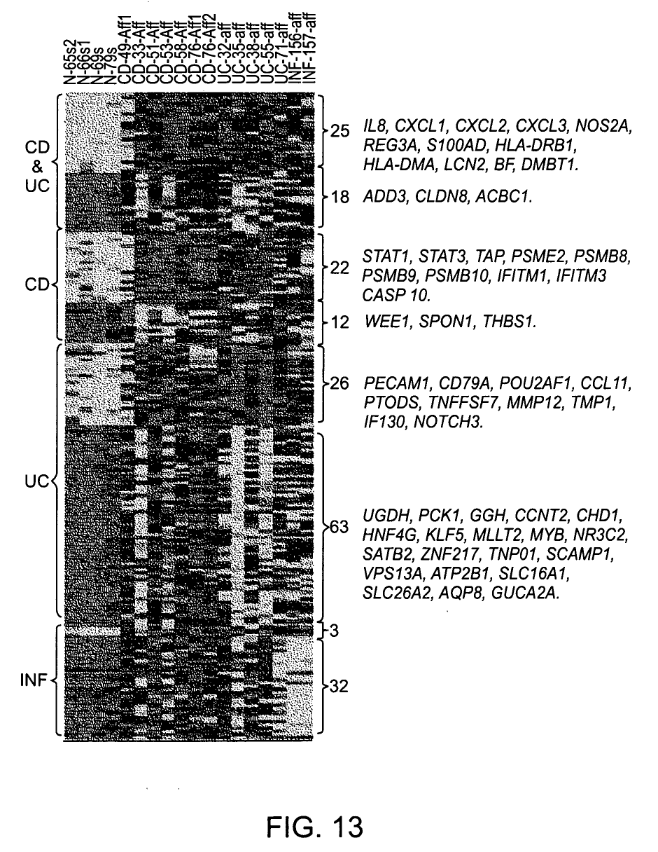

To confirm our microarray data, real-time RT-PCR was used to quantify the expression of genes including immunoproteasome subunit PSME2 and PSMB8, Adrenomedullin (ADM) and Signal transducer and activator of transcription 3 (STAT3) from Table 16, and Chemokine (C-X-C motif) ligand 1 (CXCL1) and Deleted in malignant brain tumors 1 (DMBT1) from Table 18. The mean expressions of PSME2, PSMB8, ADM and STAT3 were increased in CD affected biopsies, while CXCL1 and DMBT1 were incresded in both CD and UC affected compared to healthy control (FIG. 8). The results were corroborated the microarray data. In addition, expression of TAP1 (Transporter 1, ATP-binding cassette, sub-family B) protein was detected by immunohistochemistry on colon sections. The results demonstrated there were more TAP1-positive cells in the IBD samples than the healthy control, and immunopositive cells were more frequent in CD than UC (supplemental FIG. 13). TAP1 protein is expressed predominantly in intestinal macrophages (FIG. 13 B, arrows), and some crypt epithelial cells in the CD and UC affected biopsy tissues (FIGS. 13 B and D, arrowheads).

### Differences in Gene Expression Between IBD and Acute Bacterial Colitis

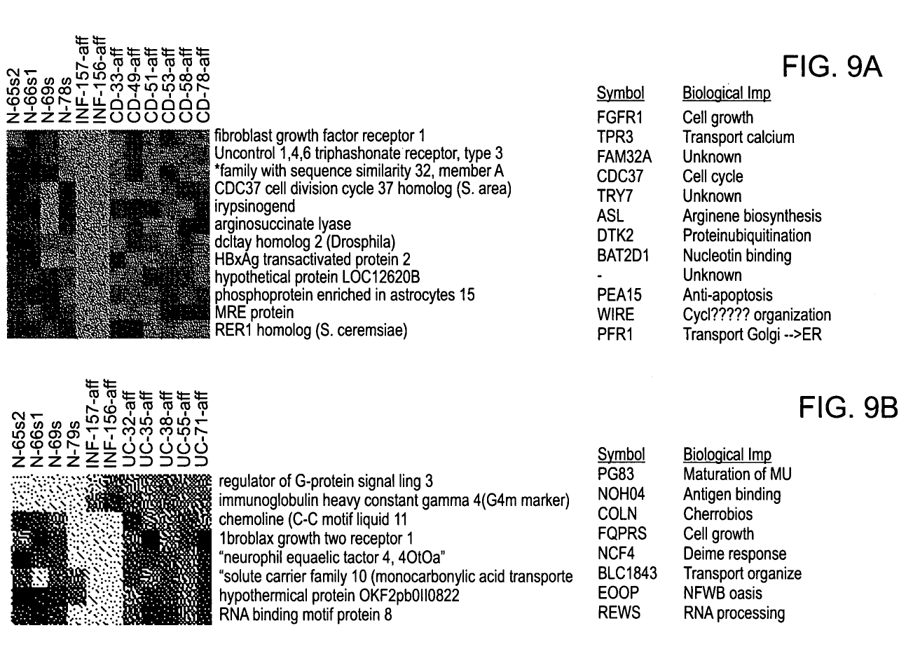

The IBD gene expression patterns were compared with bacterial infectious colitis, as another non-IBD inflammatory disease type, to identify gene expression changes in IBD that may reflect disease-related events. Genes were sought that displayed differential expression between the CD or UC affected samples and bacterial colitis, but were unchanged when comparing bacterial colitis to healthy control. 12 such genes were found to be up-regulated in CD affected samples, as compared to bacterial colitis (FIG. 9A), and 8 genes that are over-expressed in UC affected compared to bacterial colitis and healthy control (FIG. 9B). Except fibroblast growth factor receptor 1 (FGFR1) and Chemokine (C-C motif) ligand 11 (CCL11), the majority of the genes identified here are novel findings in terms of differential expression in IBD. These genes may be useful in discriminating IBD from bacterial infection-related temporary colitis.

### Genes Differentially Expressed in Histologically Unaffected IBD Biopsies Compared to Healthy Control

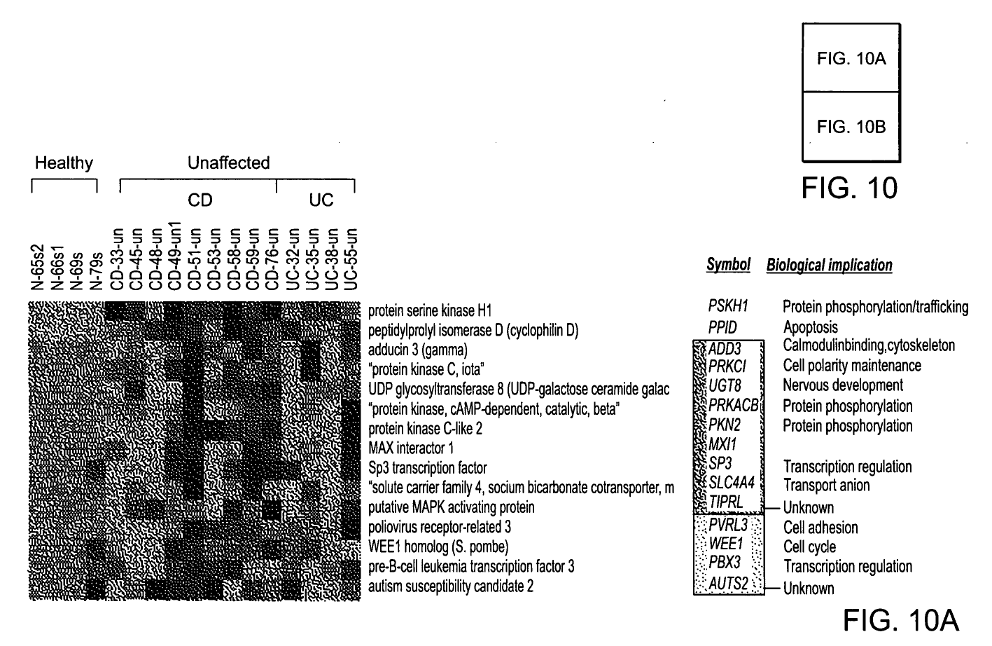

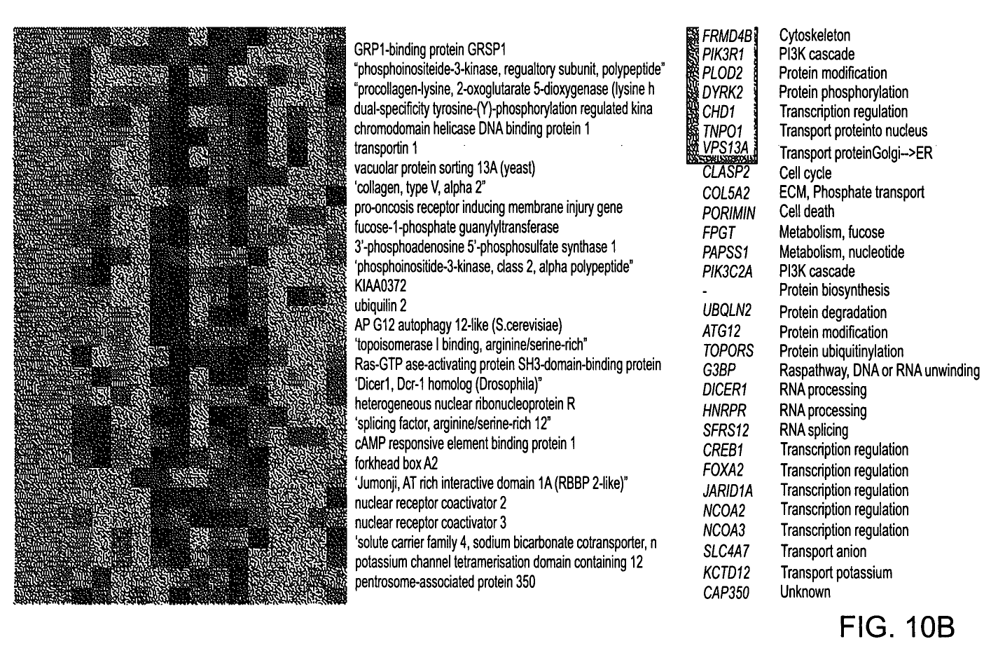

Alterations in gene expression in histologically unaffected IBD biopsies may indicate early pathogenic events before the onset of secondary inflammation. To test this hypothesis, we included colonic biopsies without histological evidence for inflammation (unaffected) from 9 CD and 4 UC patients in this study. A CD case (CD-48) with disease limited to the ileum was included as a true “unaffected” control. At this time, the SAM analysis detected no significant difference in gene expression profiles between the nine CD and the four UC unaffected samples, based on a highly stringent criteria of: False Discovery Rate ≦0.00001%, fold change >2, and Log₂ mean expression index >6.64. We expect that, as sample size increase, distinctive expression patterns between CD and UC unaffected biopsies may be identified. Upon comparing all 13 profiles of CD and UC unaffected samples to the 4 healthy controls, we found that two genes were up regulated and 42 were down regulated in IBD unaffected samples (FIG. 10). The two up-regulated genes, PSKH1 (protein serine kinase H1) and PPID (peptidylprolyl isomerase D), were also up regulated in the affected biopsy tissues of CD and UC patients. Moreover, about half of the genes down-regulated in the unaffected IBD biopsies were also down regulated in IBD affected tissues (Table 18 and supplementary Table 17). The majority of these down-regulated genes function in transcription regulation (9 genes), protein modification and metabolism (8 genes), and transporting anions or proteins (5 genes).

Global gene expression patterns were obtained from single endoscopic pinch biopsies that were reproducible and representative of the local diseased area. We used unsupervised multidimensional scaling (MDS) on our IBD and healthy gene expression profiles to develop a systematic approach towards molecular classification of disease subtypes. A separation was observed of IBD from controls. While most CD cases were closely associated, two other CD cases localized with UC samples, underscoring the heterogeneous nature of IBD. The two CD patients have disease with involvement of the rectum, which are different from other CD patients with rectal sparing. These UC-like CD cases are often ANCA-positive.²⁴ The gene expression differences between these two subtypes of CD underscore the distinctive natures of Crohn's proctitis and others. Thus, with increasing sample size, unsupervised clustering may define stable, meaningful subgroups of CD and UC for further elucidation of differential gene signature.

The unsupervised MDS was able to distinguish IBD from acute bacterial colitis and healthy control samples. Recently, Burczynski, et al²⁵ attempted to classify CD and UC based on gene expression profiles of peripheral blood mononuclear cells of IBD patients. In that study supervised analysis, with pre-defined subgroups, was used first to identify a set of genes, followed by testing their accuracy in distinguishing UC and CD patient samples²⁵. In contrast, the entire data set was analyzed by unsupervised multidimensional scaling. This strategy allowed for an unbiased clustering of samples based on the original data, and the detection of sub-groups within each disease type.

Upon comparing our current biopsy study with previous gene expression studies of resected IBD tissues,¹²⁻¹⁶ it was noted that considerable overlaps in the differential expression patterns. These include expression of IL8, LCN2, NOS2A, REG1A, CXCL1, for example. Profiling of endoscopic biopsy tissues has identified several novel gene expression changes. Key gene expression differences in CD and UC speak of fundamentally different biological processes contributing to their pathogenesis. The genes over-expressed in CD are overwhelmingly those of acute phase, macrophage activation and antigen processing or presentation (Table 16). The proteasome is a multi-protein complex that degrades cellular or foreign protein. The peptides generated by immunoproteasome for MHC class I antigen presentation are translocated into the endoplasmic reticulum by TAP (transporter associated with antigen processing)²⁶. Over expression of proteasome subunit genes (PSME2, PSMB8, PSMB9, PSMB10), and TAP1 in CD affected tissues indicate that the processing pathway of class I MHC peptides is active in CD. Identification of mechanism of such up-regulation and the substrates of these immunoproteasomes may be helpful in understanding the pathophysiology of Crohn's disease.

The UC gene expression pattern suggests disruption of epithelial homeostasis. It was observed that a number of genes functioning in biosynthesis, metabolism, and transport are remarkably down regulated in UC. While the causes for such down regulations are unknown at this time, we speculate that functional loss of specific transcription factors and master regulators may be one reason. Nine (CCNT2, CHD1, HNF4G, KLF5, MLLT2, MYB, NR3C2, SATB2, ZNF217) of 64 genes that are down regulated in UC, are transcription regulators. Mucosal damage and loss of epithelia in chronic UC may be another factor. However, considering that many of the genes are also down regulated in the unaffected samples, we favor the idea that there may be a few key regulators that are affected in UC.

Genes that are consistently differentially expressed in both IBD affected and unaffected biopsies, such as PSKH1 and PPID, may represent early pathogenic changes in IBD. However, much more works is needed to characterize their roles in the development of IBD. PSKH1 plays a role in intracellular protein trafficking and Golgi apparatus maintenance²⁷. Its over expression in the IBD samples may reflect increased synthesis of immunoglobulins and other proteins. PPID encodes for peptidylprolyl isomerase D or cyclophilin D. This protein is a component of mitochondrial permeability transition pore which mediates cytochrome c release leading to apoptosis²⁸. The immunosuppressant cyclosporin A, used to treat severe IBD, particularly corticosteroid-refractory ulcerative colitis,²⁹ can bind the PPID protein³⁰ and reduce mitochondrial permeability and cytochrome c release. Thus, the underlying mechanism of the therapeutic effects of cyclosporin A may be mediated by binding to PPID.

The gene expression profiles have identified several candidate genes within these areas. These include apoptosis-regulating CASP10 at 2q33-34, and antigen-presenting gene PSME2 at 14q11.2 (locus IBD4) from CD profiles, as well as immune response gene IFI30 (19p13.1, IBD6) and Notch-signaling NOTCH3 (19p13.2-p13.1, IBD6) from the UC profiles. With respect to the IBD3 locus at 6p21,³⁶ HLA-DMA, HLA-DRB1, TAP1, UBD and PSMB8 at 6p21.3, are particularly intriguing.
1. Fiocchi C. Inflammatory bowel disease: etiology and pathogenesis. Gastroenterology 1998; 115:182-205.
2. Podolsky D K. Inflammatory bowel disease. N Engl J Med 2002; 347:417-29.
3. Sartor R B. Current concepts of the etiology and pathogenesis of ulcerative colitis and Crohn's disease. Gastroenterol Clin North Am 1995; 24:475-507.
4. Blumberg R S, Strober W. Prospects for research in inflammatory bowel disease. Jama 2001; 285:643-7.
5. Marion J, Rubin P, D. P. Differential diagnosis of chronic ulcerative colitis and Crohn's disease. W. B. Saunders Company, 2000.
6. Sands B E. From symptom to diagnosis: clinical distinctions among various forms of intestinal inflammation. Gastroenterology 2004; 126:1518-32.
7. Golub T R, Slonim D K, Tamayo P, Huard C, Gaasenbeek M, Mesirov J P, Coller H, Loh M L, Downing J R, Caligiuri M A, Bloomfield C D, Lander E S. Molecular classification of cancer: class discovery and class prediction by gene expression monitoring. Science 1999; 286:531-7.
8. Bittner M, Meltzer P, Chen Y, Jiang Y, Seftor E, Hendrix M, Radmacher M, Simon R, Yakhini Z, Ben-Dor A, Sampas N, Dougherty E, Wang E, Marincola F, Gooden C, Lueders J, Glatfelter A, Pollock P, Carpten J, Gillanders E, Leja D, Dietrich K, Beaudry C, Berens M, Alberts D, Sondak V. Molecular classification of cutaneous malignant melanoma by gene expression profiling. Nature 2000; 406:536-40.
9. Hedenfalk I, Duggan D, Chen Y, Radmacher M, Bittner M, Simon R, Meltzer P, Gusterson B, Esteller M, Kallioniemi O P, Wilfond B, Borg A, Trent J. Gene-expression profiles in hereditary breast cancer. N Engl J Med 2001; 344:539-48.
10. Yeoh E J, Ross M E, Shurtleff S A, Williams W K, Patel D, Mahfouz R, Behm F G, Raimondi S C, Relling M V, Patel A, Cheng C, Campana D, Wilkins D, Zhou X, Li J, Liu H, Pui C H, Evans W E, Naeve C, Wong L, Downing J R. Classification, subtype discovery, and prediction of outcome in pediatric acute lymphoblastic leukemia by gene expression profiling. Cancer Cell 2002; 1:133-43.
11. Fuller G N, Hess K R, Rhee C H, Yung W K, Sawaya R A, Bruner J M, Zhang W. Molecular classification of human diffuse gliomas by multidimensional scaling analysis of gene expression profiles parallels morphology-based classification, correlates with survival, and reveals clinically-relevant novel glioma subsets. Brain Pathol 2002; 12:108-16.
12. Lawrance I C, Fiocchi C, Chakravarti S. Ulcerative colitis and Crohn's disease: distinctive gene expression profiles and novel susceptibility candidate genes. Hum Mol Genet 2001; 10:445-56.
13. Dieckgraefe B K, Stenson W F, Korzenik J R, Swanson P E, Harrington C A. Analysis of mucosal gene expression in inflammatory bowel disease by parallel oligonucleotide arrays. Physiol Genomics 2000; 4:1-11.
14. Uthoff S M, Eichenberger M R, Lewis R K, Fox M P, Hamilton C J, McAuliffe T L, Grimes H L, Galandiuk S. Identification of candidate genes in ulcerative colitis and Crohn's disease using cDNA array technology. Int J Oncol 2001; 19:803-10.
15. Dooley T P, Curto E V, Reddy S P, Davis R L, Lambert G W, Wilborn T W, Elson C O. Regulation of gene expression in inflammatory bowel disease and correlation with IBD drugs: screening by DNA microarrays. Inflamm Bowel Dis 2004; 10:1-14.
16. Langmann T, Moehle C, Mauerer R, Scharl M, Liebisch G, Zahn A, Stremmel W, Schmitz G. Loss of detoxification in inflammatory bowel disease: dysregulation of pregnane X receptor target genes. Gastroenterology 2004; 127:26-40.
17. Hommes D W, van Deventer S J. Endoscopy in inflammatory bowel diseases. Gastroenterology 2004; 126:1561-73.
18. Costello C M, Mah N, Hasler R, Rosenstiel P, Waetzig G H, Hahn A, Lu T, Gurbuz Y, Nikolaus S, Albrecht M, Hampe J, Lucius R, Kloppel G, Eickhoff H, Lehrach H, Lengauer T, Schreiber S. Dissection of the inflammatory bowel disease transcriptome using genome-wide cDNA microarrays. PLoS Med 2005; 2:e199.
19. Okahara S, Arimura Y, Yabana T, Kobayashi K, Gotoh A, Motoya S, Imamura A, Endo T, Imai K. Inflammatory gene signature in ulcerative colitis with cDNA macroarray analysis. Aliment Pharmacol Ther 2005; 21:1091-7.
20. Colombel J F, Grandbastien B, Gower-Rousseau C, Plegat S, Evrard J P, Dupas J L, Gendre J P, Modigliani R, Belaiche J, Hostein J, Hugot J P, van Kruiningen H, Cortot A. Clinical characteristics of Crohn's disease in 72 families. Gastroenterology 1996; 111:604-7.
21. Li C, Wong W H. Model-based analysis of oligonucleotide arrays: expression index computation and outlier detection. Proc Natl Acad Sci USA 2001; 98:31-6.
22. Torgerson W S. Theory and Methods of Scaling. Wiley, 1958.
23. Tusher V G, Tibshirani R, Chu G. Significance analysis of microarrays applied to the ionizing radiation response. Proc Natl Acad Sci USA 2001; 98:5116-21.
24. Vasiliauskas E A, Kam L Y, Karp L C, Gaiennie J, Yang H, Targan S R. Marker antibody expression stratifies Crohn's disease into immunologically homogeneous subgroups with distinct clinical characteristics. Gut 2000; 47:487-96.
25. Burczynski M E, Peterson R L, Twine N C, Zuberek K A, Brodeur B J, Casciotti L, Maganti V, Reddy P S, Strahs A, Immermann F, Spinelli W, Schwertschlag U, Slager A M, Cotreau M M, Domer A J. Molecular classification of Crohn's disease and ulcerative colitis patients using transcriptional profiles in peripheral blood mononuclear cells. J Mol Diagn 2006; 8:51-61.
26. Begley G S, Horvath A R, Taylor J C, Higgins C F. Cytoplasmic domains of the transporter associated with antigen processing and P-glycoprotein interact with subunits of the proteasome. Mol Immunol 2005; 42:137-41.
27. Brede G, Solheim J, Stang E, Prydz H. Mutants of the protein serine kinase PSKH1 disassemble the Golgi apparatus. Exp Cell Res 2003; 291:299-312.
28. Machida K, Osada H. Molecular interaction between cyclophilin D and adenine nucleotide translocase in cytochrome c release: does it determine whether cytochrome c release is dependent on permeability transition or not? Ann N Y Acad Sci 2003; 1010:182-5.
29. Pham C Q, Efros C B, Berardi R R. Cyclosporine for severe ulcerative colitis. Ann Pharmacother 2006; 40:96-101.
30. Hoffmann K, Kakalis L T, Anderson K S, Armitage I M, Handschumacher R E. Expression of human cyclophilin-40 and the effect of the His141-->Trp mutation on catalysis and cyclosporin A binding. Eur J Biochem 1995; 229:188-93.
31. Morley M, Molony C M, Weber T M, Devlin J L, Ewens K G, Spielman R S, Cheung V G. Genetic analysis of genome-wide variation in human gene expression. Nature 2004; 430:743-7.
32. Satsangi J, Parkes M, Louis E, Hashimoto L, Kato N, Welsh K, Terwilliger J D, Lathrop G M, Bell J I, Jewell D P. Two stage genome-wide search in inflammatory bowel disease provides evidence for susceptibility loci on chromosomes 3, 7 and 12. Nat Genet 1996; 14:199-202.
33. Hugot J P, Thomas G. Genome-wide scanning in inflammatory bowel diseases. Dig Dis 1998; 16:364-9.
34. Cho J H, Nicolae D L, Gold L H, Fields C T, LaBuda M C, Rohal P M, Pickles M R, Qin L, Fu Y, Mann J S, Kirschner B S, Jabs E W, Weber J, Hanauer S B, Bayless T M, Brant S R. Identification of novel susceptibility loci for inflammatory bowel disease on chromosomes 1p, 3q, and 4q: evidence for epistasis between 1p and IBD1. Proc Natl Acad Sci USA 1998; 95:7502-7.
35. Brant S R, Shugart Y Y. Inflammatory bowel disease gene hunting by linkage analysis: rationale, methodology, and present status of the field. Inflamm Bowel Dis 2004; 10:300-11.
36. Hampe J, Shaw S H, Saiz R, Leysens N, Lantermann A, Mascheretti S, Lynch N J, MacPherson A J, Bridger S, van Deventer S, Stokkers P, Morin P, Mirza M M, Forbes A, Lennard-Jones J E, Mathew C G, Curran M E, Schreiber S. Linkage of inflammatory bowel disease to human chromosome 6p. Am J Hum Genet 1999; 65:1647-55.

### Example 3

### Patient Selection and Endoscopic Pinch Biopsy Acquisition

The study includes patients and control individuals recruited at the Meyerhoff IBD Center from 2001-2003 (Table. 19). Diagnosis of patients was based on primary endoscopic, pathologic and radiology reports using standard diagnostic criteria. ^{10, 15} Patients undergoing colonoscopy included CD and UC patients, non-IBD colitis, and non-IBD healthy controls. All healthy controls were negative for colorectal cancer on screening. All patients received Golytely® colonic preparation.

TABLE 19
Sample information

| | | | Disease | | | | | | | |
|---|---|---|---|---|---|---|---|---|---|---|
| | Age | | | Duration | | Endoscopy | | Histology | | |
| Sample¹ | (years) | Sex | Location | (years) | | Site² | definition | Inflammation³ | Fibrosis⁴ | Medication⁵ |
| CD33aff | 24 | M | colonic | 4 | | Sigmoid | affected | ++ | + | a, c, d |
| CD33un | | | | | | T. colon | unaffected | − | − | |
| CD45un2 | 37 | F | colonic | 12 | | Cecum | affected | − | + | a, b, d |
| CD45un | | | | | | A. colon | unaffected | − | − | |
| CD48un | 44 | M | ileal | 20 | | T. colon | unaffected | − | − | a, b |
| CD48un2 | | | | | | T. colon | unaffected | − | − | |
| CD49aff | 21 | F | ileocolonic | 3 | | Cecum | affected | + | + | a, b, c, d |
| CD49un | | | | | | Rectum | unaffected | − | + | |
| CD51aff | 39 | M | ileocolonic | 15 | | Sigmoid | affected | ++ | + | a, c |
| CD51un | | | | | | SF. colon | unaffected | − | − | |
| CD53aff | 55 | M | colonic | 15 | | Rectum | affected | +++ | ++ | c, d |
| CD53un | | | | | | T. colon | unaffected | − | − | |
| CD58aff | 51 | F | colonic | 10 | | SF. colon | affected | + | + | a, c |
| CD58un | | | | | | D. colon | unaffected | − | − | |
| CD59un2 | 32 | M | ileocolonic | 15 | | T. colon | affected | − | − | a |
| CD59un | | | | | | D. colon | unaffected | − | − | |
| CD76aff1 | 76 | M | colonic | 2 | | Sigmoid | affected | ++ | ++ | a, b |
| CD76aff2 | | | | | | Sigmoid | affected | ++ | ++ | |
| CD76un | | | | | | Rectum | unaffected | − | − | |
| Mean | 42.1 | | | 13 | | | | | | |
| Range | 21-76 | | | 1-30 | | | | | | |
| UC32aff | 82 | F | Pancolitis | 15 | | Rectum | affected | + | ++ | a |
| UC32un | | | | | | R. colon | unaffected | − | + | |
| UC35aff | 40 | F | Distal | 24 | | Rectum | affected | ++ | ++ | c, d |
| | | For | | | | | | | | |
| | | Peer | | | | | | | | |
| | | Review | | | | | | | | |
| UC35un | | | | | | A. colon | unaffected | − | − | |
| UC38aff | 60 | M | Distal | 12 | | Sigmoid | affected | + | − | a, c |
| UC38un | | | | | | A. colon | unaffected | − | − | |
| UC55aff | 64 | F | Distal | 46 | | Rectum | affected | ++ | ++ | a, d |
| UC55un | | | | | | HF. colon | unaffected | − | − | |
| UC71aff | 54 | M | Pancolitis | 3 | y | Sigmoid | affected | + | ++ | a |
| Mean | 60 | | | 20 | | | | | | |
| Range | 40-82 | | | 3-46 | | | | | | |
| INF156 | 33 | F | Distal | 1/12 | | Sigmoid | affected | ++ | − | b |
| INF157 | 72 | F | colon | 2/12 | | Rectum | affected | +++ | − | b |
| IC44aff | 45 | M | colon | 4 | | Sigmoid | affected | + | − | a, b |
| IC44un | | | | | | D. colon | affected | − | − | |
| Mean | 50 | | | | | | | | | |
| Range | 33-72 | | | <4 | | | | | | |
| N65 | 22 | F | | | | Sigmoid | normal | −− | | |
| N66 | 64 | M | | | | Sigmoid | normal | | | |
| N69 | 65 | F | | | | Sigmoid | normal | −− | | |
| N79 | 57 | F | | | | Sigmoid | normal | −− | | |
| Mean | 52 | | | | | | | | | |
| Range | 22-64 | | | | | | | | | |

Endoscopic pinch biopsies for affected and unaffected areas of IBD cases were obtained from various regions of the colon as listed in Table 19. The samples were tentatively labeled as “affected” when the biopsy was taken from an area appearing grossly affected, and as “unaffected” when taken from an area appearing disease-free, and located 10 cm away from diseased areas. Classification of “affected” or “unaffected” samples for the microarray study was confirmed in a blinded manner by histological examination of adjacent biopsy samples for the presence or absence of acute or chronic inflammation by a gastroenterology pathologist (A.M.). CD-45, -48 and -59 initially included as affected biopsy samples in the microarray study were subsequently labeled as “unaffected” (un2) because adjacent biopsies did not demonstrate active inflammation.

### RNA Isolation and Microarray

Each biopsy, approximately 2×2×3 mm³ and weighing 2-7 mg (mean=4.7 mg, n=6 biopsies), produced ˜5 μg total RNA (TRIzol Reagent, Invitrogen), yielding 15 μg of biotin-labeled cRNA (https://www.affymetrix.com/support/technical/manual/). The biotinylated cRNA (10 μg per array) was hybridized to high-density oligonucleotide GeneChip Human Genome U95Av2 arrays (12,625 probe sets for 9662 unique transcripts, UniGene database, Build #95, Affymetrix). The arrays were washed and stained (R-Phycoerythrin Streptavidin) in a GeneChip Fluidics Station 400. Images captured in a HP Gene Array Scanner were analyzed first by Microarray Suite 5.0 software (Affymetrix). Each transcript received a “present” or “absent” designation based on whether the gene transcript was detected in the sample. The background intensities were low (40±0.6 to 52±1.0 arbitrary units), with ˜48.4% to 56.9% of all 12,625 probe sets marked as “present” in the biopsy samples, consistent with our previous study of whole colon tissue resections.⁵ The complete dataset is available at the NCBI Gene Expression Omnibus (which may be found on the world wide web at www.ncbi.nlm.nih.gov/geo), GSE 6731.

### Microarray Data Analysis

The DNA-Chip Analyzer (dChip) software¹⁶ was used to normalize the data from the image files for array-to-array comparisons. We used (1) classical Multidimensional Scaling (MDS),¹⁷ which provides a two-dimensional rendering of the data to show the correlation of the gene expression among various samples,¹⁸ such that, samples with similar expression profiles lie closer to each other, and (2) Significance Analysis of Microarrays (SAM) software,¹⁹ to select biologically significant changes in gene expression between groups. The criteria selected for SAM analysis are, a median false discovery rate (FDR) ≦0.00001%, fold change >2, and a Log₂ mean expression index >6.64.

### Quantitative RT-PCR

Expressions of genes (PSME2, PSMB8, ADM, STAT3, CXCL1, DMBT1 and GAPDH) were quantified by real-time RT-PCR using QuantiTect SYBR Green PCR Kit (Qiagen Inc., Valencia, USA) according to manufacturer's instruction. The specific primers for genes selected are as follows: GAPDH sense primer, 5′-GTC-TCC-TCT-GAC-TTC-AAC-A-3′; GAPDH antisense primer, 5′-CAG-GAA-ATG-AGC-TTG-ACA-AA-3′; PSME2 sense primer, 5′-ACC-TGA-TCC-CCA-AGA-TTG-AA-3′; PSME2 antisense primer, 5′-TGG-AAA-TGG-TTG-TCT-GGA-AAG-3′; PSMB8 sense primer, 5′-TAA-GTC-CAA-GGA-GAA-GAA-GAG-3′; PSMB8 antisense primer, 5′-CAA-ATA-GAG-AAC-ACG-CAG-AAG-A-3′; ADM sense primer, 5′-CAG-CGA-GTG-TAA-AGT-TG-3′; ADM anti sense primer, 5′-GAG-TCG-GTG-TTT-CCT-TCT-TC-3′; DMBT1 sense primer, 5′-TGC-TGT-ACT-GAC-CTT-GTT-TG-3′; DMBT1 antisense primer, 5′-GGG-TCC-GTA-GGT-GTC-ATC-3′; CXCL1 sense primer, 5′-CCA-AAG-TGT-GAA-CGT-GAA-G-3′; CXCL1 antisense primer, 5′-TGG-GGG-ATG-CAG-GAT-TGA-3′; STAT3 sense primer, 5′-TTT-TAC-CAA-GCC-CCA-AT-3′; STAT3 antisense primer, 5′-TGC-TCG-ATG-CTC-AGT-CCT-3′. The reactions were performed on an ABI PRISM 7900HT system (Applied Biosystems, Foster City, Calif., USA) as follows: initial step at 95° C. for 15 min and 40 cycles at 95° C. for 15 sec, 55° C. for 30 sec and 72° C. for 30 sec followed by a step of ramp temperature from 60° C. to 95° C. at the rate of 2%. The relative expression value is defined as 2^{ΔC}^{_{T}}, where ΔC_{T}=(C_{T} of GAPDH−C_{T} of gene X)−(C_{T} of GAPDH no template control−C_{T} of gene X no template control).

### Immunohistochemistry

Immunohistochemistry was performed on paraffin-embedded sections of colonoscopic pinch biopsies from 2 CD and 2 healthy controls. An ABC-staining kit with the rabbit anti-human TAP1 antibody (Santa Cruz Biotechnology, Santa Cruz, USA) was used. The slides were counter-stained with Hematoxylin Gill No. 2 (Sigma).

### Statistical Analysis

The quantitative RT-PCR results were presented as Box-Whisker charts using Microcal Origin v6.0 (Microcal Software, Inc. Northampton, Mass.). The box represents 25th and 75th percentiles of the data set, with the 50th percentile shown as a line in the box, and the data range (1 to 99 percentile) is indicated by the whiskers. Statistical analyses were performed with one-way unpaired Student's t test for comparing pairs of groups and P<0.05 was considered statistically significant. To test whether distribution of samples in the MDS plot was dependent on gene expression or location of biopsy samples in the colon, a non-parametric test of statistical significance was performed using the Chi square test.

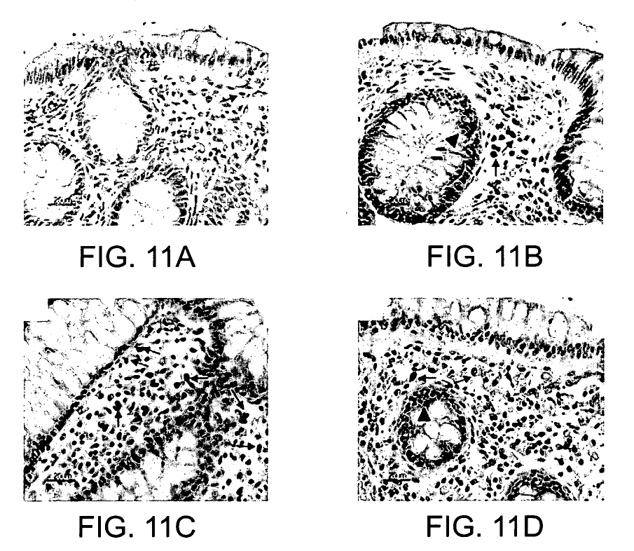

A total of nine CD, six UC, one clinically reclassified as indeterminate colitis (IC), two infectious colitis (INF) and four healthy control individuals were recruited for this study. For each individual included in the study information on age, gender, medications, disease location, duration, biopsy site and presence of inflammation and fibrosis as assessed by histology are provided in Table 19. The IBD biopsy samples were finally designated as “affected” or “unaffected” based on the histopathology of adjacent biopsies (FIG. 11). Control and unaffected biopsies essentially displayed normal colonic architecture. In contrast, biopsies marked as “affected” manifested variable degrees of acute or chronic colitis, including one or more of the following histologic features: cryptitis, with or without accompanying crypt abscesses, crypt distortion, lamina propria fibrosis, crypt dropout, basal lymphoplasmacytosis, and Paneth cell metaplasia. None of the biopsies indicated evidence of colitis-associated epithelial dysplasia or neoplasia (data not shown).

### Reproducibility of Gene Expression Patterns

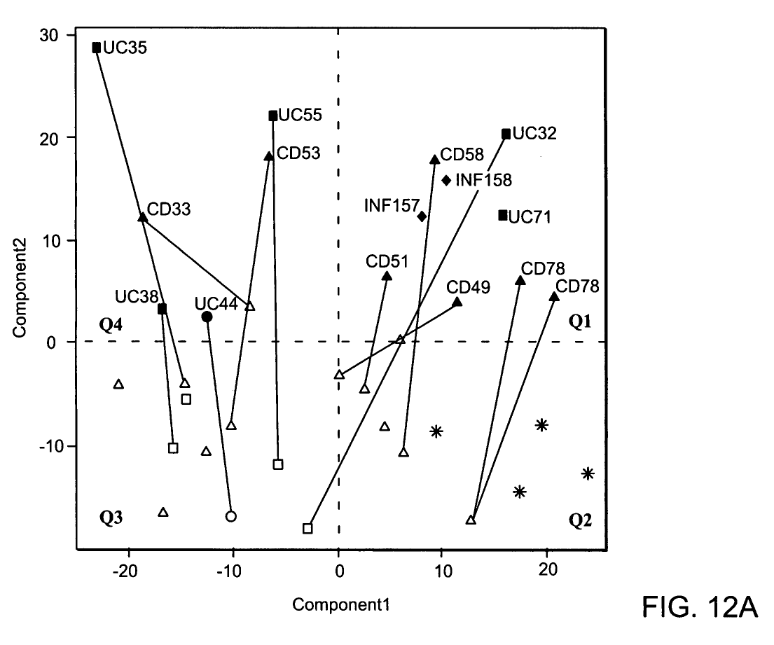

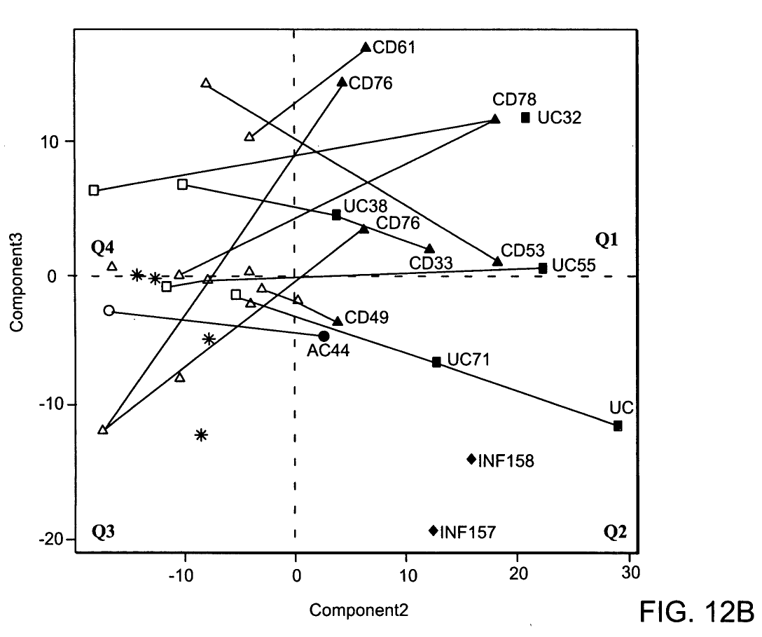

To evaluate if one pinch biopsy was representative of disease in that colonic segment, and to estimate variations in gene expression that could arise from separate samplings, expression patterns of 2 biopsies, 10 cm apart, from one affected area of a CD patient (CD76aff1 and CD76aff2) were analyzed. We chose Crohn's disease because of its characteristic focal areas of tissue damage interspersed with relatively normal appearing tissues. Between CD76aff1 and CD76aff2, only ten of the 3384 “present” genes classified as present by the Microarray Suite software (Methods), demonstrated a greater than two fold difference in expression, with an error of 0.29% in independent gene expression measurements (FIG. 12). Thus, the gene expression pattern from a single endoscopic pinch biopsy was considered a highly reproducible reflection of gene expression in a given tissue designation.

### Sample Classification by Multidimensional Scaling

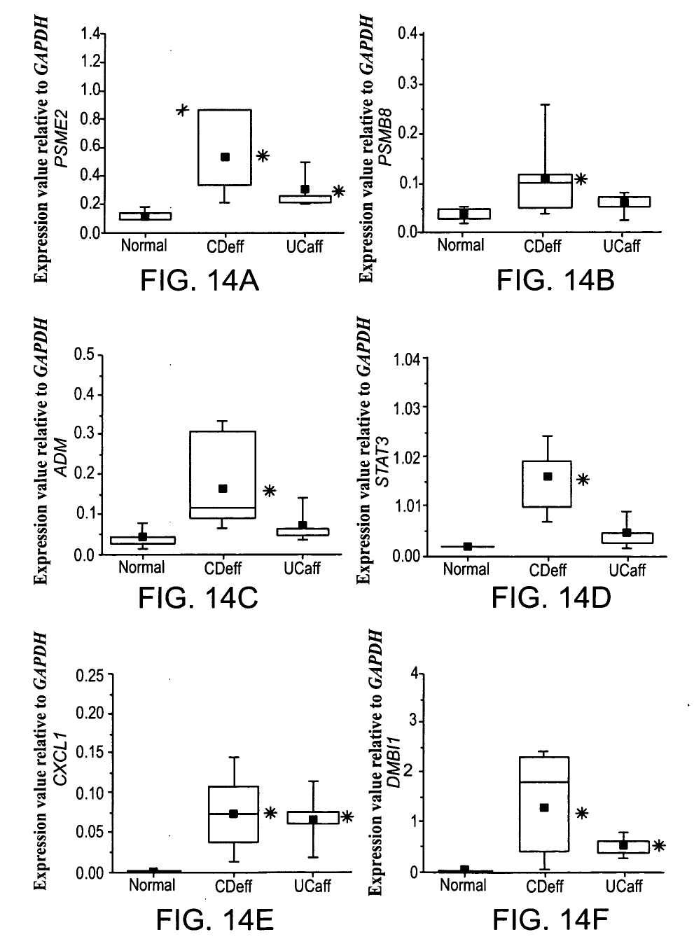

A clustering method of multidimensional scaling (MDS) was applied to the entire microarray data set (36 profiles). The purpose of this exercise was to determine if CD, UC and non-IBD colitis display consistent gene expression differences due to inherent pathogenic differences, such that samples can be separated based on their expression patterns alone. The MDS analysis employs an unsupervised (no pre-defined groups) method such that samples are placed in a two-dimensional space, in which the distance between samples reflects the degree of correlation and samples sharing gene expression similarities appear closer together in such a plot. The samples showed some grouping and separation along the first three axes/components. A graphical representation of component 1 versus component 2 resulted in separation of samples along component 2, with affected tissues placed above the horizontal median in the two upper quadrants (Q1 and Q4) and unaffected tissues and healthy controls in the lower two quadrants (Q2 and Q3) (FIG. 14A). Thus component 2 is the major axis that appeared to separate disease from unaffected and normal samples. There was some clustering of CD and separation from UC along component 1 axis. Four CD cases with active disease were clustered together in Q1 (CD 51, CD49, CD58 and CD76); clinically these have ileocolonic and colonic disease involvement with rectal sparing. Two other CD cases, CD 33 and CD53, positioned with UC samples in Q4, presented rectal disease, with high histopathologic inflammation scores. CD33 had disease extending from the rectum to the splenic flexure, without more proximal involvement resembling UC; CD53 had significant disease in the distal 20 cm of the colon, and inactive disease in the ascending and descending colon, also resembling UC. The two INF samples appeared in Q1, separated from normal controls and unaffected IBD samples. Three of the UC affected samples were positioned in Q4, while UC32aff and UC71aff, with pancolitis, appeared with CD affected samples in Q1. The normal controls were clearly separated from affected colitis samples, and placed in Q2. Unaffected UC samples were separated from the normal controls and placed in Q3 whereas about half of the CD unaffected samples were placed in Q2 with the normal controls and the rest with UC unaffected in Q3. The component 2 versus component 3 MDS plot was far more effective in separating infectious colitis from IBD (FIG. 14B); in this plot the INF samples were distanced from all IBD except two UC affected cases (UC71 and UC35). Overall, MDS analysis allowed certain clustering of samples along disease types.

One concern was that site of origin for the biopsy samples could be affecting the MDS distribution. Therefore we examined the correlation between distribution of samples in the MDS plots and biopsy site or disease type. The normal controls came from the sigmoid colon and appeared below the horizontal axis in Q2 of the first MDS plot (FIG. 14C). Several affected samples were also from the sigmoid colon, yet these did not appear with the control sigmoid colon samples. Rather, affected sigmoid colon samples appeared above the horizontal axis in Q1 and Q4, with other disease biopsy samples. A chi square test (Table. 20A) of our hypothesis, that disease samples (CDaff, UCaff and INF) have a non-random distribution along component 2, above the horizontal axis in Q1 and Q4, while unaffected and normal samples below the horizontal axis, in Q2 and Q3, yielded a highly significant (p<0.001) chi square value of 27.708 with one degree of freedom. We next tested if biopsy site affected distribution of samples in the MDS plot. The distribution of biopsy samples, C1 (rectum+sigmoid colon), C2 (descending colon+splenic flexure), C3 (transverse colon) and C4 (hepatic flexure, ascending colon+cecum), was tested in Q1Q4 above the horizontal axis, or Q2Q3 below the horizontal axis. A chi square value of 7.03 at three degrees of freedom, and p≦0.1 indicated that their distribution in the MDS plot occurred at random and not correlated to biopsy location (Table. 20B). Moreover, a recent study reported that systematic comparison of gene expression from biopsies taken from different regions within the large intestine showed no significant difference¹⁴. A mouse study has also shown that within the large intestine gene expression patterns from different areas were similar, while expression patterns tended to vary between the stomach, small and large intestine.²⁰ We further found that the position of samples in the MDS map was independent of patients' age, gender, disease duration or medication.

TABLE 20A
Distribution of diseased/non-diseased samples in MDS

| Sample | Q1Q4 | Q2Q3 | Total |
|---|---|---|---|
| D | 14 | 0 | 14 |
| N | 2 | 19 | 21 |
| Total | 16 | 19 | 35 |

D: diseased,
N: non-diseased or healthy control or unaffected.
Q1-Q4: quadrants from FIG. 14A.
Degrees of freedom = 1, Chi square = 27.708, with P_0.001, the distribution is significant.

TABLE 20B
Distribution of biopsy location in MDS

| Sample | Q1Q4 | Q2Q3 | Total |
|---|---|---|---|
| C1 | 12 | 6 | 18 |
| C2 | 1 | 3 | 4 |
| C3 | 2 | 4 | 6 |
| C4 | 1 | 6 | 7 |
| Total | 16 | 19 | 35 |

C1: rectum and sigmoid colon,
C2: descending colon and splenic flexure,
C3: transverse colon and
C4: hepatic flexure, ascending colon and cecum. Degrees of freedom = 3 Chi square = 7.03262, with P_0.1, the distribution is not significant.

### Significance Analysis of Microarrays to Identify Genes Differentially Expressed in IBD Compared to Normal Controls

The MDS analysis indicated that the gene expression patterns in affected biopsy samples from IBD were sufficiently different for these to be separated from normal controls and that, among the affected samples, there was some indication of disease-biased separation of samples. Therefore, we next proceeded to identify genes expression differences that define CD and UC and distinguish these from non-IBD colitis. A Significance Analysis of Microarrays (SAM) was performed on the gene expression data, using a set of stringent criteria (see Methods) to identify differences in CD affected, UC affected and infectious colitis as a non-IBD inflammatory control compared to normal controls. A numeric distribution of differentially expressed genes is shown in Table 21. Up regulated genes included 47 in CD affected, 51 in UC affected and 10 in INF, while 30 genes in CD, 81 in UC and 53 in INF were down regulated (Table. 21). UC and CD share 25 up-regulated and 18 down regulated genes. Of the 10 up regulated genes in INF, 4 were commonly up regulated in UC and CD. There was a greater similarity between UC and INF with respect to down regulated genes with 20 genes commonly down regulated in both.

TABLE 21

| | | |
|---|---|---|
| CD | UC | INF |

| | | | | | | |
|---|---|---|---|---|---|---|
| CD | 47 | 30 | 25 | 18 | 5 | 6 |
| UC | 25 | 18 | 51 | 81 | 4 | 20 |
| INF | 5 | 6 | 4 | 20 | 10 | 53 |

Over expressed genes are in red and under expressed genes in green.

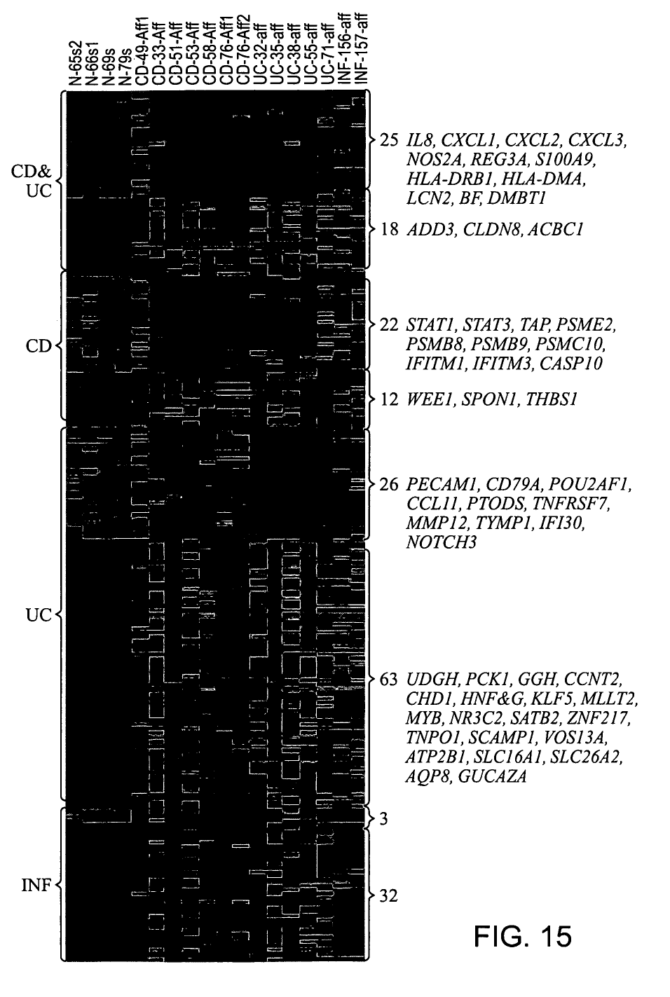

FIG. 15 shows a heat image of all differentially expressed genes in affected IBD samples compared to normal control. Genes preferentially over expressed in CD (Table. 22) included interferon γ inducible genes (IFITM1, IFITM3, STAT1 and STAT3) and those regulating antigen processing and presentation (TAP1, PSME2, PSMB8, PSMB9 and PSMB10). Down regulated genes in CD included WEE1, SPON1 and THBS1 that may be indicative of altered cell-proliferation and cell-ECM adhesion properties. NOS2A, REG3A, IL 8, S100A9, CXCL1, CXCL2 and CXCL3 functioning in inflammatory processes, were up regulated in both CD and UC (Table. 23), while S100A9, CXCL1 and CXCL3 were also elevated in INF. The CXC chemokine ligands regulate chemotaxis and inflammatory cell influx, and their up regulation in infectious colitis suggests that these genes mediate inflammatory events common to most colitis types. Overlapping down regulations in UC and CD (Table. 23) included genes required for maintaining epithelial cellular architecture (adducin3) and epithelial tight junctions (Claudin8), and may reflect pathogenic changes in epithelial integrity secondary to intestinal inflammation. ABCB1, encoding the multidrug resistance p-glycoprotein 170 was down regulated in UC, CD and INF. Genes with biased over expression in UC (Table. 24) included those regulating trans-endothelial migration of platelets and leukocytes (PECAM1), B lymphocyte functions (CD79A, POU2AF1), inflammation mediators that were not detected in either Crohn's or infectious colitis, such as, CCL11, PTGDS, TNFRSF7, and ECM-remodeling genes MMP12 and TIMP1. Genes down-regulated in UC related to biosynthetic and metabolic processes (UGDH, PCK1, GGH), transcription (CCNT2, CHD1, HNF4G, KLF5, MLLT2, MYB, NR3C2, SATB2, ZNF217), protein trafficking (TNPO1, SCAMP1, VPS13A), epithelial electrolyte and water transport (ATP2B1, SLC16A1, SLC26A2, AQP8). A number of genes functioning in transport of electrolyte (SLC26A2, GUCA2A and GUCA2B) and water (AQP8) were also down regulated in infectious colitis.

TABLE 22
Differential gene expression in affected CD compared to healthy control

| | Symbol | Biological impliciation | Cytoband |
|---|---|---|---|
| Up-regulated Gene | | | |
| Adrenomedullin | ADM | Acute-phase response | 11p15.4 |
| Serum amyloid A1 | SAA1 | Acute-phase response | 11p15.1 |
| Serine/cysteine proteinase inhibitor, clade A, 1 | SERPINA1 | Acute-phase response | 14q32.1 |
| Signal transducer and activator of transcription 1 | STAT1 | Acute-phase response | 2q32.2 |
| Signal transducer and activator of transcription 3 | STAT3 | Acute-phase response | 17q21.31 |
| MHC, class II, DR beta 5 | HLA-DRB5 | Antigen presentation | 6p21.3 |
| Transporter 1, ATP-binding cassette, sub-family B | TAP1 | Antigen presentation | 6p21.3 |
| Proteasome activator subunit 2 (PA28 beta) | PSME2 | Antigen presentation | 14q11.2 |
| Proteasome subunit, beta type, 8 | PSMB8 | Antigen presentation | 6p21.3 |
| Proteasome subunit, beta type, 9 | PSMB9 | Antigen presentation | 6p21.3 |
| Proteasome subunit, beta type, 10 | PSMB10 | Antigen presentation | 16q22.1 |
| Interferon, alpha-inducible protein (clone IFI-6- | G1P3 | Immune response | 1p35 |
| 16) | | | |
| Leukocyte Ig-like receptor, subfamily B, member 1 | LILRB1 | Antigen binding | 19q13.4 |
| Interferon induced transmembrane protein 1 (9- | IFITM1 | Macrophage activation | 11p15.5 |
| 27) | | | |
| Interferon induced transmembrane protein 3 (1- | IFITM3 | Macrophage activation | 11p15.5 |
| 8U) | | | |
| Interferon stimulated gene 20 kDa | ISG20 | Macrophage activation | 15q26 |
| Caspase 10 | CASP10 | Apoptosis | 2q33-q34 |
| Mucin 4, tracheobronchial | MUC4 | Cell adhesion | 3q29 |
| Regenerating islet-derived 1 beta | REG1B | Cell proliferation | 2p12 |
| Mucin 1, transmembrane | MUC1 | Cytoskeleton | 1q21 |
| Serine protease inhibitor, Kazal type 4 | SPINK4 | Endopeptidase inhibitor | 9p13.3 |
| Lipin 1 | LPIN1 | Adipocyte | 2p25.1 |
| | | differentiation | |
| Down-regulated | | | |
| Down syndrome critical region gene 1-like 1 | DSCR1L1 | Calcium-mediated | 6p21.1-p12.3 |
| | | signaling | |
| Spondin 1, extracellular matrix protein | SPON1 | Cell adhesion | 11p15.2 |
| Thrombospondin 1 | THBS1 | Cell motility | 15q15 |
| Chemokine (C—X—C motif) ligand 12 | CXCL12 | Chemotaxis | 10q11.1 |
| Stathmin-like 2 | STMN2 | Neuron cell | 8q21.13 |
| | | differentiation | |
| Serine/cysteine proteinase inhibitor, clade B, 7 | SERPINB7 | Proteinase inhibitor | 18q21.33 |
| WEE1 homolog (S. pombe) | WEE1 | Regulation of cell cycle | 11p15.3-p15.1 |
| Myosin, heavy polypeptide 11, smooth muscle | MYH11 | Striated muscle | 16p13.13-P13.12 |
| | | contraction | |
| Chromosome 14 ORF116 (checkpoint | CHES1 | Transcription regulation | 14q24.3-q32.11 |
| suppressor 1) | | | |
| Pre-B-cell leukemia transcription factor 3 | PBX3 | Transcription regulation | 9q33-q34 |
| Autism susceptibility candidate 2 | AUTS2 | Unknown | 7q11.22 |
| Poliovirus receptor-related 3 | PVRL3 | Unknown | 3q13 |

TABLE 23
Gene expression overlaps in CD and UC compared to healthy control

| | | Biological | |
|---|---|---|---|
| | Symbol | implication | Cytoband |
| Up-regulated | | | |
| Ig heavy constant gamma 4 (G4m marker) | IGHG4 | Antigen binding | 14q32.33 |
| MHC, class II, DM alpha | HLA-DMA | Antigen presentation | 6p21.3 |
| MHC, class II, DR beta 1 | HLA- | Antigen presentation | 6p21.3 |
| | DRB1 | | |
| Defensin, alpha 6, Paneth cell-specific | DEFA6 | Antimicrobial | 8pter-p21 |
| Chemokine (C—X—C motif) ligand 1 | CXCL1 | Chemotaxis | 4q21 |
| Chemokine (C—X—C motif) ligand 2 | CXCL2 | Chemotaxis | 4q21 |
| Chemokine (C—X—C motif) ligand 3 | CXCL3 | Chemotaxis | 4q21 |
| Interleukin 8 | IL8 | Chemotaxis | 4q13-q21 |
| B-factor, properdin | BF | Immune response | 6p21.3 |
| Decay accelerating factor for complement | DAF | Immune response | 1q32 |
| Deleted in malignant brain tumors 1 | DMBT1 | Immune response | 10q25.3-q26.1 |
| Lipocalin 2 (oncogene 24p3) | LCN2 | Inflammatory response | 9q34 |
| Nitric oxide synthase 2A (inducible, hepatocytes) | NOS2A | Inflammatory response | 17q11.2-q12 |
| Regenerating islet-derived 3 alpha | REG3A | Inflammatory response | 2p12 |
| S100 calcium binding protein A9 (MRP14) | S100A9 | Inflammatory response | 1q21 |
| Caspase 1 | CASP1 | Apoptosis | 11q23 |
| Peptidylprolyl isomerase D (Cyclophilin D) | PPID | Apoptosis suppressor | 4q31.1 |
| Pim-2 oncogene | PIM2 | Cell proliferation | Xp11.23 |
| Regenerating islet-derived 1 alpha | REG1A | Cell proliferation | 2p12 |
| Tryptophanyl-tRNA synthetase | WARS | Cell proliferation | 14q32.31 |
| | | inhibition | |
| Regulator of G-protein signalling 3 | RGS3 | Inactivation of MAPK | 9q32 |
| Hypothetical protein FLJ12443 | FLJ12443 | Muscle development | 5p15.33 |
| Protein serine kinase H1 | PSKH1 | Protein phosphorylation | 16q22.1 |
| Ubiquitin-conjugating enzyme E2L 6 | UBE2L6 | Ubiquitin cycle | 11q12 |
| PDZK1 interacting protein 1 For Peer Review | PDZK1IP1 | Unknown | 1p33 |
| Down-regulated | | | |
| Adducin 3 (gamma) | ADD3 | Calmodulin binding | 10q24.2-q24.3 |
| Claudin 8 Protein kinase C, iota | CLDN8 | Cell-cell adhesion Cell | 21q22.11 3q26.3 |
| | PRKCI | polarity maintenance | |
| UDP glycosyltransferase 8 | UGT8 | Nervous development | 4q26 |
| BTB (POZ) domain containing 3 | BTBD3 | Protein binding | 20p12.2 |
| Protein kinase C-like 2 | PKN2 | Protein phosphorylation | 1p22.2 |
| Protein kinase, cAMP-dependent, catalytic, beta | PRKACB | Protein phosphorylation | 1p36.1 |
| ATP-binding cassette, sub-family B | ABCB1 | Transporter Transport, | 7q21.1 4q21 |
| (MDR/TAP), 1 Solute carrier family 4, member 4 | SLC4A4 | anion | |
| MAX interactor 1 | MXI1 | Transcription regulation | 10q24-q25 |
| Sp3 transcription factor | SP3 | Transcription regulation | 2q31 |
| Frizzled-related protein | FRZB | Wnt receptor signaling | 2qter |
| Fk506-Binding Protein, Alt. Splice 2 | — | Unknown | — |
| mRNA; cDNA DKFZp586B211 | — | Unknown | — |
| Chromosome 14 open reading frame 11 | C14orf11 | Unknown | 14q13.1 |
| Creatine kinase, brain | CKB | Unknown | 14q132 |
| Transcribed sequences | KIAA1651 | Unknown | — |
| Putative MAPK activating protein | TIPRL | Unknown | 1q23.2 |

TABLE 24
Differential gene expression in affected UC tissues compared to healthy
control

| | Symbol | Biological implication | Cytoband |
|---|---|---|---|
| Up-regulated Gene | | | |
| Coronin, actin binding protein, 1A | CORO1A | Cell motility | 16p11.2 |
| Matrix metalloproteinase 12 | MMP12 | Cell motility | 11q22.3 |
| Platelet/endothelial cell adhesion molecule | PECAM1 | Cell motility | 17q23 |
| (CD31) | | | |
| Talin 1 | TLN1 | Cell motility | 9p13 |
| Tissue inhibitor of metalloproteinase 1 | TIMP1 | Cell motility | Xp11.3-p11.23 |
| Interferon, gamma-inducible protein 30 | IFI30 | Immune response | 19p13.1 |
| POU domain, class 2, associating factor 1 | POU2AF1 | Immune response, | 11q23.1 |
| | | humoral | |
| Clusterin (complement lysis inhibitor, SP-40,40) | CLU | Immune | 8p21-p12 |
| | | response/apoptosis | |
| TNF receptor superfamily, member 7 | TNFRSF7 | Immune | 12p13 |
| | | response/apoptosis | |
| Prostaglandin D2 synthase | PTGDS | Inflammatory response | 9q34.2-q34.3 |
| CD79A antigen (Ig-associated alpha) For Peer | CD79A | Defense response | 19q13.2 |
| Review | | | |
| Defensin, alpha 5, Paneth cell-specific | DEFA5 | Antimicrobial response | 8pter-p21 |
| Ubiquitin D | UBD | Antimicrobial response | 6p21.3 |
| Chemokine (C-C motif) ligand 11 | CCL11 | Chemotaxis, eosinophil | 17q21.1-q21.2 |
| Insulin-like growth factor binding protein 5 | IGFBP5 | Regulation of cell | 2q33-q36 |
| | | growth | |
| Endothelial cell growth factor 1 (platelet-derived) | ECGF1 | Angiogenesis | 22q13 |
| Fascin homolog 1, actin-bundling protein | FSCN1 | Cell proliferation | 7p22 |
| Ataxia telangiectasia mutated | ATM | Apoptosis | 11q22-q23 |
| Notch homolog 3 (Drosophila) | NOTCH3 | Notch signaling | 19p13.2-p13.1 |
| Protease inhibitor 3, skin-derived (SKALP) | PI3 | Endopeptidase inhibitor | 20q12-q13 |
| Nucleoporin 210 | NIP210 | Development | 3p25.2-p25.1 |
| AT rich interactive domain 5A (MRF1-like) | ARID5A | DNA binding | 2q11.2 |
| Pyruvate dehydrogenase kinase, isoenzyme 3 | PDK3 | Protein phosphorylation | Xp22.11 |
| Cathepsin H | CTSH | Proteolysis | 15q24-q25 |
| Lymphocyte cytosolic protein 1 (L-plastin) | LCP1 | Unknown | 13q14.3 |
| Stomatin | STOM | Unknown | 9q34.1 |
| Down-regulated | | | |
| Semaphorin 6A-1 | SEMA6A | Apoptosis | 5q23.1 |
| KIAA0931 protein (PH domain and leucine rich | PHLPPL | Biosynthesis, cAMP | 16q22.2 |
| Repeat protein phosphatase-like) | | | |
| Mitochondrial ribosomal protein S6 | MRPS6 | Biosynthesis, protein | 21q21.3-q22.1 |
| Sterol-C5-desaturase (ERG3 delta-5-desaturase | SC5DL | Biosynthesis, steroid | 11q23.3 |
| Homolog, fungal)-like | | | |
| Related RAS viral (r-ras) oncogene homolog 2 | SCP2 | Biosynthesis, steroid | 11p15.2 |
| UDP-glucose dehydrogenase | UGDH | Biosynthesis | 4p15.1 |
| Calpastatin | CAST | calpain inhibitor | 5q15-q21 |
| | | activity | |
| ADAM-like, decysin 1 | ADAMDEC1 | cell adhesion inhibition | 8p21.2 |
| Dynein, axonemal, heavy polypeptide 9 | DNAH9 | cell motility | 17p12 |
| Ephrin-A1 | EFNA1 | cell-cell signaling | 1q21-q22 |
| Fibroblast growth factor receptor 3 | FGFR3 | JAK-STAT signaling | 4p16.3 |
| Methylmalonyl Coenzyme A mutase | MUT | Metabolism | 6p21 |
| Phosphoenolpyruvate carboxykinase 1 (soluble) | PCK1 | Metabolism, | 20q13.31 |
| | | gluconeogenesis | |
| Gamma-glutamyl hydrolase | GGH | Metabolism, glutamine | 8q12.3 |
| N-acylsphingosine amidohydrolase-like | ASAHL | Metabolism | 4q21.1 |
| Acyl-Coenzyme A dehydrogenase, | ACADM | Metabolism, lipid | 1p31 |
| UDP glycosyltransferase 2 family, B28 | UGT2B28 | Metabolism, lipid | 4q13 |
| Ectonucleoside triphosphate diphosphohydrolase 5 | ENTPD5 | Metabolism, | 14q24 |
| | | neucleotide | |
| Ectonucleotide | ENPP4 | Metabolism, nucleotide | 6p21.1 |
| pyrophosphatase/phosphodiesterase 4 | | | |
| Cisplatin resistance associated | MTMR11 | Metabolism, | 1q12-q21 |
| | | phospholipid | |
| aAcyl-Coenzyme A oxidase 1, palmitoyl | ACOX1 | Metabolism, | 17q24-q25 |
| | | prostaglandin | |
| Neural precursor cell expressed, developmentally | NEDD4L | Metabolism, ubiquitin- | 18q21 |
| down-regulated 4-like | | protein/ | |
| | | sodium transport | |
| Tetraspanin 7 (transmembrane 4 superfamily, 2) | TSPAN7 | N-linked glycosylation | Xp11.4 |
| Protein tyrosine phosphatase, receptor type, R | PTPRR | Protein | 12q15 |
| | | dephosphorylation | |
| Vacuolar protein sorting 13A (yeast) | VPS13A | Protein localization | 9q21 |
| Procollagen-lysine, 2-oxoglutarate 5-dioxygenase 2 | PLOD2 | Protein modification | 3q23-q24 |
| Dual-specificity tyrosine-(Y)-phosphorylation | DYRK2 | Protein phosphorylation | 12q15 |
| regulated kinase 2 | | | |
| Guanylate cyclase activator 2A (guanylin) | GUCA2A | Intestinal chloride | 1p35-p34 |
| | | secretion | |
| Guanylate cyclase activator 2B (uroguanylin) | GUCA2B | Intestinal chloride | 1p34-p33 |
| | | secretion | |
| Sorcin | SRI | Electrolyte transport | 7q21.1 |
| | | (muscle) | |
| Endothelin 3 | EDN3 | vasoconstriction | 20q13.2-q13.3 |
| Peroxiredoxin 6 | PRDX6 | Response to oxidative | 1q25.1 |
| | | stress | |
| Selenium binding protein 1 | SELENBP1 | Selenium binding | 1q21-q22 |
| A kinase (PRKA) anchor protein (yotiao) 9 | AKAP9 | Signal transduction | 7q21-q22 |
| Phosphoinositide-3-kinase, regulatory subunit, | PIK3R1 | Signal transduction | 5q13.1 |
| polypeptide 1 (p85 alpha) For Peer Review | | | |
| Coagulation factor II (thrombin) receptor-like 1 | F2RL1 | Vascular signal | 5q13 |
| | | transduction | |
| Lectin, galactoside-binding, soluble, 2 (galectin | LGALS2 | Intestinal T cell | 22q13.1 |
| 2) | | regulation | |
| Chromodomain helicase DNA binding protein 1 | CHD1 | Transcription regulation | 5q15-q21 |
| Hepatocyte nuclear factor 4, gamma | HNF4G | Transcription regulation | 8q21.11 |
| Myeloid/lymphoid or mixed-lineage leukemia | MLLT2 | Transcription regulation | 4q21 |
| (trithorax homolog, Drosophila); translocated to, 2 | | | |
| v-myb myeloblastosis viral oncogene homolog | MYB | Transcription regulation | 6q22-q23 |
| (avian) | | | |
| Nuclear receptor subfamily 3, group C, member 2 | NR3C2 | Transcription regulation | 4q31.1 |
| SATB family member 2 | SATB2 | Transcription regulation | 2q33 |
| Zinc finger protein 217 | ZNF217 | Transcription regulation | 20q13.2 |
| Cyclin T2 | CCNT2 | Transcription regulation | 2q21.3 |
| Kruppel-like factor 5 (intestinal) | KLF5 | Transcription regulation | 13q22.1 |
| ATPase, Ca++ transporting, plasma membrane 1 | ATP2B1 | Transport, calcium | 12q21.3 |
| Exophilin 5 | EXPH5 | Transport, protein | 11q22.3 |
| Solute carrier family 16, member 1 | SLC16A1 | Transport, organic | 1p12 |
| | | anion | |
| Secretory carrier membrane protein 1 | SCAMP1 | Transport, protein | 5q13.3-q14.1 |
| Transportin 1 | TNPO1 | Transport, protein | 5q13.2 |
| Solute carrier family 26, member 2 | SLC26A2 | Transport, sulfate | 5q31-q34 |
| Aquaporin 8 | AQP8 | Transport, water | 16p12 |
| Peptidyl arginine deiminase, type II | — | Unknown | 1p35.2-p35.1 |
| Cordon-bleu homolog (mouse) | COBL | Unknown | 7p12.1 |
| Family with sequence similarity 8, member A1 | FAM8A1 | Unknown | 6p22-p23 |
| Hypothetical protein FLJ13910 | FLJ13910 | Unknown | 2p11.2 |
| GRP1-binding protein GRSP1(FERM domain | FRMD4B | Unknown | 13p14.1 |
| containing 4B) | | | |
| Histone 1, H4c | HIST1H4C | Unknown | 6p21.13 |
| Hepatocellular carcinoma antigen gene 520 | LOC63928 | Unknown | 16p12.1 |
| Hypothetical protein LOC92482 | LOC92482 | Unknown | 10q24 |
| FLJ11220 (round spermatid basic protein 1) | RSBN1 | Unknown | 1p13.2 |

Validation of Selected Microarray Results

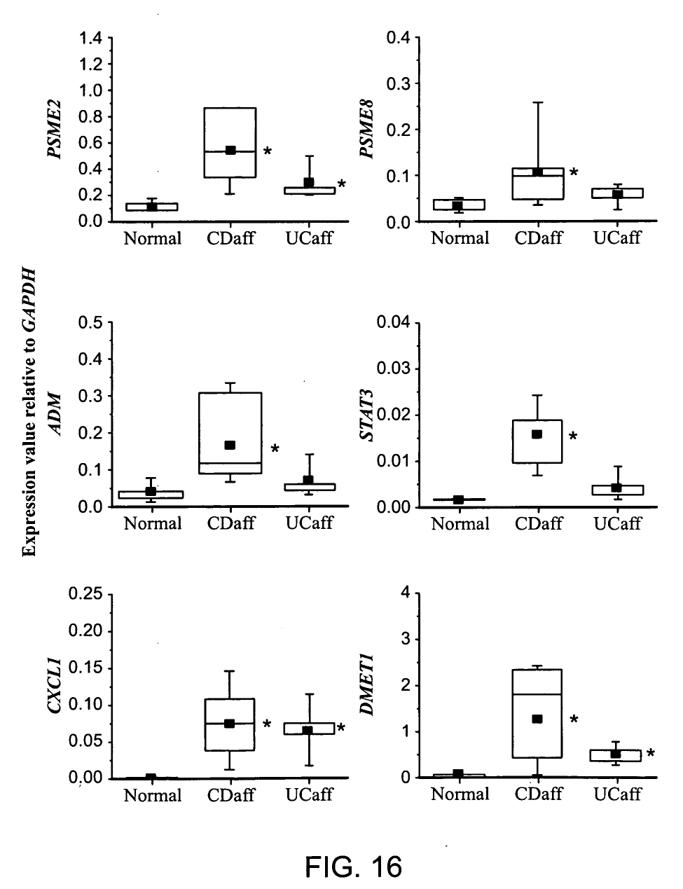

Real-time RT-PCR was used to quantify expression of PSME2, PSMB8, ADM, STAT3, CXCL1 and DMBT in individual biopsy samples to validate selected microarray results. In agreement with the microarray data, the qRT-PCR indicated elevated expression of PSME2, PSMB8, ADM and STAT3 in CD affected biopsies compared to normal controls. The qRT-pCR also indicated elevated PSMB2 mRNA in UC samples while the microarray results had not shown significant over expression of this gene in UC. The CXCL1 and DMBT1 mRNA increased in both CD and UC affected compared to healthy control, confirming the microarray data (FIG. 16).

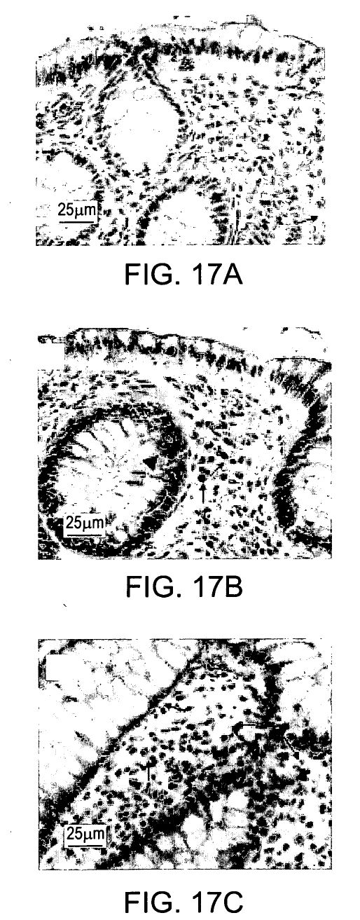

The microarray data indicated that TAP1 (Transporter 1, ATP-binding cassette, sub-family B) was over expressed in CD affected tissues. We further confirmed increase in the TAP1 protein by immunohistochemistry on colon sections (FIG. 17). The results demonstrated more TAP1-positive cells in CD, than in healthy colon biopsy tissue. Furthermore, the TAP1 protein immunostaining was predominantly associated with intestinal macrophages (FIG. 17, arrows), and some crypt epithelial cells in the CD affected biopsy tissues (FIG. 17, arrowhead).

### Genes Differentially Expressed in Unaffected IBD Biopsies Compared to Controls

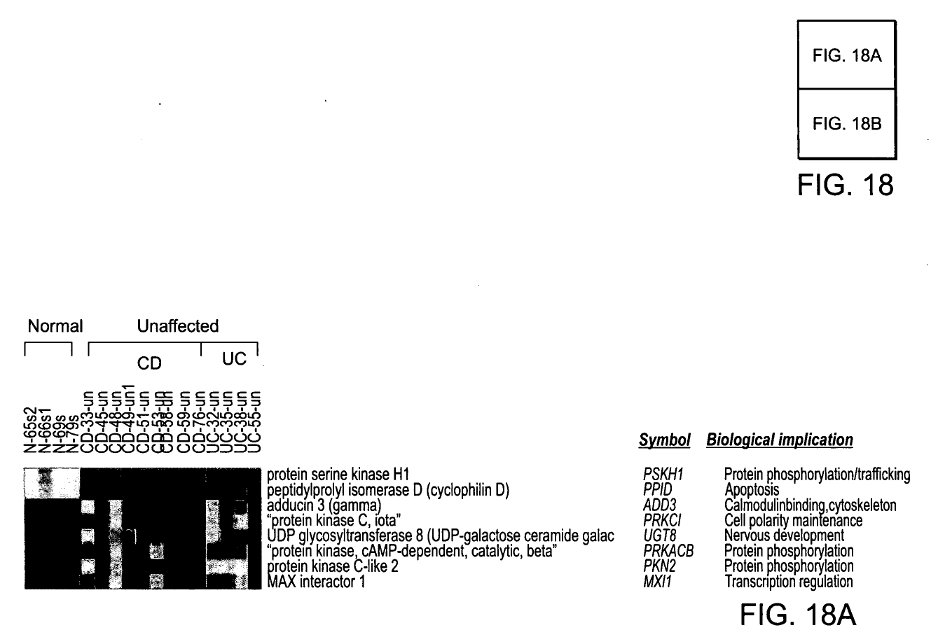

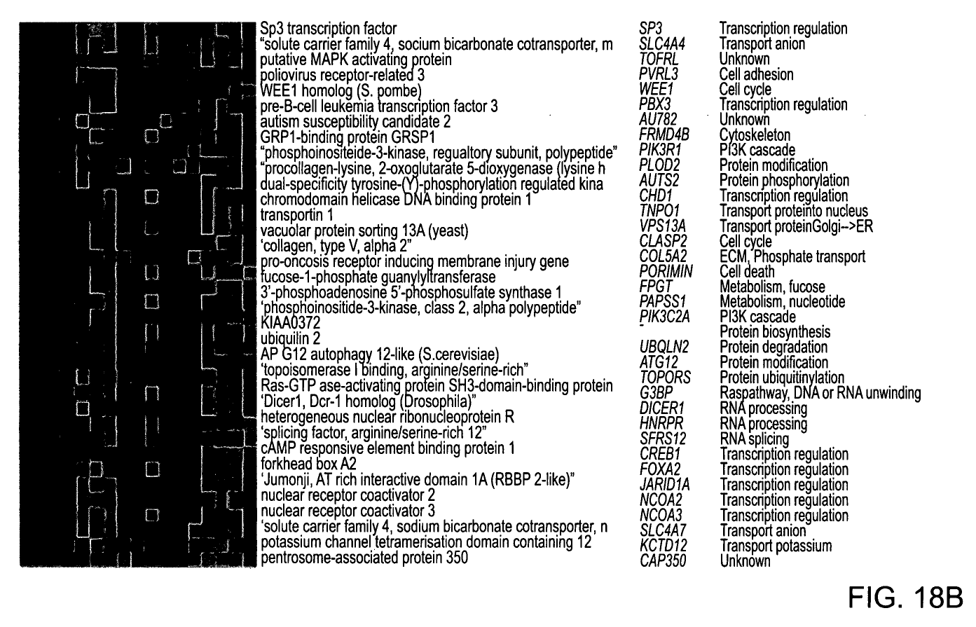

Gene expression patterns of unaffected CD and UC biopsies were compared to normal control samples to identify changes that may reflect systemic processes in patients, or early pathogenic changes that may precede well established disease status as seen in affected areas. SAM analysis revealed approximately 44 differentially expressed genes in the unaffected IBD biopsy samples compared to normal controls. Interestingly, all except two were down regulations compared to normal controls as seen in the heat image of average gene expression (FIG. 18). The down regulations were seen mostly in UC, except CD33, CD45 and CD48 where similar down regulations were observed. The down regulated genes cover a broad range of cell maintenance functions, such as cell polarity, cell adhesion, regulation of transcription, RNA processing, ion transport and protein trafficking. Some of these were also down regulated in UC affected biopsies and noted previously in resected colonic tissue from UC cases. Only two genes, PSKH1 and PPID (peptidyl prolyl isomerase D or cyclophilinD), were significantly over expressed in all unaffected IBD biopsies. These were also over expressed in all IBD affected samples. PSKH1 is a protein serine kinase, involved in the trafficking and processing of pre-mRNA. Cyclophillin D is a mitochondrial matrix pore protein that helps to suppress apoptosis. The biological implications of their over expression in IBD are unclear at this time.

This study elucidated global gene expression patterns of Crohn's disease, ulcerative colitis and two control groups, non-IBD infectious colitis and healthy individuals, using single endoscopic pinch biopsies. Duplicate sampling of the same diseased area of a CD patient indicated the expression patterns to be reproducible and representative of the local diseased area. Unsupervised multidimensional scaling (MDS) of all 36 expression profiles indicated that IBD biopsies were indeed different enough for these to be separated from healthy controls. The fact that the two affected samples from CD76, taken from the same affected area, appear close together in the MDS plot, is a further validation that this method of unsupervised classification is effective, and truly based on gene expression similarities and differences. Infectious colitis was separated from UC and CD affected biopsies, and clearly separated from normal controls and unaffected IBD biopsy samples along component 2. While most CD cases clustered close together in the MDS plot, two CD cases (CD33 and 53) were grouped with UC samples, underscoring the heterogeneous nature of CD. These two CD cases resembled UC, were ANCA-positive²¹ (data not shown) and defined as having high inflammation by histology. The unsupervised multidimensional scaling strategy allows unbiased clustering of samples based on gene expression, and shows the promise of distinguishing active Crohn's and ulcerative colitis tissues from infectious colitis, inactive disease and healthy controls. This approach used on sufficient numbers of cases can ultimately lead to well-defined subgroups and distinguishing subsets of genes and biomarkers.

Following a supervised clustering approach, SAM analysis of each predefined group, UC, CD and infectious colitis (INF) provided some insights into gene expression similarities and dissimilarities between these disease types. Comparing differentially expressed genes in Crohn's and ulcerative colitis, 25/47 or 53% of genes up regulated in CD were also elevated in UC, while 25/51 or 49% of UC-over expressed genes were over expressed in CD. Only 11% (5/47) and 8% (4/51) of these were shared by INF. Among genes down regulated in CD, 18/30 or 60% were also down regulated in UC, while 18/81 or 22% of UC down regulated genes were shared by the CD down regulated profile. In general, many more genes were down regulated in UC than CD, also noted in our previous study of resected tissues⁵. Furthermore, 25% of the genes down regulated in UC were also down regulated in INF and may be reflective of changes underlying common pathogenic mechanisms in inflammation and diarrhea.

The gene expression differences observed between CD and UC speak of distinct biological processes contributing to their pathogenesis. In CD the preferential over expression of interferon γ inducible genes, IFITM1 and IFITM3, as well as STAT1 and STAT3 is indicative of an active TH1 pathway mediated by IL12, IL23 and IFNγ. A recent animal model study indicated a role for IL23 in local intestinal inflammation and colitis,²² while a genome-wide association study identified a significant association between CD and IL23 variants²³. The gene expression profiles can further help to identify specific IL23 responsive regulators of intestinal inflammation in CD. Other over expressed genes in CD consist of the MHC class I antigen processing pathway, such as the immunoproteasome subunit genes (PSME2, PSMB8, PSMB9, PSMB10) that degrade cellular proteins and antigens, and TAP1 encoding the MHC class I transporter associated with antigen processing.²⁴ The UC gene expression pattern is dominated by loss of expression of many genes that regulate metabolism, biosynthesis and electrolyte transport. We speculate that functional loss of specific transcription factors (CCNT2, CHD1, HNF4G, KLF5, MLLT2, MYB, NR3C2, SATB and ZNF217) may play a role in these down regulations. Nuclear receptor superfamily members such as pregnane X receptor (PXR) and the constitutive androstane receptor (CAR) are known to regulate genes required for xenobiotic metabolism and detoxification.^{25, 26} In a study of biopsies taken from surgically removed samples it was proposed that down regulation of PXR in epithelial cells may be responsible for down regulation of several electrolyte transport related genes in UC.⁹ ABCB1/MDR1, in particular has generated some interest in IBD; it was reported as preferentially decreased in UC,⁹ while a genetic study using the gene-wide haplotype tagging approach suggested contribution of ABCB1 variants in UC susceptibility.²⁷ However, in our previous study and the current gene expression pattern of endoscopic biopsy samples, ABCB1 was found to be down regulated in both UC and CD.

One major difference between our earlier study on surgically resected specimen⁵ and the current one is that, in the earlier study we had identified far fewer over expressions for CD. We speculate that surgery in Crohn's disease may occur at a relatively late stage of disease when many genes may be quiescent. In fact several genes detected as over expressed in UC and not CD in that study, were found to be over expressed in CD as well in the current biopsy study, such as, HLA-DRB1, HLA-DMA, LCN2. However, in general many differences noted between CD and UC in our earlier study remained uncontested by the current biopsy study. For example, several immunoglobulin gene transcripts were detected in UC specifically in both studies. When we compared our biopsy gene expression patterns with those of another recent study of endoscopic mucosal biopsy,¹⁴ there were few matches in actual genes identified (BF, NOS2A, TIMP1, upregulated and SLC26A2 down regulated in UC). That study also showed fewer overlaps with other previous IBD gene expression studies. This could be due to the fact that their study used in-house generated cDNA microarrays while many of the studies discussed including ours used high density oligonucleotide microarrays.

A strong motivation for all gene expression studies of complex, heterogeneous diseases like CD and UC, is to complement family-based genetic studies. Baseline expression levels of many genes show familial aggregation.²⁸ Thus, segregation analysis of gene expression data like ours may lead to master regulators of these expression differences that could form the basis of complex diseases like IBD. Second, conventional genome-wide scans have identified numerous IBD susceptibility regions.²⁹⁻³² The finding of candidate genes within these areas by gene expression profiling can lead to identification of disease-susceptibility genes. Potential candidate genes from the expression study include apoptosis-regulating CASP10 at 2q33-34, and antigen-presenting gene PSME2 at 14q11.2 (locus IBD4) from CD profiles, as well as immune response gene IFI30 (19p13.1, IBD6) and Notch-signaling NOTCH3 (19p13.2-p13.1, IBD6) from the UC profiles. With respect to the IBD3 locus at 6p21, 33 HLA-DMA, HLA-DRB1, TAP1, UBD and PSMB8 at 6p21.3, are particularly intriguing.

### REFERENCES

1. Blumberg R S, Strober W. Prospects for research in inflammatory bowel disease. Jama 2001; 285:643-7.
2. Fiocchi C. Inflammatory bowel disease: etiology and pathogenesis. Gastroenterology 1998; 115:182-205.
3. Podolsky D K. Inflammatory bowel disease. N Engl J Med 2002; 347:417-29.
4. Sartor R. Insights into the pathogenesis of inflammatory bowel diseases provided by new rodent models of spontaneous colitis. Inflammatory Bowel Diseases 1995; 1:64-75.
5. Lawrance I C, Fiocchi C, Chakravarti S. Ulcerative colitis and Crohn's disease: distinctive gene expression profiles and novel susceptibility candidate genes. Hum Mol Genet 2001; 10:445-56.
6. Dieckgraefe B, Stenson W, JR Korzenik, Swanson P, Harrington C. Analysis of mucosal gene expression in inflammatory bowel disease by parallel oligonucleotide arrays. Physiol Genomics 2000; 4:1-11.
7. Uthoff S M, Eichenberger M R, Lewis R K, Fox M P, Hamilton C J, McAuliffe T L, Grimes H L, Galandiuk S. Identification of candidate genes in ulcerative colitis and Crohn's disease using cDNA array technology. Int J Oncol 2001; 19:803-10.
8. Dooley T P, Curto E V, Reddy S P, Davis R L, Lambert G W, Wilborn T W, Elson C O. Regulation of gene expression in inflammatory bowel disease and correlation with IBD drugs: screening by DNA microarrays. Inflamm Bowel Dis 2004; 10:1-14.
9. Langmann T, Moehle C, Mauerer R, Scharl M, Liebisch G, Zahn A, Stremmel W, Schmitz G. Loss of detoxification in inflammatory bowel disease: dysregulation of pregnane X receptor target genes. Gastroenterology 2004; 127:26-40.
10. Bayless T M, Tokayer A Z, Polito J M, 2nd, Quaskey S A, Mellits E D, Harris M L. Crohn's disease: concordance for site and clinical type in affected family members—potential hereditary influences. Gastroenterology 1996; 111:573-9.
11. Podolsky D K. Inflammatory bowel disease (1). N Engl J Med 1991; 325:928-37.
12. Picco M F, Bayless T M. Prognostic consideration in idiopathic inflammatory bowel disease. In: Kirsner J B, ed. Inflammatory Bowel Disease. 5th ed. Philadelphia: WB Saunders, 2000:765-780.
13. Hommes D W, van Deventer S J. Endoscopy in inflammatory bowel diseases. Gastroenterology 2004; 126:1561-73.
14. Costello C M, Mah N, Hasler R, Rosenstiel P, Waetzig G H, Hahn A, Lu T, Gurbuz Y, Nikolaus S, Albrecht M, Hampe J, Lucius R, Kloppel G, Eickhoff H, Lehrach H, Lengauer T, Schreiber S. Dissection of the inflammatory bowel disease transcriptome using genome-wide cDNA microarrays. PLoS Med 2005; 2:e199.
15. Colombel J F, Grandbastien B, Gower-Rousseau C, Plegat S, Evrard J P, Dupas J L, Gendre J P, Modigliani R, Belaiche J, Hostein J, Hugot J P, van Kruiningen H, Cortot A. Clinical characteristics of Crohn's disease in 72 families. Gastroenterology 1996; 111:604-7.
16. Li C, Wong W H. Model-based analysis of oligonucleotide arrays: expression index computation and outlier detection. Proc Natl Acad Sci USA 2001; 98:31-6.
17. Torgerson W S. Theory and Methods of Scaling. Wiley, 1958.
18. Taguchi Y H, Oono Y. Relational patterns of gene expression via non-metric multidimensional scaling analysis. Bioinformatics 2005; 21:730-40.
19. Tusher V G, Tibshirani R, Chu G. Significance analysis of microarrays applied to the ionizing radiation response. Proc Natl Acad Sci USA 2001; 98:5116-21.
20. Bates M D, Erwin C R, Sanford L P, Wiginton D, Bezerra J A, Schatzman L C, Jegga A G, Ley-Ebert C, Williams S S, Steinbrecher K A, Warner B W, Cohen M B, Aronow B J. Novel genes and functional relationships in the adult mouse gastrointestinal tract identified by microarray analysis. Gastroenterology 2002; 122:1467-82.
21. Vasiliauskas E A, Kam L Y, Karp L C, Gaiennie J, Yang H, Targan S R. Marker antibody expression stratifies Crohn's disease into immunologically homogeneous subgroups with distinct clinical characteristics. Gut 2000; 47:487-96.
22. Uhlig H H, McKenzie B S, Hue S, Thompson C, Joyce-Shaikh B, Stepankova R, Robinson N, Buonocore S, Tlaskalova-Hogenova H, Cua D J, Powrie F. Differential activity of IL-12 and IL-23 in mucosal and systemic innate immune pathology. Immunity 2006; 25:309-18.
23. Duerr R H, Taylor K D, Brant S R, Rioux J D, Silverberg M S, Daly M J, Steinhart A H, Abraham C, Regueiro M, Griffiths A, Dassopoulos T, Bitton A, Yang H, Targan S, Datta L W, Kistner E O, Schumm L P, Lee A, Gregersen P K, Barmada M M, Rotter J I, Nicolae D L, Cho J H. A Genome-Wide Association Study Identifies IL23R as an Inflammatory Bowel Disease Gene. Science 2006.
24. Begley G S, Horvath A R, Taylor J C, Higgins C F. Cytoplasmic domains of the transporter associated with antigen processing and P-glycoprotein interact with subunits of the proteasome. Mol Immunol 2005; 42:137-41.
25. Waxman D J. P450 gene induction by structurally diverse xenochemicals: central role of nuclear receptors CAR, PXR, and PPAR. Arch Biochem Biophys 1999; 369:11-23.
26. Maglich J M, Stoltz C M, Goodwin B, Hawkins-Brown D, Moore J T, Kliewer S A. Nuclear pregnane x receptor and constitutive androstane receptor regulate overlapping but distinct sets of genes involved in xenobiotic detoxification. Mol Pharmacol 2002; 62:638-46.
27. Ho G T, Soranzo N, Nimmo E R, Tenesa A, Goldstein D B, Satsangi J. ABCB1/MDR1 gene determines susceptibility and phenotype in ulcerative colitis: discrimination of critical variants using a gene-wide haplotype tagging approach. Hum Mol Genet 2006; 15:797-805.
28. Morley M, Molony C M, Weber T M, Devlin J L, Ewens K G, Spielman R S, Cheung V G. Genetic analysis of genome-wide variation in human gene expression. Nature 2004; 430:743-7.
29. Satsangi J, Parkes M, Louis E, Hashimoto L, Kato N, Welsh K, Terwilliger J, Lathrop G, Bell J, Jewell D. Two stage genome-wide search in inflammatory bowel disease provides evidence for susceptibility loci on chromosome 3, 7 and 12. Nature Genet 1996; 14:199-202.
30. Hugot J P, Thomas G. Genome-wide scanning in inflammatory bowel diseases. Dig Dis 1998; 16:364-9.
31. Cho J, Nicolae D, Gold L, Fields C, LaBuda M, Rohal P, Pickles M, Qin L, Fu Y, Mann J, Kirschner B, Jabs E, Weber J, Hanauer S, Bayless T, Brant S. Identification of novel susceptibility loci for inflammatory bowel disease on chromosomes 1p, 3q, and 4q: evidence for epistasis between 1p and IBD1. Proc Natl Acad Sci USA 1998; 95:7502-7.
32. Brant S R, Shugart Y Y. Inflammatory bowel disease gene hunting by linkage analysis: rationale, methodology, and present status of the field. Inflamm Bowel Dis 2004; 10:300-11.
33. Hampe J, Shaw S, Saiz R, Leysens N, Lantermann A, Mascheretti S, Lynch N, MacPherson A, Bridger S, Deventer S v, Stokkers P, Morin P, Mirza M, Forbes A, Lennard-Jones J, Mathew C, Curran M, Schreiber S. Linkage of inflammatory bowel disease to human chromosome 6p. Am. J. Hum. Genet. 1999; 65:1647-1655.

A number of embodiments of the invention have been described. Nevertheless, it will be understood that various modifications may be made without departing from the spirit and scope of the invention. Accordingly, other embodiments are within the scope of the following claims.

1. A biomarker for inflammatory bowel disease status comprising one or more of Markers 1-97, 99-211, 213-264, 266-401 and combinations thereof.

2. The biomarker for inflammatory bowel disease status of claim 1, wherein Markers 1-31, 76-97, 125-136, 187-211, 213-230, 231-264, 266-306 are Markers of Crohn's disease

3. The biomarker for inflammatory bowel disease status of claim 1, wherein Markers 187-230 and 266-306 are Markers of Crohn's disease and ulcerative colitis.

4. (canceled)

5. (canceled)

6. The biomarker for inflammatory bowel disease status of claim 1, wherein one or more of Markers 49-75, 99-124, 137-230, 266-306, and 307-401 are markers of ulcerative colitis.

7.-10. (canceled)

11. The biomarker for inflammatory bowel disease status of claim 1, wherein Markers 1-31 are Markers of Crohn's Disease.

12. The biomarker for inflammatory bowel disease status of claim 1, wherein Markers 32-48 are markers of IBD.

13. The biomarker for inflammatory bowel disease status of claim 1, comprising Markers 1, 2, 4 and 5.

14. The biomarker for inflammatory bowel disease status of claim 1, comprising Markers 6 and 10.

15. The biomarker for inflammatory bowel disease status of claim 1, comprising Markers 17, 18, and 21.

16. (canceled)

17. (canceled)

18. The biomarker for inflammatory bowel disease status of claim 1, comprising Markers 69, 74 and 75.

19.-24. (canceled)

25. A method of qualifying inflammatory bowel disease status in a subject comprising:
(a) measuring at least one biomarker in a sample from the subject, wherein the biomarker is selected from one or more of the biomarkers of Tables 1-9, and
(b) correlating the measurement with inflammatory bowel disease status.

26. The method of claim 25, wherein the inflammatory bowel disease is Crohn's disease or ulcerative colitis.

27. The method of claim 25, further comprising:
(c) managing subject treatment based on the status.

28. The method of claim 27, wherein managing subject treatment is selected from ordering further diagnostic tests, administering at least one therapeutic agent, surgery, surgery followed or preceded by administering at least one therapeutic agent, biotherapy, and taking no further action.

29. The method of claim 28, wherein the therapeutic agent is selected from one or more of sulfa drugs, corticosteriods (prednisone), 5-aminosalicylates (Asacol, Pentasa, Rowasa, or 5-ASA), immunosuppressives (azathioprine, Imuran, Cyclosporine, 6-MP, Purinethol and Methotrexate), anti-TNF (Remicade), anticholinergics, dicyclomine (Bentyl), belladonna/phenobarbital (Donnatal, Antispas, bBarbidonna, donnapine, hyosophen, Spasmolin), hyoscyamine (Levsin, Anaspaz), chlordiazepoxide/clidinium (Librax), anti-diarrheals, diphenoxylate/atropine (Lomotil), alosetron hydrochloride (Lotronex), tegaserod (Zelnorm, Zelmac), rifaximin (Xifaxin), sulfasalazine (Azulfadine), mesalamine (Asacol, Pentasa, Rowasa), osalazine (Dipentum), (Colazal), corticosteroids (prednisone), balsalazide disodium (Colazal®), cyclosporine, methotrexate, infliximab (Remicade), rifaximin, and budesonide (Entocort EC).

30. The method of claim 27, further comprising:
(d) measuring the at least one biomarker after subject management.

31. The method of claim 25, wherein the inflammatory bowel disease status is selected from one or more of the presence or absence of alternating diarrhea and constipation, abdominal pain, bloating, spasms, nausea, bloody diarrhea, fever, dehydration, eye inflammation, joint pain, skin rashes or lesions, mouth ulcers, chronic diarrhea, weight loss, lack of appetite, nutritional deficiencies, and inflamed colon.

32. The method of claim 31, further comprising assessing the status of the inflammatory bowel disease.

33. The method of claim 32, wherein the inflammatory bowel disease status is assessed by barium enema, upper GI series, stool culture, blood tests (to determine a white blood cell count or if anemia is present), fecal occult blood test, sigmoidoscopy, and colonoscopy.

34. A method for differentiating between a diagnosis of inflammatory bowel disease and inflammatory bowel disease comprising:
(a) detecting in a subject sample an amount of at least one biomarker selected from one or more of the biomarkers of Tables 1-9, and
(b) correlating the amount with a diagnosis of inflammatory bowel disease or inflammatory bowel disease.

35. The method of claim 27, wherein the marker is detected by mass spectrometry, PCR, and microarray analysis.

36-40. (canceled)

41. A kit for aiding the diagnosis of inflammatory bowel disease, comprising:
an adsorbent, wherein the adsorbent retains one or more biomarkers selected from one or more of the markers of Tables 1-9, and written instructions for use of the kit for detection of inflammatory bowel disease.

42. A kit for aiding the diagnosis of the subtypes of inflammatory bowel disease, comprising:
an adsorbent, wherein the adsorbent retains one or more biomarkers selected from each of Markers of Tables 1-9, and written instructions for use of the kit for detection of the IBD or a subtype of inflammatory bowel disease, e.g., UC or CD.

43. The kit of claim 41, wherein the instructions provide for contacting a test sample with the adsorbent and detecting one or more biomarkers retained by the adsorbent.

44.-47. (canceled)

48. A method comprising measuring a plurality of biomarkers in a sample from the subject, wherein the biomarkers are selected from one or more of the markers of Tables 1-9.

49. (canceled)

50. (canceled)

51. The method of claim 48, further comprising communicating a diagnosis to a subject, wherein the diagnosis results from the correlation of the biomarkers of Tables 1-9 with inflammatory bowel disease.

52. A method for identifying a candidate compound for treating inflammatory bowel disease comprising:
a) contacting one or more of the biomarkers of Tables 1-9 with a test compound; and
b) determining whether the test compound interacts with the biomarker,
wherein a compound that interacts with the biomarker is identified as a candidate compound for treating inflammatory bowel disease.

53. A method of treating inflammatory bowel disease comprising administering to a subject suffering from or at risk of developing inflammatory bowel disease a therapeutically effective amount of a compound capable of modulating the expression or activity of one or more of the biomarkers of Tables 1-9.

54.-58. (canceled)

59. A purified biomolecule selected from the biomarkers of Tables 1-9.

60-65. (canceled)
